SEQUENCE LISTING (17 sequences)

SEQ ID NO: 1 - protein, 273 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAA

SEQ ID NO: 2 - DNA, 1870 bp, Synthetic construct
  source 1..1870: mol_type = other DNA
gcagttcagcagctgatcatcctgtccacaaaaaagcttgcagaaaaataacattctctgcaagctgatcctgttaaagcttcacaatcactcttccttgaatgcgattttgcaaaatatcttttaacgcacccggcgtttcttccaatgatacttccctgtccacgatggtcagcagctgatcaggcttgagatcagaagacatgcgctcccaaacagcggctctgacgtccatcggacaatatactgaatcgattccgagcaggcttactccgcgaagaataaaaggatacacggttgccggaacttctcctccgccggttaagccgctcactgcgacagatccgccgtattgaattttgcttaaaagcgaggcaagctgttttccgccgactggatcaaccgctccctgccattgctgcttggacagcgccttaagcgttccgtcatagacatcttccctgctgattacttcgcttgcaccaagctgtttcaaataatcagccgcctcccggtttccggtacttgccaccacatcataaccccgcttgttcagcatcgataccgcaattccgccgacaccgccggttgctcctgtgactagcacgctgcctttttccggagacagaccgttctgttcaagccgatgcactgataacgccgcagtaaatcccgccgttccgtacaccatcgcttcttttaacgaaagattctgtggcaaaggcaccagccagtcaccaggcaccgaagcgtattcacttaatccgccatcacgtgagacaccgagctcatagcttgtcgcgatcacctcatccccctccgcaaaacgcggatcattggaagagacgaccgtacccgcagcatcaatgcctaaaataagcggatactctctgacgatattgcctcctgcttttccggccagaccatctttgtaattaatgccggaataagcaactttaatcaggacaccatccttcggcaaatcctctgttgatatggttttcacatggactgaaacatcatcggcatttttttctgcctgcaaggcttgaaataacgttgacattcggcacactccttttcatttatatcgtaaccgaagaacgttcaaaaaaccaaatcatcaagccgccattttcacttcgccggcacattgagacaataatggacaaatccggtatcctcttcatagccgttttgctcatacaagcttcttgccttccggttgtggtgctcagtctgaagtgttaaacattttgccccgttttgccctgcataatcctttgcggcagaaagcagccggccgccggctccctttgtacgcgcatgaggaacgacaaataagtcatttaatatgtatatccttttcattgacacagaagaaaacgttggatagagctgggtaaagcctatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcgctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 3 - DNA, 87 bp, Bacillus subtilis
  source 1..87: mol_type = unassigned DNA
gtgagaagcaaaaaattgtggatcagcttgttgtttgcgttaacgttaatctttacgatggcgttcagcaacatgtctgcgcaggct

SEQ ID NO: 4 - DNA, 228 bp, Bacillus licheniformis
  source 1..228: mol_type = unassigned DNA
gctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttg

SEQ ID NO: 5 - DNA, 825 bp, Synthetic construct
  source 1..825: mol_type = other DNA
gcgcaaaccgttccttacggcattcctctcattaaagcggacaaagtgcaggctcaaggctttaagggagcgaatgtaaaagtagccgtcctggatacaggaatccaagcatctcatccggacttgaacgtagtcggcggagcgagctttgtggctggcgaagcatataacaccgacggcaacggacacggcacacatgttgccggtacagtagctgcgcttgacaatacaacgggtgtattaggcgttgcgccaagcgtatccttgtacgcggttaaagtactgaactcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatacgcaagaggggttgtcgttgtagcagcagctgggaacagcggatcttcaggaaacacgaatacaattggctatcctgcgaaatacgattctgtcatcgctgttggcgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaataa

SEQ ID NO: 6 - DNA, 296 bp, Synthetic construct
  source 1..296: mol_type = other DNA
tagggcataaggtggtcgcgaatgtgccctgccacgttggacctctagatacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaat

SEQ ID NO: 7 - DNA, 3169 bp, Synthetic construct
  source 1..3169: mol_type = other DNA
agtctttcgactgagcctttcgttttatttgatgcctcaagctagagagtcattaccagatctcactgcagtcactactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatactcatctctccctttttttaaaaatttccccttttaaaattaaaccaaaacccaggagcaaaaacaacacatgatgtatacatcttctttaatccaaaaagaaaatgcttacattaaatcgtttttcaaataaaacaatcgtgcgtttctatataccgttcgatactcgatatgcttaataaagattaagcatatttatattataaacaattatcagacatttgttaaatgtttaactatagttttttttcagccttattctcctgataacgcgagacagcattagaaaaaggcgtaaccgcaaagctcaaaacagaaaacaaaagcaataacagcggaagtgccgcaagatcatgccgcccttctaaatgaaacatgctgcgggttaggcgaaccgtccgcttgtaaagcttatcaatgacataaaatccggcgagcgacacgagcaaatagccagccagaccgatgtaaacgtgcttcatgacataatggcccatttcgtggcccataataaacagaatttctgaatcgtcaagtttgttcagcgtcgtatcccacaatacaatccgtttattggccccaattcctgtaacataggcattcagcgcatttgttttttctgacatgttcacttcatatacatggtcagccggaatattggcttcatctgccagctctaaaattttgctttcaagctctttgtttttcagcggataaaaatcattgtataaaggatcgataatgaccggctgaataaaaaacagaaacagcgaaaacggcactgttaacagccaggcgtataaccaccattttttttcatgccttttgatcagccaataaaaaacgagaacgcaaagcgtaaagattggaaagctgatccaaaagctgataacctgatccttagcccagctggccgttgtctgtgtggaaatgttatagtcaagcgatacttgatagcctatccaatctaaaggcagcgtcaccaatgttgtaatcagcgaaagcacaaacacaaaaccaacggtctgcaaaaaccgaaaaggcacggccgcttcgatccatttcttgattttctttgaaacaccgctgacaagcagaacaaaaaacagaaaccaatcaagtggtaccccgataaaaaataaaaaattcttgacattcgaatactgctcggccactgccaactcagacggcttcatgaaagaagccggatcagcctgcgtccctttcacggcttccggtattgcggaatcacccgacaggaaaaaataccaataaaaaaacagcccgtacagcacgtaagcaagtcctgctgccgcaatccacttgcgcataacgcctcactcctcacatcaacccgttacttctattgtaatcataaattcaaattcttagaaccaagctgtgttccgcacttttccacccttttaagcatggaaaccccgatcgctgggaaaactaacaatgtttggagtgatgcaaatgaaaaaaatagtggcagccatcgtggtaatcggtcttgtgtttatcgcatttttttatctttacagccgatcaggcgatgtgtatcaatcggtagacgcggatttgatcacactgtcttcaagcggccaggaagatatcgagattgaaaaaagacagcacgtcaaagatatgctggatattatgaatcagggaaaacaggtgaagacagaaaaaacatcagcccctgattacgaagggacaatcaagtttcataaagaccggtatgactcattcagactatggattgacggcagccagcaagccgtttttttgaaggatggcacatactacaaattaagcaaaaatgatacaaaggcgctgctaaatattattaaaaaagaagcaaaggattgaaaatgaaaaagcgaagctaaccgcttcgctttttcattttattggggcaaaatatctctcagtgcccgtctgagcattttccccgtcgcatttttcggaatatcgtcaagaaacgtaatggcggcaggccgcttgtattttgccagatgcttttcgcagtgctgcatgatgtcctcctctgttaccccagagcgtttcggcaccacatatccctttaccgcttccccgctttgggggtccggcacgccgatgacaaccgcctccttgacgtccggatggctgtacagcacctcctccacctcccgcggatacacattgtatcctcctacaatgatcatgtcttttttccggtcaacaatgtaaaaatagccgtcctcatcccgtcttgccaagtcccccgtataaagccacccgtctttta

SEQ ID NO: 8 - protein, 274 aa, Synthetic construct
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSSGSGSYSGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 9 - DNA, 4664 bp, Synthetic construct
  source 1..4664: mol_type = other DNA
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaagtcgctgataaacagctgacatcaatatcctattttttcaaaaaatattttaaaaagttgttgacttaaaagaagctaaatgttatagtaataaa

SEQ ID NO: 10 - DNA, 58 bp, Bacillus subtilis
  source 1..58: mol_type = unassigned DNA
acagaatagtcttttaagtaagtctactctgaatttttttaaaaggagagggtaaaga

SEQ ID NO: 11 - DNA, 87 bp, Bacillus licheniformis
  source 1..87: mol_type = unassigned DNA
atgatgaggaaaaagagtttttggcttgggatgctgacggccttcatgctcgtgttcacgatggcattcagcgattccgcttctgct

SEQ ID NO: 12 - DNA, 40 bp, Bacillus licheniformis
  source 1..40: mol_type = unassigned DNA
agaggacggatttcctgaaggaaatccgtttttttatttt

SEQ ID NO: 13 - DNA, 3197 bp, Bacillus licheniformis
  source 1..3197: mol_type = unassigned DNA
acagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtcaacttttttgtcttcaccttcaatttctacctgcttaatatctttagcagcttctttataagaactttgaatatcgatcatcttgtccagtgttaagtttgttttgatgtttttttctaatgctgccaaaatctcttgatagtttgttaatgattttaattgtgcaccttcatgaataatttcgttgataatctcacgctgacgctcttgac

SEQ ID NO: 14 - DNA, 4541 bp, Synthetic construct
  source 1..4541: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcaagctgataaacagctgacatcaactaaaagtttcattaaatactttgaaaaaagttgttgacttaaaagaagctaaatgttatagtaattgt

SEQ ID NO: 15 - DNA, 3019 bp, Bacillus licheniformis
  source 1..3019: mol_type = unassigned DNA
caagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacg

SEQ ID NO: 16 - DNA, 95 bp, Synthetic construct
  source 1..95: mol_type = other DNA
gtcgctgataaacagctgacatcaatatcctattttttcaaaaaatattttaaaaagttgttgacttaaaagaagctaaatgttatagtaataaa

SEQ ID NO: 17 - DNA, 91 bp, Synthetic construct
  source 1..91: mol_type = other DNA
gctgataaacagctgacatcaactaaaagtttcattaaatactttgaaaaaagttgttgacttaaaagaagctaaatgttatagtaattgt

(19) United States
(12) Patent Application Publication
(10) Pub. No.: US 2026/0078362 A1
(43) Pub. Date: Mar. 19, 2026

(54) SUBTILISIN VARIANTS AND METHODS RELATED THERETO

(71) Applicant: Danisco US Inc., Palo Alto, CA (US)

(72) Inventors: Joshua Bassler, Palo Alto, CA (US); Zachary Q. Beck, Palo Alto, CA (US); Thomas P. Graycar, Pacifica, CA (US); Michael Stoner, San Jose, CA (US)

(21) Appl. No.: 19/107,312

(22) PCT Filed: Aug. 29, 2023
(86) PCT No.: PCT/US2023/073059

§ 371 Date: Feb. 27, 2025

Related U.S. Application Data
(60) Provisional application No. 63/403,330, filed on Sep. 2, 2022.
(60) Provisional application No. 63/492,614, filed on Mar. 28, 2023.

(51) Int. Cl. C12N 9/54; C11D 3/386

(52) U.S. Cl. CPC C12N 9/54; C11D 3/38618; C12Y 304/21062

(57) ABSTRACT

Disclosed herein is one or more subtilisin variant, nucleic acid encoding same, and compositions and methods related to the production and use thereof, including one or more subtilisin variant that has improved stability and/or soil removal compared to one or more reference subtilisin.

### CROSS-REFERENCE TO RELATED APPLICATIONS

The present application is a 371 of International Application No. PCT/US2023/073059, filed Aug. 29, 2023 and claims the benefit of U.S. Provisional Patent Application No. 63/403,330, filed Sep. 2, 2022, and U.S. Provisional Patent Application No. 63/492,614 filed Mar. 28, 2023, which are incorporated in their entirety by reference.

Disclosed herein is one or more subtilisin variant, nucleic acid encoding same, and compositions and methods related to the production and use thereof, including one or more subtilisin variant that has improved stability and/or soil removal compared to one or more reference subtilisin.

### REFERENCE TO SEQUENCE LISTING SUBMITTED ELECTRONICALLY

The content of the sequence listing electronically submitted with the application as an XML file (Name: NB42132PCT_SequenceListing.xml; Size: 38,509 bytes; Created: Aug. 25, 2023) forms part of the application and is hereby incorporated herein by reference in its entirety.

### BACKGROUND

A protease (also known as a proteinase) is an enzyme that has the ability to break down other proteins. A protease has the ability to conduct proteolysis, which begins protein catabolism by hydrolysis of peptide bonds that link amino acids together in a peptide or polypeptide chain forming the protein. This activity of a protease as a protein-digesting enzyme is termed a proteolytic activity. Many well-known procedures exist for measuring proteolytic activity (Kalisz, “Microbial Proteinases,” In: Fiechter (ed.), Advances in Biochemical Engineering/Biotechnology, (1988)). For example, proteolytic activity may be ascertained by comparative assays which analyze the respective protease's ability to hydrolyze a commercial substrate. Exemplary substrates useful in the analysis of protease or proteolytic activity, include, but are not limited to, di-methyl casein (Sigma C-9801), bovine collagen (Sigma C-9879), bovine elastin (Sigma E-1625), and Keratin Azure (Sigma-Aldrich K8500). Colorimetric assays utilizing these substrates are well known in the art (see, e.g., WO 99/34011 and U.S. Pat. No. 6,376,450, both of which are incorporated herein by reference).

Serine proteases are enzymes (EC No. 3.4.21) possessing an active site serine that initiates hydrolysis of peptide bonds of proteins. Serine proteases comprise a diverse class of enzymes having a wide range of specificities and biological functions that are further divided based on their structure into chymotrypsin-like (trypsin-like) and subtilisin-like. The prototypical subtilisin (EC No. 3.4.21.62) was initially obtained from Bacillus subtilis. Subtilisins and their homologues are members of the S8 peptidase family of the MEROPS classification scheme (Rawlings, N. D. et al (2016) Twenty years of the MEROPS database of proteolytic enzymes, their substrates and inhibitors. Nucleic Acids Res 44, D343-D350). Members of family S8 have a catalytic triad in the order Asp, His and Ser in their amino acid sequence. Although a number of useful variant proteases have been developed for cleaning applications, there remains a need for improved protease variants.

### SUMMARY

One embodiment is directed to a subtilisin variant comprising one, two or more substitutions selected from the group consisting of X9T, X17H, X45R, X68S, X78I, X86E, X87A, X96D, X100E, X100N, X103F, X103I, X108Q, X115L, X117R, X127S, X127T, X128K, X128P, X128R, X129Q, X155E, X161Q, X181E, X181Q, X202V, X203E, X203N, X217S, X221Q, X260W, and X264H where the positions are numbered according to SEQ ID NO: 1, and where the variant has at least 75% identity to the amino acid sequence of SEQ ID NO: 1 or SEQ ID NO: 8. In some embodiments, the subtilisin variants have at least 25% improved stability in detergent as compared to the parent subtilisin SEQ ID NO: 1 and/or a net charge of −4 to +2 at pH 8 relative to the subtilisin having the amino acid sequence of SEQ ID NO: 1.

In another embodiment, the disclosure is directed to variant subtilisin, where the variant comprises an amino acid sequence having at least 75% sequence identity to the amino acid sequence of SEQ ID NO: 1 and has at least one, two or more substitutions selected from the group consisting of X9T, X17H, X45R, X68S, X78I, X86E, X87A, X96D, X100E, X100N, X103F, X103I, X108Q, X115L, X117R, X127S, X127T, X128K, X128P, X128R, X129Q, X155E, X161Q, X181E, X181Q, X202V, X203E, X203N, X217S, X221Q, X260W, and X264H where the positions are numbered according to the amino acid sequence of SEQ ID NO: 1, and where the variant has a net charge of −4 to +2 at pH 8 relative to the subtilisin having the amino acid sequence of SEQ ID NO: 1. In some embodiments, the subtilisin variants have at least 25% improved stability in detergent as compared to the parent subtilisin SEQ ID NO: 1.

Still other embodiments are directed to a method for producing a variant described herein, comprising stably transforming a host cell with an expression vector comprising a polynucleotide encoding one or more subtilisin variant described herein. Still further embodiments are directed to a polynucleotide comprising a nucleic acid sequence encoding one or more subtilisin variant described herein.

### DESCRIPTION

In one embodiment, the present disclosure provides one or more subtilisin variant comprising one, two, three or more amino acid substitutions at a position selected from the group consisting of 9, 17, 45, 68, 78, 86, 87, 96, 100, 103, 108, 115, 117, 127, 128, 129, 155, 161, 181, 202, 203, 217, 221,260, and 264 where the positions are numbered by correspondence with the amino acid sequence of SEQ ID NO: 1. In some embodiments, the variants provided herein demonstrate one or more improved properties, such as an improved stability or improved cleaning performance, or both an improved stability and an improved cleaning performance when compared to a subtilisin having the amino acid sequence of SEQ ID NO: 1. In some embodiments, the subtilisin variant comprises at least two, three or more substitutions selected from the group consisting of X9T, X17H, X45R, X68S, X78I, X86E, X87A, X96D, X100E, X100N, X103F, X103I, X108Q, X115L, X117R, X127S, X127T, X128K, X128P, X128R, X129Q, X155E, X161Q, X181E, X181Q, X202V, X203E, X203N, X217S, X260W, X221Q, and X264H, and where the positions are numbered according to SEQ ID NO: 1, and where the variant has at least 75% identity to the amino acid sequence of SEQ ID NO: 1 or SEQ ID NO: 8. In further embodiments, the subtilisin variant further comprises one or more additional mutations selected from the group consisting of X24Q, X77N, X86D, X165Q, X184Q, X258D, and X258P, wherein the positions are numbered according to SEQ ID NO: 1. The subtilisin variants provided herein find use in the preparation of cleaning compositions (e.g. automatic dishwashing compositions or laundry detergent compositions). In addition, the subtilisin variants provided herein also find use in methods of cleaning (e.g. dish washing methods or laundry washing methods) using such variants or compositions comprising such subtilisin variants.

Unless otherwise indicated herein, one or more subtilisin variant described herein can be made and used by a variety of techniques used in molecular biology, microbiology, protein purification, protein engineering, protein and DNA sequencing, recombinant DNA fields, and industrial enzyme use and development. Terms and abbreviations not defined should be accorded their ordinary meaning as used in the art. Unless defined otherwise herein, all technical and scientific terms used herein have the same meaning as commonly understood by one of ordinary skill in the art. Any definitions provided herein are to be interpreted in the context of the specification as a whole. As used herein, the singular “a,” “an” and “the” includes the plural unless the context clearly indicates otherwise. Unless otherwise indicated, nucleic acid sequences are written left to right in 5′ to 3′ orientation; and amino acid sequences are written left to right in amino to carboxy orientation. Each numerical range used herein includes every narrower numerical range that falls within such broader numerical range, as if such narrower numerical ranges were all expressly written herein.

As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

The nomenclature of the amino acid substitutions of the one or more subtilisin variants described herein uses one or more of the following: position; position: amino acid substitution(s); or starting amino acid(s): position: amino acid substitution(s). Reference to a “position” (e.g. 5, 8, 17, 22, etc) encompasses any starting amino acid that may be present at such position, and any substitution that may be present at such position. Reference to a “position: amino acid substitution(s)” (e.g. 1S/T/G, 3G, 17T, etc) encompasses any starting amino acid that may be present at such position and the one or more amino acid(s) with which such starting amino acid may be substituted. Reference to a position can be recited in several forms, for example, position 003 can also be referred to as position 03 or 3. Reference to a starting or substituted amino acid may be further expressed as several starting, or substituted amino acids separated by a foreslash (“/”). For example, D275S/K indicates position 275 is substituted with serine(S) or lysine (K) and P/S197K indicates that starting amino acid proline (P) or serine(S) at position 197 is substituted with lysine (K). Reference to an X as the amino acid in a position, refers to any amino acid at the recited position.

The position of an amino acid residue in a given amino acid sequence is numbered by correspondence with the amino acid sequence of SEQ ID NO:1. That is, the amino acid sequence of SEQ ID NO: 1 serves as a reference sequence for numbering of positions of an amino acid residue. For example, the amino acid sequence of one or more subtilisin variant described herein is aligned with the amino acid sequence of SEQ ID NO: 1 using an alignment algorithm as described herein, and each amino acid residue in the given amino acid sequence that aligns (preferably optimally aligns) with an amino acid residue in SEQ ID NO: 1 is conveniently numbered by reference to the numerical position of that corresponding amino acid residue. Sequence alignment algorithms, such as, for example, described herein will identify the location or locations where insertions or deletions occur in a subject sequence when compared to a query sequence (also sometimes referred to as a “reference sequence”). Sequence alignment with other subtilisin amino acid sequences can be determined using an amino acid alignment, for example, as provided in FIG. 1 of PCT Publication No. WO2018118917.

The terms “protease” and “proteinase” refer to an enzyme that has the ability to break down proteins and peptides. A protease has the ability to conduct “proteolysis,” by hydrolysis of peptide bonds that link amino acids together in a peptide or polypeptide chain forming the protein. This activity of a protease as a protein-digesting enzyme is referred to as “proteolytic activity.” Many well-known procedures exist for measuring proteolytic activity. For example, proteolytic activity may be ascertained by comparative assays that analyze the respective protease's ability to hydrolyze a suitable substrate. Exemplary substrates useful in the analysis of protease or proteolytic activity, include, but are not limited to, di-methyl casein (Sigma C-9801), bovine collagen (Sigma C-9879), bovine elastin (Sigma E-1625), and Keratin Azure (Sigma-Aldrich K8500). Colorimetric assays utilizing these substrates are well known in the art (See e.g., WO99/34011 and U.S. Pat. No. 6,376,450). The pNA peptidyl assay (Sec e.g., Del Mar et al., Anal Biochem, 99:316-320, 1979) also finds use in determining the active enzyme concentration. This assay measures the rate at which p-nitroaniline is released as the enzyme hydrolyzes a soluble synthetic substrate, such as succinyl-alanine-alanine-proline-phenylalanine-p-nitroanilide (suc-AAPF-pNA). The rate of production of yellow color from the hydrolysis reaction is measured at 405 or 410 nm on a spectrophotometer and is proportional to the active enzyme concentration. In addition, absorbance measurements at 280 nanometers (nm) can be used to determine the total protein concentration in a sample of purified protein. The activity on substrate divided by protein concentration gives the enzyme specific activity.

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. gibsonii, B. pumilus, Bacillus xiamenensis, B sp TY-145 and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”, or B. polymyxa, which is now “Paenibacillus polymyxa”. The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

The term “vector” refers to a nucleic acid construct used to introduce or transfer nucleic acid(s) into a target cell or tissue. A vector is typically used to introduce foreign DNA into a cell or tissue. Vectors include plasmids, cloning vectors, bacteriophages, viruses (e.g., viral vector), cosmids, expression vectors, shuttle vectors, and the like. A vector typically includes an origin of replication, a multicloning site, and a selectable marker. The process of inserting a vector into a target cell is typically referred to as transformation. The present invention includes, in some embodiments, a vector that comprises a DNA sequence encoding a serine protease polypeptide (e.g., precursor or mature serine protease polypeptide) that is operably linked to a suitable prosequence (e.g., secretory, signal peptide sequence, etc.) capable of effecting the expression of the DNA sequence in a suitable host, and the folding and translocation of the recombinant polypeptide chain.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, electroporation, conjugation, and transduction. Transformation refers to the genetic alteration of a cell which results from the uptake, optional genomic incorporation, and expression of genetic material (e.g., DNA).

The term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any step involved in the “production of the polypeptide” including, but not limited to, transcription, post-transcriptional modifications, translation, post-translational modifications, secretion and the like.

The phrases “expression cassette” or “expression vector” refers to a nucleic acid construct or vector generated recombinantly or synthetically for the expression of a nucleic acid of interest (e.g., a foreign nucleic acid or transgene) in a target cell. The nucleic acid of interest typically expresses a protein of interest. An expression vector or expression cassette typically comprises a promoter nucleotide sequence that drives or promotes expression of the foreign nucleic acid. The expression vector or cassette also typically includes other specified nucleic acid elements that permit transcription of a particular nucleic acid in a target cell. A recombinant expression cassette can be incorporated into a plasmid, chromosome, mitochondrial DNA, plastid DNA, virus, or nucleic acid fragment. Some expression vectors have the ability to incorporate and express heterologous DNA fragments in a host cell or genome of the host cell. Many prokaryotic and eukaryotic expression vectors are commercially available. Selection of appropriate expression vectors for expression of a protein from a nucleic acid sequence incorporated into the expression vector is within the knowledge of those of skill in the art.

As used herein, a nucleic acid is “operably linked” with another nucleic acid sequence when it is placed into a functional relationship with another nucleic acid sequence. For example, a promoter or enhancer is operably linked to a nucleotide coding sequence if the promoter affects the transcription of the coding sequence. A ribosome binding site may be operably linked to a coding sequence if it is positioned so as to facilitate translation of the coding sequence. Typically, “operably linked” DNA sequences are contiguous. However, enhancers do not have to be contiguous. Linking is accomplished by ligation at convenient restriction sites. If such sites do not exist, synthetic oligonucleotide adaptors or linkers may be used in accordance with conventional practice.

The term “gene” refers to a polynucleotide (e.g., a DNA segment), that encodes a polypeptide and includes regions preceding and following the coding regions. In some instances, a gene includes intervening sequences (introns) between individual coding segments (exons).

The term “recombinant”, when used with reference to a cell typically indicates that the cell has been modified by the introduction of a foreign nucleic acid sequence or that the cell is derived from a cell so modified. For example, a recombinant cell may comprise a gene not found in identical form within the native (non-recombinant) form of the cell, or a recombinant cell may comprise a native gene (found in the native form of the cell) that has been modified and re-introduced into the cell. A recombinant cell may comprise a nucleic acid endogenous to the cell that has been modified without removing the nucleic acid from the cell; such modifications include those obtained by gene replacement, site-specific mutation, and related techniques known to those of ordinary skill in the art. Recombinant DNA technology includes techniques for the production of recombinant DNA in vitro and transfer of the recombinant DNA into cells where it may be expressed or propagated, thereby producing a recombinant polypeptide. “Recombination” and “recombining” of polynucleotides or nucleic acids refer generally to the assembly or combining of two or more nucleic acid or polynucleotide strands or fragments to generate a new polynucleotide or nucleic acid.

A nucleic acid or polynucleotide is said to “encode” a polypeptide if, in its native state or when manipulated by methods known to those of skill in the art, it can be transcribed and/or translated to produce the polypeptide or a fragment thereof. The anti-sense strand of such a nucleic acid is also said to encode the sequence.

The terms “host strain” and “host cell” refer to a suitable host for an expression vector comprising a DNA sequence of interest.

A “protein” or “polypeptide” comprises a polymeric sequence of amino acid residues. The terms “protein” and “polypeptide” are used interchangeably herein. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code.

The terms “prosequence” or “propeptide sequence” refer to an amino acid sequence between the signal peptide sequence and mature protease sequence that is necessary for the proper folding and secretion of the protease; they are sometimes referred to as intramolecular chaperones. Cleavage of the prosequence or propeptide sequence results in a mature active protease. Bacterial serine proteases are often expressed as pro-enzymes. Examples of modified propeptides are provided, for example, in WO 2016/205710.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “mature” form of a protein, polypeptide, or peptide refers to the functional form of the protein, polypeptide, or peptide without the signal peptide sequence and propeptide sequence.

The term “precursor” form of a protein or peptide refers to a mature form of the protein having a prosequence operably linked to the amino or carbonyl terminus of the protein. The precursor may also have a “signal” sequence operably linked to the amino terminus of the prosequence. The precursor may also have additional polypeptides that are involved in post-translational activity (e.g., polypeptides cleaved therefrom to leave the mature form of a protein or peptide).

The term “wildtype”, with respect to a polypeptide, refers to a naturally-occurring polypeptide that does not include a man-made substitution, insertion, or deletion at one or more amino acid positions. Similarly, the term “wildtype”, with respect to a polynucleotide, refers to a naturally-occurring polynucleotide that does not include a man-made substitution, insertion, or deletion at one or more nucleotides. A polynucleotide encoding a wildtype polypeptide is, however, not limited to a naturally-occurring polynucleotide, and encompasses any polynucleotide encoding the wildtype or parental polypeptide.

The term “parent”, with respect to a polypeptide, includes reference to a naturally-occurring, or wildtype, polypeptide or to a naturally-occurring polypeptide in which a man-made substitution, insertion, or deletion at one or more amino acid positions has been made that serves as the basis for introducing substitutions or additional substitutions to produce the variant enzymes provided herein. The term “parent” with respect to a polypeptide also includes any polypeptide that has protease activity that serves as the starting polypeptide for alteration, such as substitutions, additions, and/or deletions, to result in a variant having one or more alterations in comparison to the starting polypeptide. That is, a parental, or reference polypeptide is not limited to a naturally-occurring wildtype polypeptide, and encompasses any wildtype, parental, or reference polypeptide. Similarly, the term “parent,” with respect to a polynucleotide, can refer to a naturally-occurring polynucleotide or to a polynucleotide that does include a man-made substitution, insertion, or deletion at one or more nucleotides. The term “parent” with respect to a polynucleotide also includes any polynucleotide that encodes a polypeptide having protease activity that serves as the starting polynucleotide for alteration to result in a variant protease having a modification, such as substitutions, additions, and/or deletions, in comparison to the starting polynucleotide. That is, a polynucleotide encoding a wildtype, parental, or reference polypeptide is not limited to a naturally-occurring polynucleotide, and encompasses any polynucleotide encoding the wildtype, parental, or reference polypeptide. In some embodiments, the parent polypeptide herein, comprises a polypeptide having the amino acid sequence set forth in SEQ ID NO:1.

The term “naturally-occurring” refers to, for example, a sequence and residues contained therein (e.g., polypeptide sequence and amino acids contained therein or nucleotide sequence and nucleotides contained therein) that are found in nature. Conversely, the term “non-naturally occurring” refers to, for example, a sequence and residues contained therein (e.g., polypeptide sequences and amino acids contained therein or nucleotide sequence and nucleic acids contained therein) that are not found in nature.

As used herein with regard to amino acid residue positions, “corresponding to” or “corresponds to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question. To exemplify, “proteases derived from Bacillus” refers to those enzymes having proteolytic activity that are naturally produced by Bacillus, as well as to serine proteases like those produced by Bacillus sources but which through the use of genetic engineering techniques are produced by other host cells transformed with a nucleic acid encoding the serine proteases.

The term “identical” in the context of two polynucleotide or polypeptide sequences refers to the nucleotides or amino acids in the two sequences that are the same when aligned for maximum correspondence, as measured using sequence comparison or analysis algorithms described below and known in the art.

The phrases “% identity” or “percent identity” or “PID” refers to protein sequence identity. Percent identity may be determined using standard techniques known in the art. The percent amino acid identity shared by sequences of interest can be determined by aligning the sequences to directly compare the sequence information, e.g., by using a program such as BLAST, MUSCLE, or CLUSTAL. The BLAST algorithm is described, for example, in Altschul et al., J Mol Biol, 215:403-410 (1990) and Karlin et al., Proc Natl Acad Sci USA, 90:5873-5787 (1993). A percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence.

As used herein, “homologous proteins” or “homologous proteases” refers to proteins that have distinct similarity in primary, secondary, and/or tertiary structure. Protein homology can refer to the similarity in linear amino acid sequence when proteins are aligned. Homology can be determined by amino acid sequence alignment, e.g., using a program such as BLAST, MUSCLE, or CLUSTAL. Homologous search of protein sequences can be done using BLASTP and PSI-BLAST from NCBI BLAST with threshold (E-value cut-off) at 0.001. (Altschul et al., “Gapped BLAST and PSI BLAST a new generation of protein database search programs”, Nucleic Acids Res, Set 1; 25 (17): 3389-402 (1997)). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997 and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. Using this information, protein sequences can be grouped and/or a phylogenetic tree built therefrom. Amino acid sequences can be entered in a program such as the Vector NTI Advance suite and a Guide Tree can be created using the Neighbor Joining (NJ) method (Saitou and Nei, Mol Biol Evol, 4:406-425, 1987). The tree construction can be calculated using Kimura's correction for sequence distance and ignoring positions with gaps. A program such as AlignX can display the calculated distance values in parenthesis following the molecule name displayed on the phylogenetic tree.

Understanding the homology between molecules can reveal the evolutionary history of the molecules as well as information about their function; if a newly sequenced protein is homologous to an already characterized protein, there is a strong indication of the new protein's biochemical function. Two molecules are said to be homologous if they have been derived from a common ancestor. Homologous molecules, or homologs, can be divided into two classes, paralogs and orthologs. Paralogs are homologs that are present within one species. Paralogs often differ in their detailed biochemical functions. Orthologs are homologs that are present within different species and have very similar or identical functions. A protein superfamily is the largest grouping (clade) of proteins for which common ancestry can be inferred. Usually, this common ancestry is based on sequence alignment and mechanistic similarity. Superfamilies typically contain several protein families which show sequence similarity within the family. The term “protein clan” is commonly used for protease superfamilies based on the MEROPS protease classification system. As used herein, the term “subtilisin” includes any member of the S8 serine protease family as described in MEROPS—The Peptidase Data base (Rawlings, N. D., et al (2016) Twenty years of the MEROPS database of proteolytic enzymes, their substrates and inhibitors. Nucleic Acids Res 44, D343-D350).

The CLUSTAL W algorithm is another example of a sequence alignment algorithm (See, Thompson et al., Nucleic Acids Res, 22:4673-4680, 1994). Default parameters for the CLUSTAL W algorithm include: Gap opening penalty=10.0; Gap extension penalty=0.05; Protein weight matrix=BLOSUM series; DNA weight matrix=IUB; Delay divergent sequences %=40; Gap separation distance=8; DNA transitions weight=0.50; List hydrophilic residues=GPSNDQEKR; Use negative matrix=OFF; Toggle Residue specific penalties=ON; Toggle hydrophilic penalties=ON; and Toggle end gap separation penalty=OFF. In CLUSTAL algorithms, deletions occurring at either terminus are included. For example, a variant with a five amino acid deletion at either terminus (or within the polypeptide) of a polypeptide of 500 amino acids would have a percent sequence identity of 99% (495/500 identical residues×100) relative to the “reference” polypeptide. Such a variant would be encompassed by a variant having “at least 99% sequence identity” to the polypeptide.

A nucleic acid or polynucleotide is “isolated” when it is at least partially or completely separated from other components, including but not limited to, for example, other proteins, nucleic acids, cells, etc. Similarly, a polypeptide, protein or peptide is “isolated” when it is at least partially or completely separated from other components, including but not limited to, for example, other proteins, nucleic acids, cells, etc. On a molar basis, an isolated species is more abundant than are other species in a composition. For example, an isolated species may comprise at least about 60%, about 65%, about 70%, about 75%, about 80%, about 85%, about 90%, about 91%, about 92%, about 93%, about 94%, about 95%, about 96%, about 97%, about 98%, about 99%, or about 100% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a nucleic acid or a protein sample, respectively, followed by visualization upon staining. If desired, a high-resolution technique, such as high performance liquid chromatography (HPLC) or a similar means can be utilized for purification of the material.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than in a starting composition.

The term “cleaning activity” refers to a cleaning performance achieved by a serine protease polypeptide, variant, or reference subtilisin under conditions prevailing during the proteolytic, hydrolyzing, cleaning, or other process of the disclosure. In some embodiments, cleaning performance of a serine protease or reference subtilisin may be determined by using various assays for cleaning one or more enzyme sensitive stain on an item or surface (e.g., a stain resulting from food, grass, blood, ink, milk, oil, and/or egg protein). Cleaning performance of one or more subtilisin variant described herein or reference subtilisin can be determined by subjecting the stain on the item or surface to standard wash condition(s) and assessing the degree to which the stain is removed by using various chromatographic, spectrophotometric, or other quantitative methodologies. Exemplary cleaning assays and methods are known in the art and include, but are not limited to those described in WO99/34011 and U.S. Pat. No. 6,605,458, as well as those cleaning assays and methods included in Examples 6 and 7 provided below.

As used herein, “surface property” can be used in reference to electrostatic charge, as well as properties such as the hydrophobicity and hydrophilicity exhibited by the surface of a protein.

The disclosure includes subtilisin variants having one or more modifications at a surface exposed amino acid. Surface modifications in the enzyme variants can be useful in a detergent composition by having a minimum performance index for wash performance, stability of the enzyme in detergent compositions and thermostability of the enzyme, while having at least one of these characteristics improved from a parent subtilisin enzyme. In some embodiments, the surface modification changes the hydrophobicity and/or charge of the amino acid at that position. Hydrophobicity can be determined using techniques known in the art, such as those described in White and Wimley (White, S. H. and Wimley, W. C. (1999) Annu. Rev. Biophys. Biomol. Struct. 28:319-65). Net charge of an amino acid at a pH of interest can be calculated using the pKₐ values of titratable chemical groups in amino acids, such as those described in Hass and Mulder (Hass, M. A. S and Mulder, F. A. A (2015) Annu. Rev. Biophys. 44:53-75). A subtilisin variant having a net charge of −4 to +2 at pH 8 relative to the subtilisin having the amino acid sequence of SEQ ID NO: 1 may find benefit in laundry cleaning applications.

The term “effective amount” of one or more subtilisin variant described herein or reference subtilisin refers to the amount of protease that achieves a desired level of enzymatic activity in a specific cleaning composition. Such effective amounts are readily ascertained by one of ordinary skill in the art and are based on many factors, such as the particular protease used, the cleaning application, the specific composition of the cleaning composition, and whether a liquid or dry (e.g., granular, tablet, bar) composition is required, etc.

The term “adjunct material” refers to any liquid, solid, or gaseous material included in cleaning composition other than one or more subtilisin variant described herein, or recombinant polypeptide or active fragment thereof. In some embodiments, the cleaning compositions of the present disclosure include one or more cleaning adjunct materials. Each cleaning adjunct material is typically selected depending on the particular type and form of cleaning composition (e.g., liquid, granule, powder, bar, paste, spray, tablet, gel, foam, or other composition). Preferably, each cleaning adjunct material is compatible with the protease enzyme used in the composition.

Cleaning compositions and cleaning formulations include any composition that is suited for cleaning, bleaching, disinfecting, and/or sterilizing any object, item, and/or surface. Such compositions and formulations include, but are not limited to, for example, liquid and/or solid compositions, including cleaning or detergent compositions (e.g., liquid, tablet, gel, bar, granule, and/or solid laundry cleaning or detergent compositions and fine fabric detergent compositions; hard surface cleaning compositions; medical instrument cleaning compositions and formulations, such as for glass, wood, ceramic and metal counter tops and windows; carpet cleaners; oven cleaners; fabric fresheners; fabric softeners; and textile, laundry booster cleaning or detergent compositions, laundry additive cleaning compositions, and laundry pre-spotter cleaning compositions; dishwashing compositions, including hand or manual dishwashing compositions (e.g., “hand” or “manual” dishwashing detergents) and automatic dishwashing compositions (e.g., “automatic dishwashing detergents”). Single dosage unit forms also find use with the present invention, including but not limited to pills, tablets, gelcaps, or other single dosage units such as pre-measured powders or liquids.

Cleaning composition or cleaning formulations, as used herein, include, unless otherwise indicated, granular or powder-form all-purpose or heavy-duty washing agents, especially cleaning detergents; liquid, granular, gel, solid, tablet, paste, or unit dosage form all-purpose washing agents, especially the so-called heavy-duty liquid (HDL) detergent or heavy-duty dry (HDD) detergent types; liquid fine-fabric detergents; hand or manual dishwashing agents, including those of the high-foaming type; hand or manual dishwashing, automatic dishwashing, or dishware or tableware washing agents, including the various tablet, powder, solid, granular, liquid, gel, and rinse-aid types for household and institutional use; liquid cleaning and disinfecting agents, including antibacterial hand-wash types, cleaning bars, mouthwashes, denture cleaners, car shampoos, carpet shampoos, bathroom cleaners; hair shampoos and/or hair-rinses for humans and other animals; shower gels and foam baths and metal cleaners; as well as cleaning auxiliaries, such as bleach additives and “stain-stick” or pre-treat types. In some embodiments, granular compositions are in “compact” form; in some embodiments, liquid compositions are in a “concentrated” form.

The term “detergent composition” or “detergent formulation” is used in reference to a composition intended for use in a wash medium for the cleaning of soiled or dirty objects, including particular fabric and/or non-fabric objects or items. In some embodiments, the detergents of the disclosure comprise one or more subtilisin variant described herein and, in addition, one or more surfactants, transferase(s), hydrolytic enzymes, oxido reductases, builders (e.g., a builder salt), bleaching agents, bleach activators, bluing agents, fluorescent dyes, caking inhibitors, masking agents, enzyme stabilizers, calcium, enzyme activators, antioxidants, and/or solubilizers. In some instances, a builder salt is a mixture of a silicate salt and a phosphate salt, preferably with more silicate (e.g., sodium metasilicate) than phosphate (e.g., sodium tripolyphosphate). Some embodiments are directed to cleaning compositions or detergent compositions that do not contain any phosphate (e.g., phosphate salt or phosphate builder). Detergent compositions may also contain biological ingredients, such as one or one or more microorganisms or microbes or microbial extracts (as described in WO2018060475 and U.S. Pat. No. 10,968,556). Microorganisms may be used as the only biologically active ingredient, but they may also be used in conjunction with one or more of the enzymes described herein. A bacillus strain having the deposit accession number PTA-7543, for example, may be used to reduce malodor as described in WO 2012/112718. Other purposes could include in-situ production of desirable bio-logical compounds, or inoculation/population of a locus with the microorganism(s) to competitively prevent other non-desirable microorganisms form populating the same locus (competitive exclusion).

The phrase “composition(s) substantially-free of boron” or “detergent(s) substantially-free of boron” refers to composition(s) or detergent(s), respectively, that contain trace amounts of boron, for example, less than about 1000 ppm (1 mg/kg or liter equals 1 ppm), less than about 100 ppm, less than about 50 ppm, less than about 10 ppm, or less than about 5 ppm, or less than about 1 ppm, perhaps from other compositions or detergent constituents.

The term “bleaching” refers to the treatment of a material (e.g., fabric, laundry, pulp, etc.) or surface for a sufficient length of time and/or under appropriate pH and/or temperature conditions to effect a brightening (i.e., whitening) and/or cleaning of the material. Examples of chemicals suitable for bleaching include, but are not limited to, for example, ClO₂, H₂O₂, peracids, NO₂, etc. Bleaching agents also include enzymatic bleaching agents such as perhydrolase and arylesterases. Another embodiment is directed to a composition comprising one or more subtilisin variant described herein, and one or more perhydrolase, such as, for example, is described in WO2005/056782, WO2007/106293, WO 2008/063400, WO2008/106214, and WO2008/106215.

The term “wash performance” of a protease (e.g., one or more subtilisin variant described herein, or recombinant polypeptide or active fragment thereof) refers to the contribution of one or more subtilisin variant described herein to washing that provides additional cleaning performance to the detergent as compared to the detergent without the addition of the one or more subtilisin variant described herein to the composition. Wash performance is compared under relevant washing conditions. In some test systems, other relevant factors, such as detergent composition, sud concentration, water hardness, washing mechanics, time, pH, and/or temperature, can be controlled in such a way that condition(s) typical for household application in a certain market segment (e.g., hand or manual dishwashing, automatic dishwashing, dishware cleaning, tableware cleaning, fabric cleaning, etc.) are imitated.

The phrase “relevant washing conditions” is used herein to indicate the conditions, particularly washing temperature, time, washing mechanics, sud concentration, type of detergent and water hardness, actually used in households in a hand dishwashing, automatic dishwashing, or laundry detergent market segment.

The term “dish wash” refers to both household and industrial dish washing and relates to both automatic dish washing (e.g. in a dishwashing machine) and manual dishwashing (e.g. by hand).

The term “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically about 17 to about 35% by weight of the total composition. In contrast, in compact compositions, the filler salt is present in amounts less than about 15% of the total composition. In some embodiments, the filler salt is present in amounts that do not exceed about 10%, or more preferably, about 5%, by weight of the composition. In some embodiments, the inorganic filler salts are selected from the alkali and alkaline-earth-metal salts of sulfates and chlorides. In some embodiments, the filler salt is sodium sulfate.

Disclosed herein is one or more subtilisin variant useful for cleaning applications and in methods of cleaning, as well as in a variety of industrial applications. Also disclosed herein is one or more isolated, recombinant, substantially pure, or non-naturally occurring subtilisin variant. In some embodiments, one or more subtilisin variant described herein is useful in cleaning applications and can be incorporated into cleaning compositions that are useful in methods of cleaning an item or a surface in need thereof, such as a laundry item or textile.

In one embodiment, subtilisin variants are provided, where the variant comprises two, three, four, or more amino acid substitutions at a position selected from the group consisting of 9, 17, 45, 68, 78, 86, 87, 96, 100, 103, 108, 115, 117, 127, 128, 129, 155, 161, 181, 202, 203, 217, 221,260, and 264 where the positions are numbered according to SEQ ID NO: 1, and where the variant has at least 60% identity to the amino acid sequence of SEQ ID NO: 1 or SEQ ID NO: 8.

In another embodiment, subtilisin variants are provided, where the variant comprises one, two, three, or four, or more amino acid substitutions selected from the group consisting of X9T, X17H, X45R, X68S, X78I, X86E, X87A, X96D, X100E, X100N, X103F, X103I, X108Q, X115L, X117R, X127S, X127T, X128K, X128P, X128R, X129Q, X155E, X161Q, X181E, X181Q, X202V, X203E, X203N, X217S, X221Q, X260W, and X264H, where the positions are numbered according to SEQ ID NO: 1, and where the variant has at least 75% identity to the amino acid sequence of SEQ ID NO: 1 or SEQ ID NO: 8.

In other embodiments, the subtilisin variants still further comprise additional substitutions selected from the group consisting of X24Q, X77N, X86D, X165Q, X184Q, X258D, and X258P, where the positions are numbered by correspondence with the amino acid sequence of SEQ ID NO: 1.

In one embodiment, subtilisin variants are provided, where the variant comprises X9T-X17H, X9T-X45R, X9T-X68S, X9T-X78I, X9T-X86E, X9T-X87A, X9T-X96D, X9T-X100E, X9T-X100N, X9T-X103F, X9T-X103I, X9T-X108Q, X9T-X115L, X9T-X117R, X9T-X127S, X9T-X127T, X9T-X128K, X9T-X128P, X9T-X128R, X9T-X129Q, X9T-X155E, X9T-X161Q, X9T-X181E, X9T-X181Q, X9T-X202V, X9T-X203E, X9T-X203N, X9T-X217S, X9T-X260W, X9T-X221Q, X9T-X264H, X17H-X45R, X17H-X68S, X17H-X78I, X17H-X86E, X17H-X87A, X17H-X96D, X17H-X100E, X17H-X100N, X17H-X103F, X17H-X103I, X17H-X108Q, X17H-X115L, X17H-X117R, X17H-X127S, X17H-X127T, X17H-X128K, X17H-X128P, X17H-X128R, X17H-X129Q, X17H-X155E, X17H-X161Q, X17H-X181E, X17H-X181Q, X17H-X202V, X17H-X203E, X17H-X203N, X17H-X217S, X17H-X260W, X17H-X221Q, X17H-X264H, X45R-X68S, X45R-X78I, X45R-X86E, X45R-X87A, X45R-X96D, X45R-X100E, X45R-X100N, X45R-X103F, X45R-X103I, X45R-X108Q, X45R-X115L, X45R-X117R, X45R-X127S, X45R-X127T, X45R-X128K, X45R-X128P, X45R-X128R, X45R-X129Q, X45R-X155E, X45R-X161Q, X45R-X181E, X45R-X181Q, X45R-X202V, X45R-X203E, X45R-X203N, X45R-X217S, X45R-X260W, X45R-X221Q, X45R-X264H, X68S-X78I, X68S-X86E, X68S-X87A, X68S-X96D, X68S-X100E, X68S-X100N, X68S-X103F, X68S-X103I, X68S-X108Q, X68S-X115L, X68S-X117R, X68S-X127S, X68S-X127T, X68S-X128K, X68S-X128P, X68S-X128R, X68S-X129Q, X68S-X155E, X68S-X161Q, X68S-X181E, X68S-X181Q, X68S-X202V, X68S-X203E, X68S-X203N, X68S-X217S, X68S-X260W, X68S-X221Q, X68S-X264H, X78I-X86E, X78I-X87A, X78I-X96D, X78I-X100E, X78I-X100N, X78I-X103F, X78I-X103I, X78I-X108Q, X78I-X115L, X78I-X117R, X78I-X127S, X78I-X127T, X78I-X128K, X78I-X128P, X78I-X128R, X78I-X129Q, X78I-X155E, X78I-X161Q, X78I-X181E, X78I-X181Q, X78I-X202V, X78I-X203E, X78I-X203N, X78I-X217S, X78I-X260W, X78I-X221Q, X78I-X264H, X86E-X87A, X86E-X96D, X86E-X100E, X86E-X100N, X86E-X103F, X86E-X103I, X86E-X108Q, X86E-X115L, X86E-X117R, X86E-X127S, X86E-X127T, X86E-X128K, X86E-X128P, X86E-X128R, X86E-X129Q, X86E-X155E, X86E-X161Q, X86E-X181E, X86E-X181Q, X86E-X202V, X86E-X203E, X86E-X203N, X86E-X217S, X86E-X260W, X86E-X221Q, X86E-X264H, X87A-X96D, X87A-X100E, X87A-X100N, X87A-X103F, X87A-X103I, X87A-X108Q, X87A-X115L, X87A-X117R, X87A-X127S, X87A-X127T, X87A-X128K, X87A-X128P, X87A-X128R, X87A-X129Q, X87A-X155E, X87A-X161Q, X87A-X181E, X87A-X181Q, X87A-X202V, X87A-X203E, X87A-X203N, X87A-X217S, X87A-X260W, X87A-X221Q, X87A-X264H, X96D-X100E, X96D-X100N, X96D-X103F, X96D-X103I, X96D-X108Q, X96D-X115L, X96D-X117R, X96D-X127S, X96D-X127T, X96D-X128K, X96D-X128P, X96D-X128R, X96D-X129Q, X96D-X155E, X96D-X161Q, X96D-X181E, X96D-X181Q, X96D-X202V, X96D-X203E, X96D-X203N, X96D-X217S, X96D-X260W, X96D-X221Q, X96D-X264H, X100E-X100N, X100E-X103F, X100E-X103I, X100E-X108Q, X100E-X115L, X100E-X117R, X100E-X127S, X100E-X127T, X100E-X128K, X100E-X128P, X100E-X128R, X100E-X129Q, X100E-X155E, X100E-X161Q, X100E-X181E, X100E-X181Q, X100E-X202V, X100E-X203E, X100E-X203N, X100E-X217S, X100E-X260W, X100E-X221Q, X100E-X264H, X100N-X103F, X100N-X103I, X100N-X108Q, X100N-X115L, X100N-X117R, X100N-X127S, X100N-X127T, X100N-X128K, X100N-X128P, X100N-X128R, X100N-X129Q, X100N-X155E, X100N-X161Q, X100N-X181E, X100N-X181Q, X100N-X202V, X100N-X203E, X100N-X203N, X100N-X217S, X100N-X260W, X100N-X221Q, X100N-X264H, X103F-X103I, X103F-X108Q, X103F-X115L, X103F-X117R, X103F-X127S, X103F-X127T, X103F-X128K, X103F-X128P, X103F-X128R, X103F-X129Q, X103F-X155E, X103F-X161Q, X103F-X181E, X103F-X181Q, X103F-X202V, X103F-X203E, X103F-X203N, X103F-X217S, X103F-X260W, X103F-X221Q, X103F-X264H, X103I-X108Q, X103I-X115L, X103I-X117R, X103I-X127S, X103I-X127T, X103I-X128K, X103I-X128P, X103I-X128R, X103I-X129Q, X103I-X155E, X103I-X161Q, X103I-X181E, X103I-X181Q, X103I-X202V, X103I-X203E, X103I-X203N, X103I-X217S, X103I-X260W, X103I-X221Q, X103I-X264H, X108Q-X115L, X108Q-X117R, X108Q-X127S, X108Q-X127T, X108Q-X128K, X108Q-X128P, X108Q-X128R, X108Q-X129Q, X108Q-X155E, X108Q-X161Q, X108Q-X181E, X108Q-X181Q, X108Q-X202V, X108Q-X203E, X108Q-X203N, X108Q-X217S, X108Q-X260W, X108Q-X221Q, X108Q-X264H, X115L-X117R, X115L-X127S, X115L-X127T, X115L-X128K, X115L-X128P, X115L-X128R, X115L-X129Q, X115L-X155E, X115L-X161Q, X115L-X181E, X115L-X181Q, X115L-X202V, X115L-X203E, X115L-X203N, X115L-X217S, X115L-X260W, X115L-X221Q, X115L-X264H, X117R-X127S, X117R-X127T, X117R-X128K, X117R-X128P, X117R-X128R, X117R-X129Q, X117R-X155E, X117R-X161Q, X117R-X181E, X117R-X181Q, X117R-X202V, X117R-X203E, X117R-X203N, X117R-X217S, X117R-X260W, X117R-X221Q, X117R-X264H, X127S-X127T, X127S-X128K, X127S-X128P, X127S-X128R, X127S-X129Q, X127S-X155E, X127S-X161Q, X127S-X181E, X127S-X181Q, X127S-X202V, X127S-X203E, X127S-X203N, X127S-X217S, X127S-X260W, X127S-X221Q, X127S-X264H, X127T-X128K, X127T-X128P, X127T-X128R, X127T-X129Q, X127T-X155E, X127T-X161Q, X127T-X181E, X127T-X181Q, X127T-X202V, X127T-X203E, X127T-X203N, X127T-X217S, X127T-X260W, X127T-X221Q, X127T-X264H, X128K-X128P, X128K-X128R, X128K-X129Q, X128K-X155E, X128K-X161Q, X128K-X181E, X128K-X181Q, X128K-X202V, X128K-X203E, X128K-X203N, X128K-X217S, X128K-X260W, X128K-X221Q, X128K-X264H, X128P-X128R, X128P-X129Q, X128P-X155E, X128P-X161Q, X128P-X181E, X128P-X181Q, X128P-X202V, X128P-X203E, X128P-X203N, X128P-X217S, X128P-X260W, X128P-X221Q, X128P-X264H, X128R-X129Q, X128R-X155E, X128R-X161Q, X128R-X181E, X128R-X181Q, X128R-X202V, X128R-X203E, X128R-X203N, X128R-X217S, X128R-X260W, X128R-X221Q, X128R-X264H, X129Q-X155E, X129Q-X161Q, X129Q-X181E, X129Q-X181Q, X129Q-X202V, X129Q-X203E, X129Q-X203N, X129Q-X217S, X129Q-X260W, X129Q-X221Q, X129Q-X264H, X155E-X161Q, X155E-X181E, X155E-X181Q, X155E-X202V, X155E-X203E, X155E-X203N, X155E-X217S, X155E-X260W, X155E-X221Q, X155E-X264H, X161Q-X181E, X161Q-X181Q, X161Q-X202V, X161Q-X203E, X161Q-X203N, X161Q-X217S, X161Q-X260W, X161Q-X221Q, X161Q-X264H, X181E-X181Q, X181E-X202V, X181E-X203E, X181E-X203N, X181E-X217S, X181E-X260W, X181E-X221Q, X181E-X264H, X181Q-X202V, X181Q-X203E, X181Q-X203N, X181Q-X217S, X181Q-X260W, X181Q-X221Q, X181Q-X264H, X202V-X203E, X202V-X203N, X202V-X217S, X202V-X260W, X202V-X221Q, X202V-X264H, X203E-X203N, X203E-X217S, X203E-X260W, X203E-X221Q, X203E-X264H, X203N-X217S, X203N-X260W, X203N-X221Q, X203N-X264H, X217S-X260W, X217S-X221Q, X217S-X264H, X260W-X221Q, X260W-X264H, and X221Q-X264H, where the positions are numbered according to SEQ ID NO: 1, and where the variant has at least 75% identity to the amino acid sequence of SEQ ID NO: 1 or SEQ ID NO: 8.

In another embodiment, subtilisin variants are provided, X017H-X096D-X127T; X017H-X096D-X103F-X202V; X127T-X128K-X129Q-X184Q; X009T-X103F-X202V-X203E; X096D-X100E-X127T-X202V; X087A-X155E-X165Q; X009T-X078I-X103F-X127T; X087A-X202V-X203E; X045R-X161Q-X181Q-X203E; X155E-X221Q; X078I-X103F; X096D-X100E-X127T-X217S; X127T-X128K-X129Q; X115L-X127T; X009T-X078I-X096D-X202V; X045R-X086E-X155E-X202V; X045R-X078I-X103F-X202V; X009T-X202V-X203E-X221Q; X202V-X203E-X221Q-X264H; X096D-X127T; X009T-X017H-X103F-X202V; X045R-X127T-X128P-X129Q; X100E-X127T-X202V; X202V-X221Q; X100N-X103I-X127T; X127T-X128K-X129Q-X217S; X127S-X128K-X129Q-X202V; X078I-X096D; X128K-X155E; X096D-X103F-X165Q; X086E-X077N-X078I; X045R-X078I-X086E-X202V; X096D-X100E-X127S; X078I-X127T-X128K-X129Q; X127S-X128K-X129Q; X017H-X086E-X202V-X203E; X161Q-X181Q-X202V-X203E; X045R-X096D-X100E; X045R-X103F-X127T-X202V; X096D-X100E-X117R-X127T; X087A-X165Q-X202V; X127T-X128P-X129Q; X087A-X221Q; X115L-X202V-X203E; X045R-X078I-X096D-X202V; X127T-X128R-X129Q-X165Q; X100E-X103I; X086E-X155E-X202V-X203E; X096D-X103I-X127T; X017H-X096D-X103F; X045R-X078I-X096D-X103F; X108Q-X127T; X078I-X086E-X202V-X203E; X078I-X103F-X127T-X202V; X181E-X202V-X203E-X258D; X096D-X202V-X203E; X096D-X103I-X127T-X221Q; X087A-X165Q-X202V-X203E; X009T-X127T-X128K-X129Q; X100E-X127T; X100N-X221Q; X127T-X128K-X129Q-X202V; X087A-X100N; X100E-X117R-X127T; X077N-X127T-X128K-X129Q; X017H-X096D-X127T-X202V; X096D-X202V-X221Q; X045R-X096D-X103F-X127T; X096D-X127T-X203E; X045R-X086E-X202V-X203E; X078I-X086E-X155E-X203E; X045R-X221Q; X017H-X096D-X103F-X127T; X017H-X103F-X127T-X202V; X078I-X202V-X221Q; X045R-X096D-X127T; X045R-X096D-X127T-X202V; X009T-X078I-X103F-X202V; X100N-X115L; X086E-X202V; X096D-X103F-X127T; X078I-X096D-X127T; X115L-X165Q-X202V; X078I-X086E; X045R-X078I-X096D-X127T; X009T-X096D-X127T; X127T-X128R-X129Q-X202V; X078I-X096D-X202V; X096D-X103I-X203E; X127S-X128P-X129Q-X202V; X096D-X127T-X217S; X045R-X078I; X024Q-X078I-X202V; X202V-X203N; X009T-X077N-X078I; X203E-X264H; X077N-X078I-X165Q-X202V; X045R-X077N-X165Q-X202V; X165Q-X202V-X203E-X258P; X009T-X078I-X202V; X045R-X127T; X077N-X078I-X165Q-X203E; X045R-X078I-X202V-X203E; X127T-X165Q-X202V-X203N; X202V-X264H; X009T-X202V-X203E; X077N-X202V-X203E; X202V-X203E-X217S; X103I-X202V; X078I-X203N; X009T-X017H-X078I-X165Q; X017H-X078I; X086D-X202V-X203E; X202V-X203E-X260W; X165Q-X202V-X203E-X217S;

X127T-X165Q-X202V-X203E; X009T-X017H-X078I-X127T; X045R-X155E; X009T-X017H-X078I-X202V; X009T-X078I; X086D-X155E-X202V-X203E; X009T-X017H-X202V-X203E; X024Q-X165Q-X202V-X203E; X009T-X017H-X077N-X202V; X017H-X202V-X203E; X009T-X127T-X202V-X203E; X103I-X203E; X078I-X155E-X202V-X203E; X096D-X100E-X103I; X017H-X078I-X127T-X202V; X009T-X096D; X128R-X155E; X127T-X128R; X078I-X202V; X009T-X202V; X017H-X202V; X009T-X203E; X078I-X165Q-X202V; X078I-X202V-X203E-X217S; and X077N-X078I-X221Q, where the positions are numbered according to SEQ ID NO: 1, and where the variant has at least 75% identity to the amino acid sequence of SEQ ID NO: 1 or SEQ ID NO: 8.

In another embodiment, subtilisin variants are provided, where the variant comprises X221Q; X100N; X115L; and X087A, where the positions are numbered according to SEQ ID NO: 1, and where the variant has at least 75% identity to the amino acid sequence of SEQ ID NO: 1 or SEQ ID NO: 8.

In another embodiment, subtilisin variants are provided, where the variant comprises Q017H-N096D-G127T; Q017H-N096D-Y103F-A202V; G127T-A128K-S129Q-N184Q; P009T-Y103F-A202V-G203E; N096D-S100E-G127T-A202V; V087A-S155E-G165Q; P009T-T078I-Y103F-G127T; V087A-A202V-G203E; V045R-T161Q-S181Q-G203E; S155E-M221Q; T078I-Y103F; N096D-S100E-G127T-N217S; G127T-A128K-S129Q; T115L-G127T; P009T-T078I-N096D-A202V; V045R-S086E-S155E-A202V; V045R-T078I-Y103F-A202V; P009T-A202V-G203E-M221Q; A202V-G203E-M221Q-K264H; N096D-G127T; P009T-Q017H-Y103F-A202V; V045R-G127T-A128P-S129Q; S100E-G127T-A202V; A202V-M221Q; S100N-Y103I-G127T; G127T-A128K-S129Q-N217S; G127S-A128K-S129Q-A202V; T078I-N096D; A128K-S155E; N096D-Y103F-G165Q; S086E-T077N-T078I; V045R-T078I-S086E-A202V; N096D-S100E-G127S; T078I-G127T-A128K-S129Q; G127S-A128K-S129Q; Q017H-S086E-A202V-G203E; T161Q-S181Q-A202V-G203E; V045R-N096D-S100E; V045R-Y103F-G127T-A202V; N096D-S100E-G117R-G127T; V087A-G165Q-A202V; G127T-A128P-S129Q; V087A-M221Q; T115L-A202V-G203E; V045R-T078I-N096D-A202V; G127T-A128R-S129Q-G165Q; S100E-Y103I; S086E-S155E-A202V-G203E; N096D-Y103I-G127T; Q017H-N096D-Y103F; V045R-T078I-N096D-Y103F; S108Q-G127T; T078I-S086E-A202V-G203E; T078I-Y103F-G127T-A202V; S181E-A202V-G203E-S258D; N096D-A202V-G203E; N096D-Y103I-G127T-M221Q; V087A-G165Q-A202V-G203E; P009T-G127T-A128K-S129Q; S100E-G127T; S100N-M221Q; G127T-A128K-S129Q-A202V; V087A-S100N; S100E-G117R-G127T; T077N-G127T-A128K-S129Q; Q017H-N096D-G127T-A202V; N096D-A202V-M221Q; V045R-N096D-Y103F-G127T; N096D-G127T-G203E; V045R-S086E-A202V-G203E; T078I-S086E-S155E-G203E; V045R-M221Q; Q017H-N096D-Y103F-G127T; Q017H-Y103F-G127T-A202V; T078I-A202V-M221Q; V045R-N096D-G127T; V045R-N096D-G127T-A202V; P009T-T078I-Y103F-A202V; S100N-T115L; S086E-A202V; N096D-Y103F-G127T; T078I-N096D-G127T; T115L-G165Q-A202V; T078I-S086E; V045R-T078I-N096D-G127T; P009T-N096D-G127T; G127T-A128R-S129Q-A202V; T078I-N096D-A202V; N096D-Y103I-G203E; G127S-A128P-S129Q-A202V; N096D-G127T-N217S; and T077N-T078I-M221Q, where the positions are numbered according to SEQ ID NO: 1, and where the variant has at least 75% identity to the amino acid sequence of SEQ ID NO: 1 or SEQ ID NO: 8.

In another embodiment, subtilisin variants are provided, where the variant comprises M221Q; S100N; T115L; or S087A, where the positions are numbered according to SEQ ID NO: 1, and where the variant has at least 75% identity to the amino acid sequence of SEQ ID NO: 1 or SEQ ID NO: 8.

In other embodiments, the subtilisin variants provided herein comprises an amino acid sequence having at least 75% sequence identity to the amino acid sequence of SEQ ID NO: 1 and has at least two substitutions selected from the group consisting of X9T, X17H, X45R, X68S, X78I, X86E, X87A, X96D, X100E, X100N, X103F, X103I, X108Q, X115L, X117R, X127S, X127T, X128K, X128P, X128R, X129Q, X155E, X161Q, X181E, X181Q, X202V, X203E, X203N, X217S, X221Q X260W, and X264H, wherein the positions are numbered according to the amino acid sequence of SEQ ID NO: 1, and wherein the variant has a net charge of −4 to +2 at pH8 relative to the subtilisin having the amino acid sequence of SEQ ID NO: 1. In yet additional embodiments, such subtilisin variants may further comprise one or more substitutions selected from the group consisting of X24Q, X77N, X86D, X165Q, X184Q, X258D, and X258P, wherein the positions are numbered according to the amino acid sequence of SEQ ID NO: 1.

In other embodiments, the subtilisin variants provided herein comprise a set of substitutions selected from the group consisting of those variants listed in Tables 3, 4, 5, 6, 7, 8 and 15, wherein the positions are numbered according to SEQ ID NO: 1, and wherein the variant has at least 75% identity to the amino acid sequence of SEQ ID NO: 1 or SEQ ID NO: 8.

Another embodiment is directed to one or more subtilisin variant described herein with the proviso that one or more substitutions is non-naturally occurring. Yet an even still further embodiment is directed to one or more subtilisin variant described herein wherein said variant (i) is derived from a B. licheniformis subtilisin; (ii) is isolated; (iii) has proteolytic activity; or (iv) comprises a combination of (i) to (iii). Still yet another embodiment is directed to one or more subtilisin variant described herein, wherein said variant is derived from a parent or reference polypeptide with (i) 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO:1; or (ii) 100% amino acid sequence identity to the amino acid sequence of SEQ ID NO:1. In still another embodiment the parent comprises the amino acid sequence of SEQ ID NO: 1. An even further embodiment is directed to one or more subtilisin variant described herein, wherein said variant comprises an amino acid sequence with (i) 60%, 65%, 70%, 75%, 80%, 85%, 86%, 87%, 88%, 89%, 90%, 91%, 92%, 93%, 94%, 95%, 96%, 97%, 98%, 99% or less than 100% amino acid sequence identity to the amino acid sequence of SEQ ID NO:1.

In an even still further embodiment, one or more subtilisin variant described herein has one or more improved property when compared to a reference or parent subtilisin; wherein the improved property is selected from improved cleaning performance in detergents, improved stability in detergent or buffer, and improved aged cleaning performance, and combinations thereof. Aged cleaning performance refers to the difference in stain removal measured for a sample of aged test sample (where the enzyme is pre-incubated in detergent for an extended period of time such as 3-8 weeks at an elevated temperature such as 37° C.) compared to the ‘fresh’ stain cleaning for the same enzyme (no pre-incubation). Thus, an enzyme with improved aged cleaning performance displays a smaller difference between the aged and freshly prepared samples when compared to the same evaluation carried out with a reference/parent enzyme.

In another embodiment, parent subtilisin comprises an amino acid sequence of SEQ ID NO: 1. In another embodiment, the parent subtilisin is a polypeptide having the amino acid sequence of SEQ ID NO:1 or SEQ ID NO: 8. In yet another embodiment, the improved property is (i) an improved cleaning performance in detergent, wherein said variant has an improved cleaning performance on stains selected from the group consisting of blood/milk/ink on woven cotton (CS-05) stains, blood aged on cotton (CS-01) stains, chocolate rice pudding, aged on cotton (CS-100) stains, full egg with carbon black, aged on cotton (C-S-39) stains, chocolate soymilk drink, aged on cotton (C-S-45) stains, grass on cotton (CS-07) stains, chocolate milk with carbon black on cotton (C-03) stains, milk with carbon black on cotton (C-11) stains, blood/milk/ink on polycotton (EMPA 116) stains, blood, aged on polyester/cotton (KCS-01) stains, and any one combination thereof, compared to a parent subtilisin; and/or (ii) improved stability, where the variant has a greater residual activity compared to the parent or reference subtilisin. In still yet another embodiment, the cleaning performance in detergent is measured in accordance with the cleaning performance assays of Example 2; and/or the stability is measured in accordance with the detergent stability assay of Example 2. In one embodiment, the improved property is an improved cleaning performance at low temperature (such as 20° C.) in detergent, wherein said variant has an improved cleaning performance at low temperature on stains selected from the group consisting of blood aged on cotton (CS-01) stains, chocolate rice pudding, aged on cotton (CS-100) stains, full egg with carbon black, aged on cotton (C-S-39) stains, chocolate soymilk drink, aged on cotton (C-S-45) stains, grass on cotton (CS-07) stains, blood/milk/ink on polycotton (EMPA 116) stain, and any one combination thereof, compared to a parent subtilisin. In one embodiment, the improved property is an improved cleaning performance at an elevated temperature (such as 37° C.) in detergent, wherein said variant has an improved cleaning performance at an elevated temperature on stains selected from the group consisting of chocolate milk with carbon black on cotton (C-03) stains, blood/milk/ink on woven cotton (CS-05) stains, milk with carbon black on cotton (C-11) stains blood aged on cotton (CS-01) stains, chocolate rice pudding, grass on cotton (CS-07) stains, aged on cotton (CS-100) stains, full egg with carbon black, aged on cotton (C-S-39) stains, blood/milk/ink on polycotton (EMPA 116) stains, blood aged on polyester/cotton (KCS-01) stains, and any one combination thereof, compared to a parent subtilisin. The term “enhanced stability” or “improved stability” in the context of an oxidation, chelator, denaturant, surfactant, thermal and/or pH stable protease refers to a higher retained proteolytic activity of a subtilisin variant over time as compared to a reference or parent subtilisin protease, for example, a wild-type protease or parent protease, such as SEQ ID NO: 1 or SEQ ID NO: 8. Autolysis has been identified as one mode of subtilisin activity loss in liquid detergents. (Stoner et al., 2004 Protease autolysis in heavy-duty liquid detergent formulations: effects of thermodynamic stabilizers and protease inhibitors, Enzyme and Microbial Technology 34:114-125.).

The terms “thermally stable” and “thermostable” and “thermostability” with regard to a protease variant refer to a protease that retains a greater amount of residual activity when compared to the parent or reference protease after exposure to altered temperatures over a given period of time under conditions (or “stress conditions”) prevailing during proteolytic, hydrolysing, cleaning or other process. Residual activity is the amount of activity remaining after the test compared to the initial activity of the sample and can be reported as a percentage e.g. % remaining activity. “Altered temperatures” encompass increased or decreased temperatures. In some embodiments, the variant proteases provided herein retain at least about 5%, about 10%, about 20%, about 30%, about 40%, about 50%, about 60%, about 70%, about 80%, about 85%, about 90%, about 92%, about 95%, about 96%, about 97%, about 98%, or about 99% proteolytic activity after exposure to temperatures of 40° C. to 80° C., over a given time period, for example, at least about 5 minutes, at least about 20 minutes, at least about 60 minutes, about 90 minutes, about 120 minutes, about 180 minutes, about 240 minutes, about 300 minutes, about 360 minutes, about 420 minutes, about 480 minutes, about 540 minutes, about 600 minutes, about 660 minutes, about 720 minutes, about 780 minutes, about 840 minutes, about 900 minutes, about 960 minutes, about 1020 minutes, about 1080 minutes, about 1140 minutes, or about 1200 minutes. In some embodiments, the variant subtilisins provided herein have a residual activity that is greater than that of the parent or reference protease using the method set forth in Example 2. In some embodiments, the variant subtilisins provided herein have at least a 10% improved residual activity compared to the parent subtilisin when measured after 20 minutes at 43-63 degrees Celsius in a liquid detergent.

The subtilisin variants provided herein may be used in the production of various compositions, such as enzyme compositions and cleaning or detergent compositions. An enzyme composition comprises a subtilisin variant as provided herein. The enzyme composition can be in any form, such as granule, liquid formulations, or enzyme slurries.

Enzyme granules may be made by, e.g., rotary atomization, wet granulation, dry granulation, spray drying, disc granulation, extrusion, pan coating, spheronization, drum granulation, fluid-bed agglomeration, high-shear granulation, fluid-bed spray coating, crystallization, precipitation, emulsion gelation, spinning disc atomization and other casting approaches, and prilling processes. The core of the granule may be the granule itself or the inner nucleus of a layered granule.

The core may comprise one or more water soluble or dispersible agent(s), including but not limited to, sodium sulfate, sodium chloride, magnesium sulfate, zinc sulfate, and ammonium sulfate), citric acid, sugars (e.g., sucrose, lactose, glucose, granulated sucrose, maltodextrin and fructose), plasticizers (e.g., polyols, urea, dibutyl phthalate, and dimethyl phthalate), fibrous material (e.g., cellulose and cellulose derivatives such as hydroxyl-propyl-methyl cellulose, carboxy-methyl cellulose, and hydroxyl-ethyl cellulose), phosphate, calcium, a protease inhibitor and combinations thereof. Suitable dispersible agents include, but are not limited to, clays, nonpareils (combinations of sugar and starch; e.g., starch-sucrose non-pareils-ASNP), talc, silicates, carboxymethyl cellulose, starch, and combinations thereof.

In some embodiments, the core comprises mainly sodium sulfate. In some embodiments, the core consists essentially of sodium sulfate. In a particular embodiment, the core consists of only sodium sulfate.

In some embodiments, the core comprises a subtilisin variant as provided herein. In other embodiments, the core comprises one or more enzymes in addition to protease. In other embodiments, the core is inert and does not comprise enzymes.

In some embodiments, the core is an enzyme powder, including UFC containing an enzyme. The enzyme powder may be spray dried and may optionally be admixed with any of the water soluble or dispersible agents listed, herein. The enzyme may be, or may include, the protease to be stabilized, in which case the enzyme power should further include a stabilizer.

In some embodiments, the core is coated with at least one coating layer. In a particular embodiment, the core is coated with at least two coating layers. In another particular embodiment the core is coated with at least three coating layers. The materials used in the coating layer(s) can be suitable for use in cleaning and/or detergent compositions (see, e.g., US20100124586, WO9932595 and U.S. Pat. No. 5,324,649.

In some embodiments, a coating layer comprises one of more of the following materials: an inorganic salt (e.g., sodium sulfate, sodium chloride, magnesium sulfate, zinc sulfate, and ammonium sulfate), citric acid, a sugar (e.g., sucrose, lactose, glucose, and fructose), a plasticizer (e.g., polyols, urea, dibutyl phthalate, and dimethyl phthalate), fibrous material (e.g., cellulose and cellulose derivatives such as hydroxyl-propyl-methyl cellulose, carboxy-methyl cellulose, and hydroxyl-ethyl cellulose), clay, nonpareil (a combination of sugar and starch), silicate, carboxymethyl cellulose, phosphate, starch (e.g., corn starch), fats, oils (e.g., rapeseed oil, and paraffin oil), lipids, vinyl polymers, vinyl copolymers, polyvinyl alcohol (PVA), plasticizers (e.g., polyols, urea, dibutyl phthalate, dimethyl phthalate, and water), anti-agglomeration agents (e.g., talc, clays, amorphous silica, and titanium dioxide), anti-foam agents (such as FOAMBLAST 882® and EROL 6000KR), and talc. US20100124586, WO9932595, and U.S. Pat. No. 5,324,649 detail suitable components for the coating layers.

In some embodiments, the coating layer comprises sugars (e.g., sucrose, lactose, glucose, granulated sucrose, maltodextrin and fructose). In some embodiments, the coating layer comprises a polymer such as polyvinyl alcohol (PVA). Suitable PVA for incorporation in the coating layer(s) of the multi-layered granule include partially hydrolyzed, fully hydrolyzed and intermediately hydrolyzed having low to high degrees of viscosity. In some embodiments, the coating layer comprises an inorganic salt, such as sodium sulfate.

In some embodiments, at least one coating layer is an enzyme coating layer. In some embodiments, the core is coated with at least two enzyme layers. In another embodiment, the core is coated with at least three or more enzyme layers.

In some embodiments, the enzyme granules comprise a subtilisin variant as provided herein in combination with one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, dispersins, endo-beta-1,4-glucanases, endo-beta-mannanases, esterases, exo-mannanases, galactanases, glucoamylases, hemicellulases, hexosaminidase, hyaluronidases, keratinases, laccases, lactases, ligninases, lipases, lipoxygenases, lysozymes, mannanases, metalloproteases, nucleases (e.g. DNases and/or RNases), oxidases, oxidoreductases, pectate lyases, pectin acetyl esterases, pectinases, pentosanases, perhydrolases, peroxidases, phenoloxidases, phosphatases, phospholipases, phosphodiesterases, phytases, polygalacturonases, polyesterases, additional proteases, pullulanases, reductases, rhamnogalacturonases, beta-glucanases, tannases, transglutaminases, xanthan lyases, xylan acetyl-esterases, xylanases, xyloglucanases, xylosidases, and any combination or mixture thereof. Generally, at least one enzyme coating layer comprises at least one subtilisin variant as provided herein.

The above enzyme lists are examples only and are not meant to be exclusive. Any enzyme can be used in the granules described herein, including wild type, recombinant and variant enzymes of bacterial, fungal, yeast sources, and acid, neutral or alkaline enzymes.

Another embodiment is directed to a method of cleaning a surface, where the method comprises contacting a surface or an item in need of cleaning with an effective amount of one or more subtilisin variants as provided herein, or composition containing one or more subtilisin variants, as provided herein. In some embodiments, the surface or item in need of cleaning comprises a proteinaceous stain on the surface. In some embodiments, the surface or item in need of cleaning comprises a proteinaceous stain. The term “stain” comprises any type of soil on the surface of an item, such as a hard-surface item (e.g. a dish), a medical instrument or textile. In some embodiments, the stain is a proteinaceous stain. As used herein, a “proteinaceous stain” is a stain or soil that contains protein.

A further embodiment is directed to a method of cleaning a proteinaccous stain comprising contacting a surface or an item in need of cleaning with an effective amount of one or more subtilisin variants as provided herein or composition containing one or more subtilisin variants as provided herein.

Another embodiment is directed to a method of cleaning a stain selected from the group consisting of blood/milk/ink on woven cotton (CS-05) stains, blood aged on cotton (CS-01) stains, chocolate rice pudding, aged on cotton (CS-100) stains, full egg with carbon black, aged on cotton (C-S-39) stains, chocolate soymilk drink, aged on cotton (C-S-45) stains, grass on cotton (CS-07) stains, chocolate milk with carbon black on cotton (C-03) stains, milk with carbon black on cotton (C-11) stains, blood/milk/ink on polycotton (EMPA 116) stains, blood, aged on polyester/cotton (KCS-01) stains, and any one combination thereof, said method comprising contacting a surface or an item in need of cleaning with an effective amount of one or more subtilisin variants as provided herein or composition containing one or more such subtilisin variants.

Another embodiment is directed to a method of cleaning a stain at low temperature (such as 20° C. or 25° C.) comprising contacting a surface or an item in need of cleaning with an effective amount of one or more subtilisin variants as provided herein or composition containing one or more such subtilisin variants. In one embodiment the method is a method of cleaning a stain selected from the group consisting of blood aged on cotton (CS-01) stains, chocolate rice pudding, aged on cotton (CS-100) stains, full egg with carbon black, aged on cotton (C-S-39) stains, chocolate soymilk drink, aged on cotton (C-S-45) stains, grass on cotton (CS-07) stains, blood/milk/ink on polycotton (EMPA 116) stains, and any one combination thereof, at a low temperature (such as 20° C. or 25° C.) comprising contacting a surface or an item in need of cleaning with an effective amount of one or more subtilisin variants as provided herein or composition containing one or more such subtilisin variants. Another embodiment is directed to a method of cleaning a stain at an elevated temperature (such as 37° C.) comprising contacting a surface or an item in need of cleaning with an effective amount of one or more subtilisin variants as provided herein or composition containing one or more such subtilisin variants. In one embodiment the method is a method of cleaning a stain selected from the group consisting of chocolate milk with carbon black on cotton (C-03) stains, blood/milk/ink on woven cotton (CS-05) stains, milk with carbon black on cotton (C-11) stains blood aged on cotton (CS-01) stains, chocolate rice pudding, grass on cotton (CS-07) stains, aged on cotton (CS-100) stains, full egg with carbon black, aged on cotton (C-S-39) stains, blood/milk/ink on polycotton (EMPA 116) stains, blood aged on polyester/cotton (KCS-01) stains, and any one combination thereof, at an elevated temperature (such as 37° C.) comprising contacting a surface or an item in need of cleaning with an effective amount of one or more subtilisin variants as provided herein or composition containing one or more such subtilisin variants.

One or more subtilisin variant described herein can be subject to various changes, such as one or more amino acid insertion, deletion, and/or substitution, either conservative or non-conservative, including where such changes do not substantially alter the enzymatic activity of the variant. Similarly, a nucleic acid of the invention can also be subject to various changes, such as one or more substitution of one or more nucleotide in one or more codon such that a particular codon encodes the same or a different amino acid, resulting in either a silent variation (e.g., when the encoded amino acid is not altered by the nucleotide mutation) or non-silent variation; one or more deletion of one or more nucleotides (or codon) in the sequence; one or more addition or insertion of one or more nucleotides (or codon) in the sequence; and/or cleavage of, or one or more truncation, of one or more nucleotides (or codon) in the sequence. Many such changes in the nucleic acid sequence may not substantially alter the enzymatic activity of the resulting encoded polypeptide enzyme compared to the polypeptide enzyme encoded by the original nucleic acid sequence. A nucleic acid sequence described herein can also be modified to include one or more codon that provides for optimum expression in an expression system (e.g., bacterial expression system), while, if desired, said one or more codon still encodes the same amino acid(s).

Described herein is one or more isolated, non-naturally occurring, or recombinant polynucleotide comprising a nucleic acid sequence that encodes one or more subtilisin variant described herein, or recombinant polypeptide or active fragment thereof. One or more nucleic acid sequence described herein is useful in recombinant production (e.g., expression) of one or more subtilisin variant described herein, typically through expression of a plasmid expression vector comprising a sequence encoding the one or more subtilisin variant described herein or fragment thereof. One embodiment provides nucleic acids encoding one or more subtilisin variant described herein, wherein the variant is a mature form having proteolytic activity. In some embodiments, one or more subtilisin variant described herein is expressed recombinantly with a homologous pro-peptide sequence. In other embodiments, one or more subtilisin variant described herein is expressed recombinantly with a heterologous or native pro-peptide sequence (e.g., pro-peptide sequence from B. licheniformis (SEQ ID NO:4)).

One or more nucleic acid sequence described herein can be generated by using any suitable synthesis, manipulation, and/or isolation techniques, or combinations thereof. For example, one or more polynucleotide described herein may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods) to form essentially any desired continuous nucleic acid sequence. The synthesis of the one or more polynucleotide described herein can be also facilitated by any suitable method known in the art, including but not limited to chemical synthesis using the classical phosphoramidite method (See e.g., Beaucage et al. Tetrahedron Letters 22:1859-69 (1981)), or the method described in Matthes et al., EMBO J. 3:801-805 (1984) as is typically practiced in automated synthetic methods. One or more polynucleotide described herein can also be produced by using an automatic DNA synthesizer. Customized nucleic acids can be ordered from a variety of commercial sources (e.g., ATUM (DNA 2.0), Newark, CA, USA; Life Tech (GeneArt), Carlsbad, CA, USA; GenScript, Ontario, Canada; Base Clear B. V., Leiden, Netherlands; Integrated DNA Technologies, Skokie, IL, USA; Ginkgo Bioworks (Gen9), Boston, MA, USA; and Twist Bioscience, San Francisco, CA, USA). Other techniques for synthesizing nucleic acids and related principles are described by, for example, Itakura et al., Ann. Rev. Biochem. 53:323 (1984) and Itakura et al., Science 198:1056 (1984).

Recombinant DNA techniques useful in modification of nucleic acids are well known in the art, such as, for example, restriction endonuclease digestion, ligation, reverse transcription and cDNA production, and polymerase chain reaction (e.g., PCR). One or more polynucleotide described herein may also be obtained by screening cDNA libraries using one or more oligonucleotide probes that can hybridize to or PCR-amplify polynucleotides which encode one or more subtilisin variant described herein, or recombinant polypeptide or active fragment thereof. Procedures for screening and isolating cDNA clones and PCR amplification procedures are well known to those of skill in the art and described in standard references known to those skilled in the art. One or more polynucleotide described herein can be obtained by altering a naturally occurring polynucleotide backbone (e.g., that encodes one or more subtilisin variant described herein or reference subtilisin) by, for example, a known mutagenesis procedure (e.g., site-directed mutagenesis, site saturation mutagenesis, and in vitro recombination). A variety of methods are known in the art that are suitable for generating modified polynucleotides described herein that encode one or more subtilisin variant described herein, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches.

A further embodiment is directed to one or more vector comprising one or more subtilisin variant described herein (e.g., a polynucleotide encoding one or more subtilisin variant described herein); expression vectors or expression cassettes comprising one or more nucleic acid or polynucleotide sequence described herein; isolated, substantially pure, or recombinant DNA constructs comprising one or more nucleic acid or polynucleotide sequence described herein; isolated or recombinant cells comprising one or more polynucleotide sequence described herein; and compositions comprising one or more such vector, nucleic acid, expression vector, expression cassette, DNA construct, cell, cell culture, or any combination or mixtures thereof.

Some embodiments are directed to one or more recombinant cell comprising one or more vector (e.g., expression vector or DNA construct) described herein which comprises one or more nucleic acid or polynucleotide sequence described herein. Some such recombinant cells are transformed or transfected with such at least one vector, although other methods are available and known in the art. Such cells are typically referred to as host cells. Some such cells comprise bacterial cells, including, but not limited to Bacillus sp. cells, such as B. subtilis or B. licheniformis cells. Other embodiments are directed to recombinant cells (e.g., recombinant host cells) comprising one or more subtilisin described herein.

In some embodiments, one or more vector described herein is an expression vector or expression cassette comprising one or more polynucleotide sequence described herein operably linked to one or more additional nucleic acid segments required for efficient gene expression (e.g., a promoter operably linked to one or more polynucleotide sequence described herein). A vector may include a transcription terminator and/or a selection gene (e.g., an antibiotic resistant gene) that enables continuous cultural maintenance of plasmid-infected host cells by growth in antimicrobial-containing media.

An expression vector may be derived from plasmid or viral DNA, or in alternative embodiments, contains elements of both. Exemplary vectors include, but are not limited to pC194, pJH101, pE194, pHP13 (See, Harwood and Cutting [eds.], Chapter 3, Molecular Biological Methods for Bacillus, John Wiley & Sons (1990); suitable replicating plasmids for B. subtilis include those listed on p. 92). (See also, Perego, “Integrational Vectors for Genetic Manipulations in Bacillus subtilis”; Sonenshein et al., [eds.]; “Bacillus subtilis and Other Gram-Positive Bacteria: Biochemistry, Physiology and Molecular Genetics”, American Society for Microbiology, Washington, D.C. (1993), pp. 615-624); and p2JM103BBI).

For expression and production of a protein of interest (e.g., one or more subtilisin variant described herein) in a cell, one or more expression vector comprising one or more copy of a polynucleotide encoding one or more subtilisin variant described herein, and in some instances comprising multiple copies, is transformed into the cell under conditions suitable for expression of the variant. In some embodiments, a polynucleotide sequence encoding one or more subtilisin variant described herein (as well as other sequences included in the vector) is integrated into the genome of the host cell, while in other embodiments, a plasmid vector comprising a polynucleotide sequence encoding one or more subtilisin variant described herein remains as autonomous extra-chromosomal element within the cell. Some embodiments provide both extrachromosomal nucleic acid elements as well as incoming nucleotide sequences that are integrated into the host cell genome. The vectors described herein are useful for production of the one or more subtilisin variant described herein. In some embodiments, a polynucleotide construct encoding one or more subtilisin variant described herein is present on an integrating vector that enables the integration and optionally the amplification of the polynucleotide encoding the variant into the host chromosome. Examples of sites for integration are well known to those skilled in the art. In some embodiments, transcription of a polynucleotide encoding one or more subtilisin variant described herein is effectuated by a promoter that is the wild-type promoter for the parent subtilisin. In some other embodiments, the promoter is heterologous to the one or more subtilisin variant described herein, but is functional in the host cell. Exemplary promoters for use in bacterial host cells include, but are not limited to the amyE, amyQ, amyL, pstS, sacB, pSPAC, pAprE, pVeg, pHpaII, rrnIp2 promoters; the promoter of the B. stearothermophilus maltogenic amylase gene; the B. amyloliquefaciens (BAN) amylase gene; the B. subtilis alkaline protease gene; the B. clausii alkaline protease gene; the B. pumilus xylosidase gene; the B. thuringiensis cryIIIA; and the B. licheniformis alpha-amylase gene. Additional promoters include, but are not limited to the A4 promoter, as well as phage Lambda PR or PL promoters and the E. coli lac, trp or tac promoters.

One or more subtilisin variant described herein can be produced in host cells of any suitable microorganism, including bacteria and fungi. In some embodiments, one or more subtilisin variant described herein can be produced in Gram-positive bacteria. In some embodiments, the host cells are Bacillus spp., Streptomyces spp., Escherichia spp., Aspergillus spp., Trichoderma spp., Pseudomonas spp., Corynebacterium spp., Saccharomyces spp., or Pichia spp. In some embodiments, one or more subtilisin variant described herein is produced by Bacillus sp. host cells. Examples of Bacillus sp. host cells that find use in the production of the one or more subtilisin variant described herein include, but are not limited to B. licheniformis, B. gibsonii, B. lentus, B. subtilis, B. amyloliquefaciens, B. brevis, B. stearothermophilus, B. alkalophilus, B. coagulans, B. circulans, B. pumilus, B. thuringiensis, B. clausii, and B. megaterium, as well as other organisms within the genus Bacillus. In some embodiments, B. subtilis host cells are used to produce the variants described herein. U.S. Pat. Nos. 5,264,366 and 4,760,025 (RE 34,606) describe various Bacillus host strains that can be used to produce one or more subtilisin variant described herein, although other suitable strains can be used.

Several bacterial strains that can be used to produce one or more subtilisin variant described herein include non-recombinant (i.e., wild-type) Bacillus sp. strains, as well as variants of naturally-occurring strains and/or recombinant strains. In some embodiments, the host strain is a recombinant strain, wherein a polynucleotide encoding one or more subtilisin variant described herein has been introduced into the host. In some embodiments, the host strain is a B. subtilis host strain and particularly a recombinant B. subtilis host strain. Numerous B. subtilis strains are known, including, but not limited to for example, 1A6 (ATCC 39085), 168 (1A01), SB19, W23, Ts85, B637, PB 1753 through PB1758, PB3360, JH642, 1A243 (ATCC 39,087), ATCC 21332, ATCC 6051, MI113, DE100 (ATCC 39,094), GX4931, PBT 110, and PEP 211 strain (See e.g., Hoch et al., Genetics 73:215-228 (1973); See also, U.S. Pat. Nos. 4,450,235; 4,302,544; and EP 0134048). The use of B. subtilis as an expression host cell is well known in the art (See e.g., Palva et al., Gene 19:81-87 (1982); Fahnestock and Fischer, J. Bacteriol., 165:796-804 (1986); and Wang et al., Gene 69:39-47 (1988)).

In some embodiments, the Bacillus host cell is a Bacillus sp. that includes a mutation or deletion in at least one of the following genes: degU, degS, degR and degQ. In some embodiments, the mutation is in a degU gene, and in some embodiments the mutation is degU (Hy) 32 (See e.g., Msadek et al., J. Bacteriol. 172:824-834 (1990); and Olmos et al., Mol. Gen. Genet. 253:562-567 (1997)). In some embodiments, the Bacillus host comprises a mutation or deletion in scoC4 (See e.g., Caldwell et al., J. Bacteriol. 183:7329-7340 (2001)); spoIIE (See e.g., Arigoni et al., Mol. Microbiol. 31:1407-1415 (1999)); and/or oppA or other genes of the opp operon (See e.g., Perego et al., Mol. Microbiol. 5:173-185 (1991)). Indeed, it is contemplated that any mutation in the opp operon that causes the same phenotype as a mutation in the oppA gene will find use in some embodiments of the altered Bacillus strain described herein. In some embodiments, these mutations occur alone, while in other embodiments, combinations of mutations are present. In some embodiments, an altered Bacillus host cell strain that can be used to produce one or more subtilisin variant described herein is a Bacillus host strain that already includes a mutation in one or more of the above-mentioned genes. In addition, Bacillus sp. host cells that comprise mutation(s) and/or deletion(s) of endogenous protease genes find use. In some embodiments, the Bacillus host cell comprises a deletion of the aprE and the nprE genes. In other embodiments, the Bacillus sp. host cell comprises a deletion of 5 protease genes, while in other embodiments the Bacillus sp. host cell comprises a deletion of 9 protease genes (See e.g., US 2005/0202535).

Host cells are transformed with one or more nucleic acid sequence encoding one or more subtilisin variant described herein using any suitable method known in the art. Methods for introducing a nucleic acid (e.g., DNA) into Bacillus cells or E. coli cells utilizing plasmid DNA constructs or vectors and transforming such plasmid DNA constructs or vectors into such cells are well known. In some embodiments, the plasmids are subsequently isolated from E. coli cells and transformed into Bacillus cells. However, it is not essential to use intervening microorganisms such as E. coli, and in some embodiments, a DNA construct or vector is directly introduced into a Bacillus host.

Exemplary methods for introducing one or more nucleic acid sequence described herein into Bacillus cells are described in, for example, Ferrari et al., “Genetics,” in Harwood et al. [eds.], Bacillus, Plenum Publishing Corp. (1989), pp. 57-72; Saunders et al., J. Bacteriol. 157:718-726 (1984); Hoch et al., J. Bacteriol. 93:1925-1937 (1967); Mann et al., Current Microbiol. 13:131-135 (1986); Holubova, Folia Microbiol. 30:97 (1985); Chang et al., Mol. Gen. Genet. 168:11-115 (1979); Vorobjeva et al., FEMS Microbiol. Lett. 7:261-263 (1980); Smith et al., Appl. Env. Microbiol. 51:634 (1986); Fisher et al., Arch. Microbiol. 139:213-217 (1981); and McDonald, J. Gen. Microbiol. 130:203 (1984)). Indeed, such methods as transformation, including protoplast transformation and transfection, transduction, and protoplast fusion are well known and suited for use herein. Methods known in the art to transform Bacillus cells include such methods as plasmid marker rescue transformation, which involves the uptake of a donor plasmid by competent cells carrying a partially homologous resident plasmid (See, Contente et al., Plasmid 2:555-571 (1979); Haima et al., Mol. Gen. Genet. 223:185-191 (1990); Weinrauch et al., J. Bacteriol. 154:1077-1087 (1983); and Weinrauch et al., J. Bacteriol. 169:1205-1211 (1987)). In this method, the incoming donor plasmid recombines with the homologous region of the resident “helper” plasmid in a process that mimics chromosomal transformation.

In addition to commonly used methods, in some embodiments, host cells are directly transformed with a DNA construct or vector comprising a nucleic acid encoding one or more subtilisin variant described herein (i.e., an intermediate cell is not used to amplify, or otherwise process, the DNA construct or vector prior to introduction into the host cell). Introduction of a DNA construct or vector described herein into the host cell includes those physical and chemical methods known in the art to introduce a nucleic acid sequence (e.g., DNA sequence) into a host cell without insertion into the host genome. Such methods include, but are not limited to calcium chloride precipitation, electroporation, naked DNA, and liposomes. In additional embodiments, DNA constructs or vector are co-transformed with a plasmid, without being inserted into the plasmid. In further embodiments, a selective marker is deleted from the altered Bacillus strain by methods known in the art (See, Stahl et al., J. Bacteriol. 158:411-418 (1984); and Palmeros et al., Gene 247:255-264 (2000)).

In some embodiments, the transformed cells are cultured in conventional nutrient media. The suitable specific culture conditions, such as temperature, pH and the like are known to those skilled in the art and are well described in the scientific literature. Some embodiments provide a culture (e.g., cell culture) comprising one or more subtilisin variant or nucleic acid sequence described herein.

In some embodiments, host cells transformed with one or more polynucleotide sequence encoding one or more subtilisin variant described herein are cultured in a suitable nutrient medium under conditions permitting the expression of the variant, after which the resulting variant is recovered from the culture. In some embodiments, the variant produced by the cells is recovered from the culture medium by conventional procedures, including, but not limited to, for example, separating the host cells from the medium by centrifugation or filtration, precipitating the proteinaceous components of the supernatant or filtrate by means of a salt (e.g., ammonium sulfate), and chromatographic purification (e.g., ion exchange, gel filtration, affinity, etc.).

In some embodiments, one or more subtilisin variant produced by a recombinant host cell is secreted into the culture medium. A nucleic acid sequence that encodes a purification facilitating domain may be used to facilitate purification of the variant. A vector or DNA construct comprising a polynucleotide sequence encoding one or more subtilisin variant described herein may further comprise a nucleic acid sequence encoding a purification facilitating domain to facilitate purification of the variant (See e.g., Kroll et al., DNA Cell Biol. 12:441-53 (1993)). Such purification facilitating domains include, but are not limited to, for example, metal chelating peptides such as histidine-tryptophan modules that allow purification on immobilized metals (Sec, Porath, Protein Expr. Purif. 3:263-281 [1992]), protein A domains that allow purification on immobilized immunoglobulin, and the domain utilized in the FLAGS extension/affinity purification system. The inclusion of a cleavable linker sequence such as Factor XA or enterokinase (e.g., sequences available from Invitrogen, San Diego, CA) between the purification domain and the heterologous protein also find use to facilitate purification.

The present variant proteins can be produced in host cells, for example, by secretion or intracellular expression, using methods well-known in the art. Fermentation, separation, and concentration techniques are well known in the art and conventional methods can be used to prepare a concentrated, enzyme-containing solution. Host cells may be further processed, such as to release enzyme or to improve cell separation, for example by heating or by changing pH or salt content or by treating with enzymes including hen egg white lysozyme, T4 lysozyme, or enzymes described in WO2022047149. For production scale recovery, variant polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

A variety of methods can be used to determine the level of production of one or more mature subtilisin variant described herein in a host cell. Such methods include, but are not limited to, for example, methods that utilize either polyclonal or monoclonal antibodies specific for the protease. Exemplary methods include, but are not limited to enzyme-linked immunosorbent assays (ELISA), radioimmunoassays (RIA), fluorescent immunoassays (FIA), and fluorescent activated cell sorting (FACS). These and other assays are well known in the art (See e.g., Maddox et al., J. Exp. Med. 158:1211 (1983)).

Some other embodiments provide methods for making or producing one or more mature subtilisin variant described herein. A mature subtilisin variant does not include a signal peptide or a propeptide sequence. Some methods comprise making or producing one or more subtilisin variant described herein in a recombinant bacterial host cell, such as for example, a Bacillus sp. cell (e.g., a B. subtilis cell). Other embodiments provide a method of producing one or more subtilisin variant described herein, wherein the method comprises cultivating a recombinant host cell comprising a recombinant expression vector comprising a nucleic acid sequence encoding one or more subtilisin variant described herein under conditions conducive to the production of the variant. Some such methods further comprise recovering the variant from the culture.

Further embodiments provide methods of producing one or more subtilisin variant described herein, wherein the methods comprise: (a) introducing a recombinant expression vector comprising a nucleic acid encoding the variant into a population of cells (e.g., bacterial cells, such as B. subtilis cells); and (b) culturing the cells in a culture medium under conditions conducive to produce the variant encoded by the expression vector. Some such methods further comprise: (c) isolating the variant from the cells or from the culture medium.

A further embodiment is directed to a method of improving the cleaning performance or stability of a subtilisin comprising modifying a subtilisin to include one or more substitutions, or combination of substitutions, as provided herein.

Unless otherwise noted, all component or composition levels provided herein are made in reference to the active level of that component or composition, and are exclusive of impurities, for example, residual solvents or by-products, which may be present in commercially available sources. Enzyme components weights are based on total active protein. All percentages and ratios are calculated by weight unless otherwise indicated. All percentages and ratios are calculated based on the total composition unless otherwise indicated. Compositions described herein include cleaning compositions, such as detergent compositions. In the exemplified detergent compositions, the enzyme levels are expressed by pure enzyme by weight of the total composition and unless otherwise specified, the detergent ingredients are expressed by weight of the total compositions.

In one embodiment, one or more subtilisin variant described herein is useful in cleaning applications, such as, for example, but not limited to, cleaning dishware or tableware items, fabrics, medical instruments and items having hard surfaces (e.g., the hard surface of a table, table top, wall, furniture item, floor, and ceiling). In other embodiments, one or more subtilisin variant described herein is useful in disinfecting applications, such as, for example, but not limited to, disinfecting an automatic dishwashing or laundry machine. In one embodiment, the cleaning composition is a cleaning composition comprising one or more subtilisin variant described herein wherein the cleaning composition is a composition selected from the group consisting of a laundry detergent, a fabric softening detergent, a dishwashing detergent (e.g., automatic or hand dishwashing detergents), a hard-surface cleaning detergent, and a medical instrument cleaning composition.

Another embodiment is directed to a composition comprising one or more subtilisin variant described herein. In some embodiments, the composition is a cleaning composition. In other embodiments, the composition is a detergent composition. In yet other embodiments, the composition is selected from a laundry detergent composition, an automatic dishwashing (ADW) composition, a hand (manual) dishwashing detergent composition, a hard surface cleaning composition, an eyeglass cleaning composition, a medical instrument cleaning composition, a disinfectant (e.g., malodor or microbial) composition, and a personal care cleaning composition. In still other embodiments, the composition is a laundry detergent composition, an ADW composition, or a hand (manual) dishwashing detergent composition. Even still further embodiments are directed to fabric cleaning compositions, while other embodiments are directed to non-fabric cleaning compositions. In some embodiments, the cleaning composition is boron-free. In other embodiments, the cleaning composition is phosphate-free. In still other embodiments, the composition comprises one or more subtilisin variant described herein and one or more of an excipient, adjunct material, and/or additional enzyme.

In another embodiment, the disclosure provides detergent compositions (e.g. ADW compositions) comprising a surfactant and at least one subtilisin variant as provided herein. Such compositions may further comprise one or more of an excipient, adjunct material, and/or additional enzyme.

In yet still a further embodiment, the composition described herein contains phosphate, is phosphate-free, contains boron, is boron-free, or combinations thereof. In other embodiments, the composition is a boron-free composition. In some embodiments, a boron-free composition is a composition to which a borate stabilizer has not been added. In another embodiment, a boron-free composition is a composition that contains less than 5.5% boron. In a still further embodiment, a boron-free composition is a composition that contains less than 4.5% boron. In yet still another embodiment, a boron-free composition is a composition that contains less than 3.5% boron. In yet still a further embodiment, a boron-free composition is a composition that contains less than 2.5% boron. In even further embodiments, a boron-free composition is a composition that contains less than 1.5% boron. In another embodiment, a boron-free composition is a composition that contains less than 1.0% boron. In still further embodiments, a boron-free composition is a composition that contains less than 0.5% boron. In other embodiments, the composition is a composition free or substantially-free of enzyme stabilizers or peptide inhibitors.

In another embodiment, one or more composition described herein is in a form selected from gel, tablet, powder, granular, solid, liquid, unit dose, and combinations thereof. In yet another embodiment, one or more composition described herein is in a form selected from a low water compact formula, low water HDL or Unit Dose (UD), or high water formula or HDL. In some embodiments, the cleaning composition described herein is in a unit dose form. In other embodiments, the unit does form is selected from pills, tablets, capsules, gelcaps, sachets, pouches, multi-compartment pouches, and pre-measured powders or liquids. In some embodiments, the unit dose format is designed to provide controlled release of the ingredients within a multi-compartment pouch (or other unit dose format). Suitable unit dose and controlled release formats are described, for example, in EP 2100949; WO 02/102955; U.S. Pat. Nos. 4,765,916; 4,972,017; and WO 04/111178. In some embodiments, the unit dose form is a tablet or powder contained in a water-soluble film or pouch.

Exemplary laundry detergent compositions include, but are not limited to, for example, liquid and powder laundry detergent compositions. Exemplary hard surface cleaning compositions include, but not limited to, for example, compositions used to clean the hard surface of a non-dishware item, non-tableware item, table, table top, furniture item, wall, floor, and ceiling. Exemplary hard surface cleaning compositions are described, for example, in U.S. Pat. Nos. 6,610,642, 6,376,450, and 6,376,450. Exemplary personal care compositions include, but are not limited to, compositions used to clean dentures, teeth, hair, contact lenses, and skin. Exemplary components of such oral care composition include those described in, for example, U.S. Pat. No. 6,376,450.

In some embodiments, one or more subtilisin variant described herein cleans at low temperatures. In other embodiments, one or more composition described herein cleans at low temperatures. In other embodiments, one or more composition described herein comprises an effective amount of one or more subtilisin variant described herein as useful or effective for cleaning a surface in need of proteinaceous stain removal.

In some embodiments, adjunct materials are incorporated, for example, to assist or enhance cleaning performance; for treatment of the substrate to be cleaned; or to modify the aesthetics of the cleaning composition as is the case with perfumes, colorants, dyes or the like. One embodiment is directed to a composition comprising one or more adjunct material and one or more subtilisin variant described herein. Another embodiment is directed to a composition comprising one or more adjunct material and one or more subtilisin variant described herein, wherein the adjunct material is selected from a bleach catalyst, an additional enzyme, an enzyme stabilizer (including, for example, an enzyme stabilizing system), a chelant, an optical brightener, a soil release polymer, biodegradable polymer, a dye transfer agent, a dispersant, a suds suppressor, a dye, a perfume, a colorant, a filler, a photoactivator, a fluorescer, a fabric conditioner, a hydrolyzable surfactant, a preservative, an anti-oxidant, an anti-shrinkage agent, an anti-wrinkle agent, a germicide, a fungicide, a color speckle, a silvercare agent, an anti-tarnish agent, an anti-corrosion agent, an alkalinity source, a solubilizing agent, a carrier, a processing aid, a pigment, a pH control agent, a surfactant, a builder, a chelating agent, a dye transfer inhibiting agent, a deposition aid, a catalytic material, a bleach activator, a bleach booster, a hydrogen peroxide, a source of hydrogen peroxide, a preformed peracid, a polymeric dispersing agent, a clay soil removal/anti-redeposition agent, a structure elasticizing agent, a fabric softener, a carrier, a hydrotrope, a processing aid, a pigment, and combinations thereof. Exemplary adjunct materials and levels of use are found in U.S. Pat. Nos. 5,576,282; 6,306,812; 6,326,348; 6,610,642; 6,605,458; 5,705,464; 5,710,115; 5,698,504; 5,695,679; 5,686,014 and 5,646,101. In embodiments in which one or more cleaning adjunct material is not compatible with one or more subtilisin variant described herein, methods are employed to keep the adjunct material and variant(s) separated (i.e., not in contact with each other) until combination of the two components is appropriate. Such separation methods include any suitable method known in the art (e.g., gelcaps, encapsulation, tablets, physical separation, etc.).

Some embodiments are directed to cleaning additive products comprising one or more subtilisin variant described herein. In some embodiments, the additive is packaged in a dosage form for addition to a cleaning process. In some embodiments, the additive is packaged in a dosage form for addition to a cleaning process where a source of peroxide is employed and increased bleaching effectiveness is desired.

Exemplary fillers or carriers for granular compositions include, but are not limited to, for example, various salts of sulfate, carbonate and silicate; talc; and clay. Exemplary fillers or carriers for liquid compositions include, but are not limited to, for example, water or low molecular weight primary and secondary alcohols including polyols and diols (e.g., methanol, ethanol, propanol and isopropanol). In some embodiments, the compositions contain from about 5% to about 90% of such filler or carrier. Acidic fillers may be included in such compositions to reduce the pH of the resulting solution in the cleaning method or application.

In one embodiment, one or more cleaning composition described herein comprises an effective amount of one or more subtilisin variant described herein, alone or in combination with one or more additional enzyme. Typically, a cleaning composition comprises at least about 0.0001 to about 20 wt %, from about 0.0001 to about 10 wt %, from about 0.0001 to about 1 wt %, from about 0.001 to about 1 wt %, or from about 0.01 to about 0.2 wt % of one or more subtilisin variant described herein. In another embodiment, one or more cleaning composition described herein comprises from about 0.01 to about 10 mg, about 0.01 to about 5 mg, about 0.01 to about 2 mg, about 0.01 to about 1 mg, about 0.05 to about 1 mg, about 0.5 to about 10 mg, about 0.5 to about 5 mg, about 0.5 to about 4 mg, about 0.5 to about 3 mg, about 0.5 to about 2 mg, about 0.5 to about 1 mg, about 0.1 to about 10 mg, about 0.1 to about 5 mg, about 0.1 to about 4 mg, about 0.1 to about 3 mg, about 0.1 to about 2 mg, about 0.1 to about 2 mg, about 0.1 to about 1 mg, or about 0.1 to about 0.5 mg of one or more subtilisin variant described herein per gram of composition.

The cleaning compositions described herein are typically formulated such that during use in aqueous cleaning operations, the wash water will have a pH of from about 4.0 to about 11.5, or even from about 5.0 to about 11.5, or even from about 5.0 to about 8.0, or even from about 7.5 to about 10.5. Liquid product formulations are typically formulated to have a pH from about 3.0 to about 9.0 or even from about 3 to about 5. Granular laundry products are typically formulated to have a pH from about 8 to about 11. In some embodiments, the cleaning compositions of the present invention can be formulated to have an alkaline pH under wash conditions, such as a pH of from about 8.0 to about 12.0, or from about 8.5 to about 11.0, or from about 9.0 to about 11.0. In some embodiments, the cleaning compositions of the present invention can be formulated to have a neutral pH under wash conditions, such as a pH of from about 5.0 to about 8.0, or from about 5.5 to about 8.0, or from about 6.0 to about 8.0, or from about 6.0 to about 7.5. In some embodiments, the neutral pH conditions can be measured when the cleaning composition is dissolved 1:100 (wt:wt) in de-ionised water at 20° C., measured using a conventional pH meter. Techniques for controlling pH at recommended usage levels include the use of buffers, alkalis, acids, etc., and are well known to those skilled in the art.

In some embodiments, one or more subtilisin variant described herein is encapsulated to protect it during storage from the other components in the composition and/or control the availability of the variant during cleaning. In some embodiments, encapsulation enhances the performance of the variant and/or additional enzyme. In some embodiments, the encapsulating material typically encapsulates at least part of the subtilisin variant described herein. Typically, the encapsulating material is water-soluble and/or water-dispersible. In some embodiments, the encapsulating material has a glass transition temperature (Tg) of 0° C. or higher. Exemplary encapsulating materials include, but are not limited to, carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, and combinations thereof. In some embodiments, the encapsulating material is a starch (See e.g., EP0922499, U.S. Pat. Nos. 4,977,252, 5,354,559, and 5,935,826). In some embodiments, the encapsulating material is a microsphere made from plastic such as thermoplastics, acrylonitrile, methacrylonitrile, polyacrylonitrile, polymethacrylonitrile and mixtures thereof. Exemplary commercial microspheres include, but are not limited to EXPANCEL® (Stockviksverken, Sweden); and PM 6545, PM 6550, PM 7220, PM 7228, EXTENDOSPHERES®, LUXSIL®, Q-CEL®, and SPHERICEL® (PQ Corp., Valley Forge, PA).

There are a variety of wash conditions including varying detergent formulations, wash water volumes, wash water temperatures, and lengths of wash time to which one or more subtilisin variant described herein may be exposed. A low detergent concentration system is directed to wash water containing less than about 800 ppm detergent components. A medium detergent concentration system is directed to wash containing between about 800 ppm and about 2000 ppm detergent components. A high detergent concentration system is directed to wash water containing greater than about 2000 ppm detergent components. In some embodiments, the “cold water washing” of the present invention utilizes “cold water detergent” suitable for washing at temperatures from about 10° C. to about 40° C., from about 20° C. to about 30° C., or from about 15° C. to about 25° C., as well as all other combinations within the range of about 15° C. to about 35° C. or 10° C. to 40° C.

Different geographies have different water hardness. Hardness is a measure of the amount of calcium (Ca²⁺) and magnesium (Mg²⁺) in the water. Water hardness is usually described in terms of the grains per gallon (gpg) mixed Ca²⁺/Mg²⁺. Most water in the United States is hard, but the degree of hardness varies. Moderately hard (60-120 ppm) to hard (121-181 ppm) water has 60 to 181 ppm (ppm can be converted to grains per U.S. gallon by dividing ppm by 17.1) of hardness minerals.

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

Other embodiments are directed to one or more cleaning composition comprising from about 0.00001% to about 10% by weight composition of one or more subtilisin variant described herein and from about 99.999% to about 90.0% by weight composition of one or more adjunct material. In another embodiment, the cleaning composition comprises from about 0.0001% to about 10%, about 0.001% to about 5%, about 0.001% to about 2%, or about 0.005% to about 0.5% by weight composition of one or more subtilisin variant and from about 99.9999% to about 90.0%, about 99.999% to about 98%, about 99.995% to about 99.5% by weight composition of one or more adjunct material.

In other embodiments, the composition described herein comprises one or more subtilisin variant described herein and one or more additional enzyme. The one or more additional enzyme is selected from acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, dispersins, endo-beta-1,4-glucanases, endo-beta-mannanases, esterases, exo-mannanases, galactanases, glucoamylases, hemicellulases, hexosaminidase, hyaluronidases, keratinases, laccases, lactases, ligninases, lipases, lipoxygenases, lysozymes, mannanases, metalloproteases, nucleases (e.g. DNases and/or RNases), oxidases, oxidoreductases, pectate lyases, pectin acetyl esterases, pectinases, pentosanases, perhydrolases, peroxidases, phenoloxidases, phosphatases, phosphodiesterases, phospholipases, phytases, polygalacturonases, polyesterases, additional proteases, pullulanases, reductases, rhamnogalacturonases, beta-glucanases, tannases, transglutaminases, xanthan lyases, xylan acetyl-esterases, xylanases, xyloglucanases, xylosidases, and any combination or mixture thereof. Some embodiments are directed to a combination of enzymes (i.e., a “cocktail”) comprising conventional enzymes like amylase, lipase, cutinase, mannanase and/or cellulase in conjunction with one or more subtilisin variant described herein and/or one or more additional protease.

In another embodiment, one or more composition described herein comprises one or more subtilisin variant described herein and one or more additional protease. In one embodiment, the additional protease is a serine protease. In another embodiment, the additional protease is not immunologically related (e.g. the proteases do not cross-react in antibody-based tests known in the art to evaluate shared immunological epitopes). In another embodiment, the additional protease is a protease with a different net charge at pH 8. In another embodiment, the additional protease is a metalloprotease, a fungal subtilisin, or an alkaline microbial protease or a trypsin-like protease. Suitable additional proteases include those of animal, vegetable or microbial origin. In some embodiments, the additional protease is a microbial protease. In other embodiments, the additional protease is a chemically or genetically modified mutant. In another embodiment, the additional protease is an alkaline microbial protease or a trypsin-like protease. In other embodiments, the additional protease does not contain cross-reactive epitopes with the subtilisin variant as measured by antibody binding or other assays available in the art. Exemplary alkaline proteases include subtilisins derived from, for example, Bacillus (e.g., BPN′, Carlsberg, subtilisin 309, subtilisin 147, subtilisin PB92, subtilisin DY, subtilisin 168, B. gibsonii DSM14391 subtilisin, B. gibsonii AGS78407 subtilisin, B. pumilus DSM18097 subtilisin, B lentus DSM5483, Bacillus amyloliquefaciens, Bacillus xiamenensis, and B. sp TY-145 subtilisin), or fungal origin, such as, for example, those described in U.S. Pat. No. 8,362,222. Exemplary additional proteases include but are not limited to those described in WO92/21760, WO95/23221, WO2008/010925, WO09/149200, WO09/149144, WO09/149145, WO 10/056640, WO10/056653, WO2010/0566356, WO11/072099, WO2011/13022, WO11/140364, WO 12/151534, WO2015/038792, WO2015/089447, WO2015/089441, WO 2017/215925, US Publ. No. 2008/0090747, U.S. Pat. Nos. 5,801,039, 5,340,735, 5,500,364, 5,855,625, RE 34,606, U.S. Pat. Nos. 5,955,340, 5,700,676 6,312,936, 6,482,628, 8,530,219, U.S. Provisional Appl Nos. 62/180,673 and 62/161,077, and PCT Appl Nos. PCT/US2015/021813, PCT/US2015/055900, PCT/US2015/057497, PCT/US2015/057492, PCT/US2015/057512, PCT/US2015/057526, PCT/US2015/057520, PCT/US2015/057502, PCT/US2016/022282, WO2016074925, WO2020178102, WO2022106400, WO2016203064, EP3380599, WO2017215925, WO201948495, WO2020221578, WO2016203064, U.S. Pat. No. 7,294,499, WO2016/097354, WO2020112599, WO2021175696, WO2021175697, and PCT/US16/32514, as well as metalloproteases described in WO1999014341, WO1999033960, WO1999014342, WO1999034003, WO2007044993, WO2009058303, WO 2009058661, WO2014071410, WO2014194032, WO2014194034, WO2014194054, and WO2014/194117.

Exemplary additional proteases include, but are not limited to trypsin (e.g., of porcine or bovine origin) and the Fusarium protease described in WO89/06270. Exemplary commercial proteases include, but are not limited to MAXATASE®, MAXACAL™, MAXAPEM™, OPTICLEAN®, OPTIMASE®, PROPERASE®, PURAFECT®, PURAFECT® OXP, PURAMAX™, EXCELLASE™, PREFERENZ™ (e.g. P100, P110, P280, P300), EFFECTENZ™ (e.g. P1000, P1050, P2000), EXCELLENZ™ (e.g. P1000), ULTIMASE®, and PURAFAST™ proteases (Danisco/Genencor/IFF); ALCALASE®, ALCALASE®, ULTRA, BLAZER, BLAZE® variants, BLAZE® EVITY®, BLAZER EVITY® 16L, CORONASE®, SAVINASE®, SAVINASE® ULTRA, SAVINASE® EVITY®, SAVINASE® EVERIS®, PRIMASE®, DURAZYM™, POLARZYME®, OVOZYME®, KANNASE®, LIQUANASE®, LIQUANASE EVERIS®, NEUTRASE®, PROGRESS UNO®, RELASE®, and ESPERASER (Novozymes); BLAP™ and BLAP™ variants (Henkel); LAVERGY™ PRO 104 L (BASF). KAP (B. alkalophilus subtilisin (Kao)) and BIOTOUCH® (AB Enzymes).

Another embodiment is directed to a composition comprising one or more subtilisin variant described herein and one or more lipase. In some embodiments, the composition comprises from about 0.00001% to about 10%, about 0.0001% to about 10%, about 0.001% to about 5%, about 0.001% to about 2%, or about 0.005% to about 0.5% lipase by weight composition. An exemplary lipase can be a chemically or genetically modified mutant. Exemplary lipases include, but are not limited to, e.g., those of bacterial or fungal origin, such as, e.g., H. lanuginosa lipase (see, e.g., EP 258068 and EP 305216), T. lanuginosa lipase (see, e.g., WO 2014/059360 and WO2015/010009), Rhizomucor miehei lipase (see, e.g., EP 238023), Candida lipase, such as C. antarctica lipase (e.g., C. antarctica lipase A or B) (see, e.g., EP 214761), Pseudomonas lipases such as P. alcaligenes and P. pseudoalcaligenes lipase (see, e.g., EP 218272), P. cepacia lipase (see, e.g., EP 331376), P. stutzeri lipase (see, e.g., GB 1,372,034), P. fluorescens lipase, Bacillus lipase (e.g., B. subtilis lipase (Dartois et al., Biochem. Biophys. Acta 1131:253-260 (1993)), B. stearothermophilus lipase (see, e.g., JP 64/744992), and B. pumilus lipase (see, e.g., WO 91/16422)). Exemplary cloned lipases include, but are not limited to Penicillium camembertii lipase (See, Yamaguchi et al., Gene 103:61-67 (1991)), Geotricum candidum lipase (See, Schimada et al., J. Biochem., 106:383-388 (1989)), and various Rhizopus lipases, such as, R. delemar lipase (See, Hass et al., Gene 109:117-113 (1991)), R. niveus lipase (Kugimiya et al., Biosci. Biotech. Biochem. 56:716-719 (1992)) and R. oryzae lipase. Other lipolytic enzymes, such as cutinases, may also find use in one or more composition described herein, including, but not limited to, e.g., cutinase derived from Pseudomonas mendocina (see, WO 88/09367) and/or Fusarium solani pisi (see, WO90/09446). Exemplary commercial lipases include, but are not limited to MI LIPASE™, LUMA FAST™, LIPOMAX™, and PREFERENZ™ L100 (IFF); LIPEX®, LIPOCLEAN®, LIPOLASE® and LIPOLASE® ULTRA (Novozymes); and LIPASE P™ (Amano Pharmaceutical Co. Ltd).

A still further embodiment is directed to a composition comprising one or more subtilisin variant described herein and one or more amylase. In one embodiment, the composition comprises from about 0.00001% to about 10%, about 0.0001% to about 10%, about 0.001% to about 5%, about 0.001% to about 2%, or about 0.005% to about 0.5% amylase by weight composition. Any amylase (e.g., alpha and/or beta) suitable for use in alkaline solutions may be useful to include in such composition. An exemplary amylase can be a chemically or genetically modified mutant. Exemplary amylases include, but are not limited to those of bacterial or fungal origin, such as, for example, amylases described in GB 1,296,839, WO9100353, WO9402597, WO94183314, WO9510603, WO9526397, WO9535382, WO9605295, WO9623873, WO9623874, WO 9630481, WO9710342, WO9741213, WO9743424, WO9813481, WO 9826078, WO9902702, WO 9909183, WO9919467, WO9923211, WO9929876, WO9942567, WO 9943793, WO9943794, WO 9946399, WO0029560, WO0060058, WO0060059, WO0060060, WO 0114532, WO0134784, WO 0164852, WO0166712, WO0188107, WO0196537, WO02092797, WO 0210355, WO0231124, WO 2004055178, WO2004113551, WO2005001064, WO2005003311, WO 2005018336, WO2005019443, WO2005066338, WO2006002643, WO2006012899, WO2006012902, WO2006031554, WO 2006063594, WO2006066594, WO2006066596, WO2006136161, WO 2008000825, WO2008088493, WO2008092919, WO2008101894, WO2008/112459, WO2009061380, WO2009061381, WO 2009100102, WO2009140504, WO2009149419, WO 2010/059413, WO 2010088447, WO2010091221, WO2010104675, WO2010115021, WO10115028, WO2010117511, WO 2011076123, WO2011076897, WO2011080352, WO2011080353, WO 2011080354, WO2011082425, WO2011082429, WO 2011087836, WO2011098531, WO2013063460, WO2013184577, WO 2014099523, WO2014164777, WO2015077126, WO2022/175435, and WO2018184004. Exemplary commercial amylases include, but are not limited to AMPLIFY®, DURAMYL®, TERMAMYL®, FUNGAMYL®), STAINZYME®, STAINZYME PLUS®, STAINZYME PLUS®, AMPLIFY PRIME®, STAINZYME ULTRA® EVITY®, and BAN™ (Novozymes); EFFECTENZ™ S 1000, POWERASE™, PREFERENZ™ S 100, PREFERENZ™ S 110, PREFERENZ™ S 210, EXCELLENZ™ S 2000, RAPIDASE® and MAXAMYL® P (IFF). In some embodiments, the subtilisin variants provided herein may be combined with one or more amylases selected from the group consisting of AA707, AA560, AA110, BspAmy24, SP722, and CspAmyl, and variants thereof, and combinations thereof.

Yet a still further embodiment is directed to a composition comprising one or more subtilisin variant described herein and one or more cellulase. In one embodiment, the composition comprises from about 0.00001% to about 10%, 0.0001% to about 10%, about 0.001% to about 5%, about 0.001% to about 2%, or about 0.005% to about 0.5% cellulase by weight of composition. Any suitable cellulase may find use in a composition described herein. An exemplary cellulase can be a chemically or genetically modified mutant. Exemplary cellulases include but are not limited, to those of bacterial or fungal origin, such as, for example, those described in WO2005054475, WO2005056787, U.S. Pat. Nos. 7,449,318, 7,833,773, 4,435,307; EP 0495257; and U.S. Provisional Appl. No. 62/296,678. Exemplary commercial cellulases include, but are not limited to, CELLUCLEAN®, CELLUZYME®, CAREZYME®, ENDOLASE®, RENOZYME®, and CAREZYME® PREMIUM (Novozymes); REVITALENZ™ 100, REVITALENZ™ 200/220, and REVITALENZ® 2000 (IFF); and KAC-500 (B)™ (Kao Corporation). In some embodiments, cellulases are incorporated as portions or fragments of mature wild-type or variant cellulases, wherein a portion of the N-terminus is deleted (see, e.g., U.S. Pat. No. 5,874,276).

An even still further embodiment is directed to a composition comprising one or more subtilisin variant described herein and one or more mannanase. In one embodiment, the composition comprises from about 0.00001% to about 10%, about 0.0001% to about 10%, about 0.001% to about 5%, about 0.001% to about 2%, or about 0.005% to about 0.5% mannanase by weight composition. An exemplary mannanase can be a chemically or genetically modified mutant. Exemplary mannanases include, but are not limited to, those of bacterial or fungal origin, such as, for example, those described in WO 2016/007929; U.S. Pat. Nos. 6,566,114; 6,602,842; and 6,440,991; and U.S. Provisional Appl. Nos. 62/251,516, 62/278,383, and 62/278387. Exemplary commercial mannanases include, but are not limited to MANNAWAY® (Novozymes) and EFFECTENZ™ M 1000, EFFECTENZ™ M 2000, PREFERENZ® M 100, MANNASTAR®, and PURABRITE™ (IFF), and BIOTOUCH® (AB Enzymes),

A still further embodiment is directed to a composition comprising one or more subtilisin variant described herein and one or more nuclease, such as a DNase or RNase. In one embodiment, the composition comprises from about 0.00001% to about 10%, about 0.0001% to about 10%, about 0.001% to about 5%, about 0.001% to about 2%, or about 0.005% to about 0.5% nuclease by weight composition. Exemplary nucleases include, but are not limited to, those described in WO2015181287, WO2015155350, WO2016162556, WO2017162836, WO2017060475 (e.g. SEQ ID NO: 21), WO2018184816, WO2018177936, WO2018177938, WO2018/185269, WO2018185285, WO2018177203, WO2018184817, WO2019084349, WO2019084350, WO2019081721, WO2018076800, WO2018185267, WO2018185280, WO2018206553 and WO2020099490. Other nucleases which can be used in combination with the substilisin variants provided herein in the compositions and methods provided herein include those described in Nijland R, Hall M J, Burgess J G (2010) Dispersal of Biofilms by Secreted, Matrix Degrading, Bacterial DNase. PLOS ONE 5 (12) and Whitchurch, C. B., Tolker-Nielsen, T., Ragas, P. C., Mattick, J. S. (2002) Extracellular DNA required for bacterial biofilm formation. Science 295:1487.

A yet even still further embodiment is directed to a composition comprising one or more subtilisin variant described herein and one or more peroxidase and/or oxidase enzyme. In one embodiment, the composition comprises from about 0.00001% to about 10%, about 0.0001% to about 10%, about 0.001% to about 5%, about 0.001% to about 2%, or about 0.005% to about 0.5% peroxidase or oxidase by weight composition. A peroxidase may be used in combination with hydrogen peroxide or a source thereof (e.g., a percarbonate, perborate or persulfate) and an oxidase may be used in combination with oxygen. Peroxidases and oxidases are used for “solution bleaching” (i.e., to prevent transfer of a textile dye from a dyed fabric to another fabric when the fabrics are washed together in a wash liquor), alone or in combination with an enhancing agent (see, e.g., WO94/12621 and WO95/01426). An exemplary peroxidase and/or oxidase can be a chemically or genetically modified mutant. Exemplary peroxidases/oxidases include, but are not limited to those of plant, bacterial, or fungal origin.

Another embodiment is directed to a composition comprising one or more subtilisin variant described herein, and one or more perhydrolase, such as, for example, is described in WO2005/056782, WO2007/106293, WO 2008/063400, WO2008/106214, and WO2008/106215.

Another embodiment is directed to a composition comprising one or more subtilisin variant described herein, and an engineered polysaccharide biopolymer with cleaning/whitening improvement or textile surface modification benefits.

In yet another embodiment, the one or more subtilisin variant described herein and one or more additional enzyme contained in one or more composition described herein may each independently range to about 10% by weight composition, wherein the balance of the cleaning composition is one or more adjunct material.

In some embodiments, one or more composition described herein finds use as a detergent additive, wherein said additive is in a solid or liquid form. Such additive products are intended to supplement and/or boost the performance of conventional detergent compositions and can be added at any stage of the cleaning process. In some embodiments, the density of the laundry detergent composition ranges from about 400 to about 1200 g/liter, while in other embodiments it ranges from about 500 to about 950 g/liter of composition measured at 20° C.

Some embodiments are directed to a laundry detergent composition comprising one or more subtilisin variant described herein and one or more adjunct material selected from surfactants, enzyme stabilizers, builder compounds, polymeric compounds, bleaching agents, additional enzymes, suds suppressors, dispersants, lime-soap dispersants, soil suspension agents, anti-redeposition agents, corrosion inhibitors, and combinations thereof. In some embodiments, the laundry compositions also contain softening agents.

Further embodiments are directed to manual dishwashing composition comprising one or more subtilisin variant described herein and one or more adjunct material selected from surfactants, organic polymeric compounds, suds enhancing agents, group II metal ions, solvents, hydrotropes, and additional enzymes.

Other embodiments are directed to one or more composition described herein, wherein said composition is a compact granular fabric cleaning composition that finds use in laundering colored fabrics or provides softening through the wash capacity, or is a heavy duty liquid (HDL) fabric cleaning composition. Exemplary fabric cleaning compositions and/or processes for making are described in U.S. Pat. Nos. 6,610,642 and 6,376,450. Other exemplary cleaning compositions are described, for example, in U.S. Pat. Nos. 6,605,458; 6,294,514; 5,929,022; 5,879,584; 5,691,297; 5,565,145; 5,574,005; 5,569,645; 5,565,422; 5,516,448; 5,489,392; and U.S. Pat. Nos. 5,486,303; 4,968,451; 4,597,898; 4,561,998; 4,550,862; 4,537,706; 4,515,707; and 4,515,705.

In some embodiments, the cleaning compositions comprise an acidifying particle or an amino carboxylic builder. Examples of an amino carboxylic builder include aminocarboxylic acids, salts and derivatives thereof. In some embodiment, the amino carboxylic builder is an aminopolycarboxylic builder, such as glycine-N,N-diacetic acid or derivative of general formula MOOC—CHR—N(CH₂COOM)₂ where R is C₁₋₁₂alkyl and M is alkali metal. In some embodiments, the amino carboxylic builder can be methylglycine diacetic acid (MGDA), GLDA (glutamic-N,N-diacetic acid), iminodisuccinic acid (IDS), carboxymethyl inulin and salts and derivatives thereof, aspartic acid-N-monoacetic acid (ASMA), aspartic acid-N,N-diacetic acid (ASDA), aspartic acid-N-monopropionic acid (ASMP), iminodiacetic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), IDA (iminodiacetic acid) and salts and derivatives thereof such as N-methyliminodiacetic acid (MIDA), alpha-alanine-N,N-diacetic acid (alpha-ALDA), serine-N,N-diacetic acid (SEDA), isoserine-N,Ndiacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts and derivative thereof. In some embodiments, the acidifying particle has a weight geometric mean particle size of from about 400u to about 1200u and a bulk density of at least 550 g/L. In some embodiments, the acidifying particle comprises at least about 5% of the builder.

In some embodiments, the acidifying particle can comprise any acid, including organic acids and mineral acids. Organic acids can have one or two carboxyls and in some instances up to 15 carbons, especially up to 10 carbons, such as formic, acetic, propionic, capric, oxalic, succinic, adipic, maleic, fumaric, sebacic, malic, lactic, glycolic, tartaric and glyoxylic acids. In some embodiments, the acid is citric acid. Mineral acids include hydrochloric and sulphuric acid. In some instances, the acidifying particle is a highly active particle comprising a high level of amino carboxylic builder. Sulphuric acid has also been found to further contribute to the stability of the final particle.

Additional embodiments are directed to a cleaning composition comprising one or more subtilisin variant and one or more surfactant and/or surfactant system, wherein the surfactant is selected from nonionic surfactants, anionic surfactants, cationic surfactants, ampholytic surfactants, zwitterionic surfactants, semi-polar nonionic surfactants, and mixtures thereof. In some embodiments, the surfactant is present at a level of from about 0.1 to about 60%, while in alternative embodiments the level is from about 1 to about 50%, while in still further embodiments the level is from about 5 to about 40%, by weight of the cleaning composition.

In some embodiments, one or more composition described herein comprises one or more detergent builders or builder systems. In one embodiment, the composition comprises from at least about 0.1% or greater, or from about 0.1% to about 90%, from about 0.1% to about 80%, from about 3% to about 60%, from about 5% to about 40%, or from about 10% to about 50% builder by weight composition. Exemplary builders include, but are not limited to alkali metal; ammonium and alkanolammonium salts of polyphosphates; alkali metal silicates; alkaline earth and alkali metal carbonates; aluminosilicates; polycarboxylate compounds; ether hydroxypolycarboxylates; copolymers of maleic anhydride with ethylene or vinyl methyl ether, 1, 3, 5-trihydroxy benzene-2, 4, 6-trisulphonic acid, and carboxymethyloxysuccinic acid; ammonium and substituted ammonium salts of polyacetic acids such as ethylenediamine tetraacetic acid and nitrilotriacetic acid; polycarboxylates such as mellitic acid, succinic acid, citric acid, oxydisuccinic acid, polymaleic acid, benzene 1,3,5-tricarboxylic acid, carboxymethyloxysuccinic acid; and soluble salts thereof. In some such compositions, the builders form water-soluble hardness ion complexes (e.g., sequestering builders), such as citrates and polyphosphates, e.g., sodium tripolyphosphate, sodium tripolyphospate hexahydrate, potassium tripolyphosphate, and mixed sodium and potassium tripolyphosphate. Exemplary builders are described in, e.g., EP 2100949. In some embodiments, the builders include phosphate builders and non-phosphate builders. In some embodiments, the builder is a phosphate builder. In some embodiments, the builder is a non-phosphate builder. In some embodiments, the builder comprises a mixture of phosphate and non-phosphate builders. Exemplary phosphate builders include, but are not limited to mono-phosphates, di-phosphates, tri-polyphosphates or oligomeric-polyphosphates, including the alkali metal salts of these compounds, including the sodium salts. In some embodiments, a builder can be sodium tripolyphosphate (STPP). Additionally, the composition can comprise carbonate and/or citrate. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above-mentioned compounds include the ammonium and/or alkali metal salts, i.e. the lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

In some embodiments, one or more composition described herein comprises one or more chelating agent. In one embodiment, the composition comprises from about 0.1% to about 15% or about 3% to about 10% chelating agent by weight composition. Exemplary chelating agents include, but are not limited to, e.g., copper, iron, manganese, and mixtures thereof.

In some embodiments, one or more composition described herein comprises one or more deposition aid. Exemplary deposition aids include, but are not limited to, e.g., polyethylene glycol; polypropylene glycol; polycarboxylate; soil release polymers, such as, e.g., polyterephthalic acid; clays such as, e.g., kaolinite, montmorillonite, attapulgite, illite, bentonite, and halloysite; and mixtures thereof.

In other embodiments, one or more composition described herein comprises one or more anti-redeposition agent or non-ionic surfactant (which can prevent the re-deposition of soils) (see, e.g., EP 2100949). For example, in ADW compositions, non-ionic surfactants find use for surface modification purposes, in particular for sheeting, to avoid filming and spotting and to improve shine. These non-ionic surfactants also find use in preventing the re-deposition of soils. In some embodiments, the non-ionic surfactant can be ethoxylated nonionic surfactants, epoxy-capped poly(oxyalkylated) alcohols and amine oxides surfactants.

In some embodiments, one or more composition described herein comprises one or more dye transfer inhibiting agent. Exemplary polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, polyvinylimidazoles, and mixtures thereof. In one embodiment, the composition comprises from about 0.0001% to about 10%, about 0.01% to about 5%, or about 0.1% to about 3% dye transfer inhibiting agent by weight composition.

In some embodiments, one or more composition described herein comprises one or more silicate. Exemplary silicates include, but are not limited to, sodium silicates, e.g., sodium disilicate, sodium metasilicate, and crystalline phyllosilicates. In some embodiments, silicates are present at a level of from about 1% to about 20% or about 5% to about 15% by weight of the composition.

In some still additional embodiments, one or more composition described herein comprises one or more dispersant. Exemplary water-soluble organic materials include, but are not limited to, e.g., homo- or co-polymeric acids or their salts, in which the polycarboxylic acid comprises at least two carboxyl radicals separated from each other by not more than two carbon atoms.

In some further embodiments, one or more composition described herein comprises one or more enzyme stabilizer. In some embodiments, the enzyme stabilizer is water-soluble sources of calcium and/or magnesium ions. In some embodiments, the enzyme stabilizers include oligosaccharides, polysaccharides, and inorganic divalent metal salts, including alkaline earth metals, such as calcium salts. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV)). Chlorides and sulfates also find use in some embodiments. Exemplary oligosaccharides and polysaccharides (e.g., dextrins) are described, for example, in WO 07/145964. In some embodiments, reversible protease inhibitors also find use, such as boron-containing compounds (e.g., borate, 4-formyl phenyl boronic acid, and phenyl-boronic acid derivatives (such as for example, those described in WO96/41859)) and/or a peptide aldehyde, such as, for example, is further described in WO2009/118375 and WO2013004636.

Peptide aldehydes may be used as protease stabilizers in detergent formulations as previously described (WO199813458, WO2011036153, US20140228274). Examples of peptide aldehyde stabilizers are peptide aldehydes, ketones, or halomethyl ketones and might be ‘N-capped’ with for instance a ureido, a carbamate, or a urea moiety, or ‘doubly N-capped’ with for instance a carbonyl, a ureido, an oxiamide, a thioureido, a dithiooxamide, or a thiooxamide moiety (EP2358857B1). The molar ratio of these inhibitors to the protease may be 0.1:1 to 100:1, e.g. 0.5:1-50:1, 1:1-25:1 or 2:1-10:1. Other examples of protease stabilizers are benzophenone or benzoic acid anilide derivatives, which might contain carboxyl groups (U.S. Pat. No. 7,968,508 B2). The molar ratio of these stabilizers to protease is preferably in the range of 1:1 to 1000:1 in particular 1:1 to 500:1 especially preferably from 1:1 to 100:1, most especially preferably from 1:1 to 20:1.

In some embodiments, one or more composition described herein comprises one or more bleach, bleach activator, and/or bleach catalyst. In some embodiments, one or more composition described herein comprises one or more inorganic and/or organic bleaching compound. Exemplary inorganic bleaches include, but are not limited to perhydrate salts, e.g., perborate, percarbonate, perphosphate, persulfate, and persilicate salts. In some embodiments, inorganic perhydrate salts are alkali metal salts. In some embodiments, inorganic perhydrate salts are included as the crystalline solid, without additional protection, although in some other embodiments, the salt is coated. Bleach activators are typically organic peracid precursors that enhance the bleaching action in the course of cleaning at temperatures of 60° C. and below. Exemplary bleach activators include compounds which, under perhydrolysis conditions, give aliphatic peroxycarboxylic acids having from about 1 to about 10 carbon atoms or about 2 to about 4 carbon atoms, and/or optionally substituted perbenzoic acid. Exemplary bleach activators as described, for example, in EP 2100949. Exemplary bleach catalysts include, but are not limited to, manganese triazacyclononane and related complexes, as well as cobalt, copper, manganese, and iron complexes. Additional exemplary bleach catalysts are described, for example, in U.S. Pat. Nos. 4,246,612; 5,227,084; 4,810,410; WO 99/06521; and EP 2100949.

In some embodiments, one or more composition described herein comprises one or more catalytic metal complexes. In some embodiments, a metal-containing bleach catalyst finds use. In some embodiments, the metal bleach catalyst comprises a catalyst system comprising a transition metal cation of defined bleach catalytic activity (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof (see, e.g., U.S. Pat. No. 4,430,243). In some embodiments, one or more composition described herein is catalyzed by means of a manganese compound. Such compounds and levels of use are described, for example, in U.S. Pat. No. 5,576,282. In additional embodiments, cobalt bleach catalysts find use and are included in one or more composition described herein. Various cobalt bleach catalysts are described, for example, in U.S. Pat. Nos. 5,597,936 and 5,595,967.

In some additional embodiments, one or more composition described herein includes a transition metal complex of a macropolycyclic rigid ligand (MRL). As a practical matter, and not by way of limitation, in some embodiments, the compositions and cleaning processes described herein are adjusted to provide on the order of at least one part per hundred million, from about 0.005 ppm to about 25 ppm, about 0.05 ppm to about 10 ppm, or about 0.1 ppm to about 5 ppm of active MRL in the wash liquor. Exemplary MRLs include, but are not limited to special ultra-rigid ligands that are cross-bridged, such as, e.g., 5,12-diethyl-1,5,8,12-tetraazabicyclo (6.6.2) hexadecane. Exemplary metal MRLs are described, for example, in WO 2000/32601 and U.S. Pat. No. 6,225,464.

In another embodiment, one or more composition described herein comprises one or more metal care agent. In some embodiments, the composition comprises from about 0.1% to about 5% metal care agent by weight composition. Exemplary metal care agents include, for example, aluminum, stainless steel, and non-ferrous metals (e.g., silver and copper). Additional exemplary metal care agents are described, for example, in EP 2100949, WO 94/26860, and WO 94/26859. In some compositions, the metal care agent is a zinc salt.

In some embodiments, the cleaning composition is a heavy-duty liquid (HDL) composition comprising one or more subtilisin variant described herein. The HDL liquid laundry detergent can comprise a detersive surfactant (10-40%) comprising anionic detersive surfactant selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof; and optionally non-ionic surfactant selected from a group of linear or branched or random chain, substituted or unsubstituted alkyl alkoxylated alcohol, for example, a C₈-C₁₈alkyl ethoxylated alcohol and/or C₆-C₁₂alkyl phenol alkoxylates, optionally wherein the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from alkanolamine sulpho-betaines); ampholytic surfactants; semi-polar non-ionic surfactants; and mixtures thereof.

In another embodiment, the cleaning composition is a liquid or gel detergent, which is not unit dosed, that may be aqueous, typically containing at least 20% and up to 95% water by weight, such as up to about 70% water by weight, up to about 65% water by weight, up to about 55% water by weight, up to about 45% water by weight, or up to about 35% water by weight. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

The composition can comprise optionally, a surfactancy boosting polymer consisting of amphiphilic alkoxylated grease cleaning polymers selected from a group of alkoxylated polymers having branched hydrophilic and hydrophobic properties, such as alkoxylated polyalkylenimines in the range of 0.05 wt %-10 wt % and/or random graft polymers typically comprising a hydrophilic backbone comprising monomers selected from the group consisting of: unsaturated C₁-C₆carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C₄-C₂₅alkyl group, polypropylene, polybutylene, vinyl ester of a saturated C₂-C₆mono-carboxylic acid, C₁-C₆alkyl ester of acrylic or methacrylic acid, and mixtures thereof.

The composition can comprise additional polymers such as soil release polymers including, for example, anionically end-capped polyesters, for example SRP1; polymers comprising at least one monomer unit selected from saccharide, dicarboxylic acid, polyol and combinations thereof, in random or block configuration; ethylene terephthalate-based polymers and co-polymers thereof in random or block configuration, for example, Repel-o-tex SF, SF-2 and SRP6, Texcare SRA100, SRA300, SRN100, SRN170, SRN240, SRN300 and SRN325, Marloquest SL; anti-redeposition polymers (0.1 wt % to 10 wt %, including, for example, carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof; vinylpyrrolidone homopolymer; and/or polyethylene glycol with a molecular weight in the range of from 500 to 100,000 Da); cellulosic polymer (including, for example, alkyl cellulose; alkyl alkoxyalkyl cellulose; carboxyalkyl cellulose; alkyl carboxyalkyl cellulose, examples of which include carboxymethyl cellulose, methyl cellulose, methyl hydroxyethyl cellulose, methyl carboxymethyl cellulose; and mixtures thereof); and polymeric carboxylate (such as, for example, maleate/acrylate random copolymer or polyacrylate homopolymer).

The composition can further comprise saturated or unsaturated fatty acid, preferably saturated or unsaturated C₁₂-C₂₄fatty acid (0-10 wt %); deposition aids (including, for example, polysaccharides, cellulosic polymers, polydiallyl dimethyl ammonium halides (DADMAC), and co-polymers of DADMAC with vinyl pyrrolidone, acrylamides, imidazoles, imidazolinium halides, and mixtures thereof, in random or block configuration; cationic guar gum; cationic cellulose such as cationic hydroxyethyl cellulose; cationic starch; cationic polyacylamides; and mixtures thereof.

The composition can further comprise dye transfer inhibiting agents examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetracetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethylethylenediaminetri-acetic acid (HEDTA), triethylenetetraaminchexaacetic acid (TTHA), N-hydroxyethyliminodiacetic acid (HEIDA), dihydroxyethylglycine (DHEG), ethylenediaminetetrapropionic acid (EDTP), and derivatives thereof.

The composition can further comprise silicone or fatty-acid based suds suppressors; an enzyme stabilizer; hueing dyes, calcium and magnesium cations, visual signaling ingredients, anti-foam (0.001 to about 4.0 wt %), and/or structurant/thickener (0.01-5 wt %) selected from the group consisting of diglycerides, triglycerides, ethylene glycol distearate, microcrystalline cellulose, cellulose based materials, microfiber cellulose, biopolymers, xanthan gum, gellan gum, and mixtures thereof.

In some embodiments, the cleaning composition is a heavy duty powder (HDD) composition comprising one or more subtilisin variant described herein. The HDD powder laundry detergent can comprise a detersive surfactant including anionic detersive surfactants (selected from linear or branched or random chain, substituted or unsubstituted alkyl sulphates, alkyl sulphonates, alkyl alkoxylated sulphate, alkyl phosphates, alkyl phosphonates, alkyl carboxylates and/or mixtures thereof), non-ionic detersive surfactant (selected from linear or branched or random chain, substituted or unsubstituted C₈-C₁₈ alkyl ethoxylates, and/or C₆-C₁₂ alkyl phenol alkoxylates), cationic detersive surfactants (selected from alkyl pyridinium compounds, alkyl quaternary ammonium compounds, alkyl quaternary phosphonium compounds, alkyl ternary sulphonium compounds, and mixtures thereof); zwitterionic and/or amphoteric detersive surfactants (selected from alkanolamine sulpho-betaines); ampholytic surfactants; semi-polar non-ionic surfactants and mixtures thereof; builders (phosphate free builders, e,g., zeolite builders examples of which include zeolite A, zeolite X, zeolite P and zeolite MAP in the range of 0 to less than 10 wt %); phosphate builders, e.g., sodium tripolyphosphate in the range of 0 to less than 10 wt %; citric acid, citrate salts and nitrilotriacetic acid or salt thereof in the range of less than 15 wt %; silicate salt (sodium or potassium silicate or sodium meta-silicate in the range of 0 to less than 10 wt % or layered silicate (SKS-6)); carbonate salt (sodium carbonate and/or sodium bicarbonate in the range of 0 to less than 10 wt %); and bleaching agents (photobleaches, e.g., sulfonated zinc phthalocyanines, sulfonated aluminum phthalocyanines, xanthenes dyes, and mixtures thereof); hydrophobic or hydrophilic bleach activators (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, and nonanoyloxybenzene sulfonate-NOBS, nitrile quats, and mixtures thereof); hydrogen peroxide; sources of hydrogen peroxide (inorganic perhydrate salts, e.g., mono or tetra hydrate sodium salt of perborate, percarbonate, persulfate, perphosphate, or persilicate); preformed hydrophilic and/or hydrophobic peracids (selected from percarboxylic acids and salts, percarbonic acids and salts, perimidic acids and salts, peroxymonosulfuric acids and salts, and mixtures thereof); and/or bleach catalyst (e.g., imine bleach boosters, such as iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof), metal-containing bleach catalyst (e.g., copper, iron, titanium, ruthenium, tungsten, molybdenum, or manganese cations along with an auxiliary metal cations such as zinc or aluminum and a sequestrate such as ethylenediaminetetraacetic acid, ethylenediaminetetra (methylenephosphonic acid) and water-soluble salts thereof).

The composition can further comprise additional detergent ingredients including perfume microcapsules, starch encapsulated perfume accord, an enzyme stabilizer, hueing agents, additional polymers including fabric integrity and cationic polymers, dye lock ingredients, fabric-softening agents, brighteners (for example C.I. Fluorescent brighteners), flocculating agents, chelating agents, alkoxylated polyamines, fabric deposition aids, and/or cyclodextrin.

In some embodiments, the cleaning compositions comprising one or more subtilisin variant described herein is a detergent composition selected from the group consisting of a laundry detergent, a soap bar, a fabric softening detergent, a dishwashing detergent, a medical instrument detergent, and a hard-surface cleaning detergent.

In some embodiments, the invention is directed to detergent compositions comprising at least two proteases in combination with one or more additional cleaning composition components such as, but not limiting to, a liquid laundry composition described in WO2022106404.

In some embodiments, the one or more subtilisin variant described herein can be part of, or added to, a liquid laundry detergent composition such as, but not limiting to, a liquid laundry composition described in U.S. Pat. No. 11,046,919B2, WO2021/223552, WO2022/167251, WO2022/074037, WO2021/123184, WO2021/037895, WO2022/10372, WO2020/264077, WO2022/106404 and/or WO2017/54983; a compacted liquid laundry composition (U.S. Pat. No. 10,683,474B2); a water-soluble unit dose article comprising a fatty alkyl ester alkoxylate non-ionic surfactant and an alkoxylated alcohol non-ionic surfactant (US20220162523A1); a liquid laundry detergent composition comprising improved alkylbenzenesulfonate surfactants (WO2021/108307); a liquid laundry detergent composition comprising benzyl benzoate (WO2020/223959); and/or a detergent compositions containing a branched surfactant (WO2021/247801), a water-soluble unit dose article comprising an amphiphilic graft polymer and a polyester terephthalate (WO2019/032257).

In some embodiments, the cleaning compositions comprising one or more subtilisin variant described herein is a liquid laundry detergent composition containing alkyl ether carboxylic acids, betaines, anionic surfactant, non-ionic surfactant for providing softening benefits (WO2013/087286).

In some embodiments, the cleaning compositions comprising one or more subtilisin variant described herein is a liquid laundry detergent composition containing sulfite radical scavengers, protease stabilizers/inhibitors or combinations thereof (WO2022/157311).

In some embodiments, the cleaning composition comprising one or more subtilisin variant described herein is a liquid laundry detergent composition as described in US20210317387A1, WO2021/219296, WO2021/127662, WO2021/041685, U.S. Pat. No. 11,208,619, US20220186144.

In some embodiments, the cleaning composition comprising one or more subtilisin variant described herein is a liquid laundry detergent composition comprising dispersin variants, such as but limiting to a liquid laundry detergent composition described in US20210317387A1.

In some embodiments, the cleaning composition comprising one or more subtilisin variant described herein is a liquid laundry detergent composition is a highly alkaline textile washing agent, such as but limiting to a liquid laundry detergent composition described in WO2021/219296.

In some embodiments, the cleaning composition comprising one or more subtilisin variant described herein is a liquid laundry detergent composition is a low density unit dose detergent with encapsulated fragrance, such as but limiting to a detergent composition described in WO2021/127662.

In some embodiments, the cleaning composition comprising one or more subtilisin variant described herein is a liquid laundry detergent composition containing polyethylene glycol and an organic acid, such as but limiting to, a detergent composition described in WO2021/041685.

In some embodiments, the cleaning composition comprising one or more subtilisin variant described herein is a detergent composition containing polyethylene glycol and an organic acid, such as but limiting to, a detergent composition described in WO2021/041685.

In some embodiments, the cleaning composition comprising one or more subtilisin variant described herein is a detergent composition with effect on protein stains, such as but limiting to, a detergent composition described in U.S. Pat. No. 11,208,619.

In some embodiments, the cleaning composition comprising one or more subtilisin variant described herein is a detergent composition containing soil release polymers, such as but limiting to, a detergent composition described in US20220186144.

Examples of laundry detergent compositions include those provided in the Examples below, or in the following table:

Exemplary Liquid Laundry Detergent Composition

| Ingredient | % Active Matter |
|---|---|
| Sodium Dodecylbenzene Sulfonate | 7.3 |
| Sodium Laureth Sulfate | 6.3 |
| Potassium Cocoate | 1.05 |
| C12-15 Pareth-7 | 3.0 |
| Sodium Citrate | 2.65 |
| Ethanol | 1.0 |
| Propylene Glycol | 2.5 |
| Glycerol | 0.8 |
| Sorbitol | 0.8 |
| Triethanolamine | 0.5 |
| Nonionic polyester | 0.3 |
| Polycarboxylate | 1.38 |
| Polyvinylpyrrolidone | 0.08 |
| Methylisothiazolinone + Benzisothiazolinone | 0.01 |
| NaOH | 0.94 |
| Demin. Water | ad 100 |

In some embodiments, the cleaning composition is an ADW detergent composition comprising one or more subtilisin variant described herein. The ADW detergent composition can comprise two or more non-ionic surfactants selected from ethoxylated non-ionic surfactants, alcohol alkoxylated surfactants, epoxy-capped poly(oxyalkylated) alcohols, and amine oxide surfactants present in amounts from 0-10% by wt; builders in the range of 5-60% by wt. comprising either phosphate (mono-phosphates, di-phosphates, tri-polyphosphates or oligomeric-polyphosphates), sodium tripolyphosphate-STPP or phosphate-free builders (amino acid based compounds, e.g., MGDA (methyl-glycine-diacetic acid) and salts and derivatives thereof, GLDA (glutamic-N,N-diacetic acid) and salts and derivatives thereof, IDS (iminodisuccinic acid) and salts and derivatives thereof, carboxy methyl inulin and salts and derivatives thereof and mixtures thereof, nitrilotriacetic acid (NTA), diethylene triamine penta acetic acid (DTPA), and B-alaninediacetic acid (B-ADA) and their salts), homopolymers and copolymers of polycarboxylic acids and their partially or completely neutralized salts, monomeric polycarboxylic acids and hydroxycarboxylic acids and their salts in the range of 0.5-50% by wt; sulfonated/carboxylated polymers (provide dimensional stability to the product) in the range of about 0.1 to about 50% by wt; drying aids in the range of about 0.1 to about 10% by wt (selected from polyesters, especially anionic polyesters optionally together with further monomers with 3-6 functionalities which are conducive to polycondensation, specifically acid, alcohol or ester functionalities, polycarbonate-, polyurethane- and/or polyurea-polyorganosiloxane compounds or precursor compounds thereof of the reactive cyclic carbonate and urea type); silicates in the range from about 1 to about 20% by wt (sodium or potassium silicates, e.g., sodium disilicate, sodium meta-silicate and crystalline phyllosilicates); bleach-inorganic (e.g., perhydrate salts such as perborate, percarbonate, perphosphate, persulfate and persilicate salts) and organic (e.g., organic peroxyacids including diacyl and tetraacylperoxides, especially diperoxydodecanedioic acid, diperoxytetradecanedioic acid, and diperoxyhexadecanedioic acid); bleach activator-organic peracid precursors in the range from about 0.1 to about 10% by wt; bleach catalysts (selected from manganese triazacyclononane and related complexes, Co, Cu, Mn and Fe bispyridylamine and related complexes, and pentamine acetate cobalt (III) and related complexes); metal care agents in the range from about 0.1-5% by wt (selected from benzatriazoles, metal salts and complexes, and silicates); enzymes in the range from about 0.01-5.0 mg of active enzyme per gram of ADW detergent composition (acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, dispersins, endo-beta-1,4-glucanases, endo-beta-mannanases, esterases, exo-mannanases, galactanases, glucoamylases, hemicellulases, hexosaminidase, hyaluronidases, keratinases, laccases, lactases, ligninases, lipases, lipoxygenases, mannanases, nucleases, oxidases, oxidoreductases, pectate lyases, pectin acetyl esterases, pectinases, pentosanases, peroxidases, phenoloxidases, phosphatases, phospholipases, phytases, polyesterases, polygalacturonases, additional proteases, pullulanases, reductases, rhamnogalacturonases, beta-glucanases, tannases, transglutaminases, xanthan lyases, xylan acetyl-esterases, xylanases, xyloglucanases, xylosidases, and mixtures thereof); and enzyme stabilizer components (selected from oligosaccharides, polysaccharides and inorganic divalent metal salts).

Exemplary ADW compositions are provided in the Table below.

Exemplary ADW composition

| Ingredient | Weight in grams |
|---|---|
| Bleach Activator (tetraacetylethylenediamine; | 0.22 |
| TAED) | |
| SKS-6 sodium disilicate (Na₂Si₂O₅) | 0.8 |
| HEDP | 0.93 |
| Sodium carbonate | 1.5 |
| MGDA | 7.01 |
| Sulfonic acid group-containing polymer | 0.80 |
| (Acusol ™ 588) | |
| Sodium percarbonate | 3.50 |
| Bleach catalyst (Manganese 1,4,7- | 0.256 |
| triazacyclononane; MnTACN) | |
| LUTENSOL ® TO7 | 0.90 |
| PLURAFAC ® SLF 180 | 0.75 |
| Dipropylene glycol | 0.40 |
| Minors | balance |
| Total % of full dose | 100 |

More embodiments are directed to compositions and methods of treating fabrics (e.g., to desize a textile) using one or more subtilisin variant described herein. Fabric-treating methods are well known in the art (see, e.g., U.S. Pat. No. 6,077,316). For example, the feel and appearance of a fabric can be improved by a method comprising contacting the fabric with a variant described herein in a solution. The fabric can be treated with the solution under pressure.

One or more subtilisin variant described herein can be applied during or after weaving a textile, during the desizing stage, or one or more additional fabric processing steps. During the weaving of textiles, the threads are exposed to considerable mechanical strain. Prior to weaving on mechanical looms, warp yarns are often coated with sizing starch or starch derivatives to increase their tensile strength and to prevent breaking. One or more subtilisin variant described herein can be applied during or after weaving to remove the sizing starch or starch derivatives. After weaving, the variant can be used to remove the size coating before further processing the fabric to ensure a homogeneous and wash-proof result. One or more subtilisin variant described herein can be used alone or with other desizing chemical reagents and/or desizing enzymes to desize fabrics, including cotton-containing fabrics, as detergent additives, e.g., in aqueous compositions. An amylase also can be used in combination with the subtilisin variant in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of proteolytic enzymes to provide softness to the fabric and make the cotton more accessible to the subsequent enzymatic finishing steps. One or more subtilisin variant described herein can be used in methods of finishing denim garments (e.g., a “bio-stoning process”), enzymatic desizing and providing softness to fabrics, and/or finishing process.

The present disclosure also provides methods for cleaning a surface of an article, the method comprising contacting the article with at least one subtilisin variants provided herein (or a composition comprising such subtilisin variant). In some embodiments, the article may have a proteinaceous stain, for example, on its surface. In some embodiments, the proteinaceous stain may comprise egg or an egg-based stain, such as crème brûlée, baked cheese, BMI, or other protein-containing substance.

Non-limiting examples of compositions and methods disclosed herein are as follows:
1. A subtilisin variant comprising at least two, three or more substitutions selected from the group consisting of X9T, X17H, X45R, X68S, X78I, X86E, X87A, X96D, X100E, X100N, X103F, X103I, X108Q, X115L, X117R, X127S, X127T, X128K, X128P, X128R, X129Q, X155E, X161Q, X181E, X181Q, X202V, X203E, X203N, X217S, X221Q, X260W, and X264H, and wherein the positions are numbered according to SEQ ID NO: 1, and wherein the variant has at least 75% identity to the amino acid sequence of SEQ ID NO: 1 or SEQ ID NO: 8.
2. The subtilisin variant of embodiment 1, wherein the variant has a) at least 25% improved stability in detergent as compared to the parent subtilisin SEQ ID NO: 1; and or b) a net charge of −4 to +2 at pH 8 relative to the subtilisin having the amino acid sequence of SEQ ID NO: 1.
3. The subtilisin variant according to any of the preceding embodiments, wherein the variant further comprises one or more additional mutations selected from the group consisting of X24Q, X77N, X86D, X165Q, X184Q, X258D, and X258P, wherein the positions are numbered according to SEQ ID NO: 1.
4. The subtilisin variant of any of the preceding embodiments, wherein the variant comprises a set of substitutions selected from the group consisting of X9T-X17H, X9T-X45R, X9T-X68S, X9T-X78I, X9T-X86E, X9T-X87A, X9T-X96D, X9T-X100E, X9T-X100N, X9T-X103F, X9T-X103I, X9T-X108Q, X9T-X115L, X9T-X117R, X9T-X127S, X9T-X127T, X9T-X128K, X9T-X128P, X9T-X128R, X9T-X129Q, X9T-X155E, X9T-X161Q, X9T-X181E, X9T-X181Q, X9T-X202V, X9T-X203E, X9T-X203N, X9T-X217S, X9T-X260W, X9T-X221Q, X9T-X264H, X17H-X45R, X17H-X68S, X17H-X78I, X17H-X86E, X17H-X87A, X17H-X96D, X17H-X100E, X17H-X100N, X17H-X103F, X17H-X103I, X17H-X108Q, X17H-X115L, X17H-X117R, X17H-X127S, X17H-X127T, X17H-X128K, X17H-X128P, X17H-X128R, X17H-X129Q, X17H-X155E, X17H-X161Q, X17H-X181E, X17H-X181Q, X17H-X202V, X17H-X203E, X17H-X203N, X17H-X217S, X17H-X260W, X17H-X221Q, X17H-X264H, X45R-X68S, X45R-X78I, X45R-X86E, X45R-X87A, X45R-X96D, X45R-X100E, X45R-X100N, X45R-X103F, X45R-X103I, X45R-X108Q, X45R-X115L, X45R-X117R, X45R-X127S, X45R-X127T, X45R-X128K, X45R-X128P, X45R-X128R, X45R-X129Q, X45R-X155E, X45R-X161Q, X45R-X181E, X45R-X181Q, X45R-X202V, X45R-X203E, X45R-X203N, X45R-X217S, X45R-X260W, X45R-X221Q, X45R-X264H, X68S-X78I, X68S-X86E, X68S-X87A, X68S-X96D, X68S-X100E, X68S-X100N, X68S-X103F, X68S-X103I, X68S-X108Q, X68S-X115L, X68S-X117R, X68S-X127S, X68S-X127T, X68S-X128K, X68S-X128P, X68S-X128R, X68S-X129Q, X68S-X155E, X68S-X161Q, X68S-X181E, X68S-X181Q, X68S-X202V, X68S-X203E, X68S-X203N, X68S-X217S, X68S-X260W, X68S-X221Q, X68S-X264H, X78I-X86E, X78I-X87A, X78I-X96D, X78I-X100E, X78I-X100N, X78I-X103F, X78I-X103I, X78I-X108Q, X78I-X115L, X78I-X117R, X78I-X127S, X78I-X127T, X78I-X128K, X78I-X128P, X78I-X128R, X78I-X129Q, X78I-X155E, X78I-X161Q, X78I-X181E, X78I-X181Q, X78I-X202V, X78I-X203E, X78I-X203N, X78I-X217S, X78I-X260W, X78I-X221Q, X78I-X264H, X86E-X87A, X86E-X96D, X86E-X100E, X86E-X100N, X86E-X103F, X86E-X103I, X86E-X108Q, X86E-X115L, X86E-X117R, X86E-X127S, X86E-X127T, X86E-X128K, X86E-X128P, X86E-X128R, X86E-X129Q, X86E-X155E, X86E-X161Q, X86E-X181E, X86E-X181Q, X86E-X202V, X86E-X203E, X86E-X203N, X86E-X217S, X86E-X260W, X86E-X221Q, X86E-X264H, X87A-X96D, X87A-X100E, X87A-X100N, X87A-X103F, X87A-X103I, X87A-X108Q, X87A-X115L, X87A-X117R, X87A-X127S, X87A-X127T, X87A-X128K, X87A-X128P, X87A-X128R, X87A-X129Q, X87A-X155E, X87A-X161Q, X87A-X181E, X87A-X181Q, X87A-X202V, X87A-X203E, X87A-X203N, X87A-X217S, X87A-X260W, X87A-X221Q, X87A-X264H, X96D-X100E, X96D-X100N, X96D-X103F, X96D-X103I, X96D-X108Q, X96D-X115L, X96D-X117R, X96D-X127S, X96D-X127T, X96D-X128K, X96D-X128P, X96D-X128R, X96D-X129Q, X96D-X155E, X96D-X161Q, X96D-X181E, X96D-X181Q, X96D-X202V, X96D-X203E, X96D-X203N, X96D-X217S, X96D-X260W, X96D-X221Q, X96D-X264H, X100E-X100N, X100E-X103F, X100E-X103I, X100E-X108Q, X100E-X115L, X100E-X117R, X100E-X127S, X100E-X127T, X100E-X128K, X100E-X128P, X100E-X128R, X100E-X129Q, X100E-X155E, X100E-X161Q, X100E-X181E, X100E-X181Q, X100E-X202V, X100E-X203E, X100E-X203N, X100E-X217S, X100E-X260W, X100E-X221Q, X100E-X264H, X100N-X103F, X100N-X103I, X100N-X108Q, X100N-X115L, X100N-X117R, X100N-X127S, X100N-X127T, X100N-X128K, X100N-X128P, X100N-X128R, X100N-X129Q, X100N-X155E, X100N-X161Q, X100N-X181E, X100N-X181Q, X100N-X202V, X100N-X203E, X100N-X203N, X100N-X217S, X100N-X260W, X100N-X221Q, X100N-X264H, X103F-X103I, X103F-X108Q, X103F-X115L, X103F-X117R, X103F-X127S, X103F-X127T, X103F-X128K, X103F-X128P, X103F-X128R, X103F-X129Q, X103F-X155E, X103F-X161Q, X103F-X181E, X103F-X181Q, X103F-X202V, X103F-X203E, X103F-X203N, X103F-X217S, X103F-X260W, X103F-X221Q, X103F-X264H, X103I-X108Q, X103I-X115L, X103I-X117R, X103I-X127S, X103I-X127T, X103I-X128K, X103I-X128P, X103I-X128R, X103I-X129Q, X103I-X155E, X103I-X161Q, X103I-X181E, X103I-X181Q, X103I-X202V, X103I-X203E, X103I-X203N, X103I-X217S, X103I-X260W, X103I-X221Q, X103I-X264H, X108Q-X115L, X108Q-X117R, X108Q-X127S, X108Q-X127T, X108Q-X128K, X108Q-X128P, X108Q-X128R, X108Q-X129Q, X108Q-X155E, X108Q-X161Q, X108Q-X181E, X108Q-X181Q, X108Q-X202V, X108Q-X203E, X108Q-X203N, X108Q-X217S, X108Q-X260W, X108Q-X221Q, X108Q-X264H, X115L-X117R, X115L-X127S, X115L-X127T, X115L-X128K, X115L-X128P, X115L-X128R, X115L-X129Q, X115L-X155E, X115L-X161Q, X115L-X181E, X115L-X181Q, X115L-X202V, X115L-X203E, X115L-X203N, X115L-X217S, X115L-X260W, X115L-X221Q, X115L-X264H, X117R-X127S, X117R-X127T, X117R-X128K, X117R-X128P, X117R-X128R, X117R-X129Q, X117R-X155E, X117R-X161Q, X117R-X181E, X117R-X181Q, X117R-X202V, X117R-X203E, X117R-X203N, X117R-X217S, X117R-X260W, X117R-X221Q, X117R-X264H, X127S-X127T, X127S-X128K, X127S-X128P, X127S-X128R, X127S-X129Q, X127S-X155E, X127S-X161Q, X127S-X181E, X127S-X181Q, X127S-X202V, X127S-X203E, X127S-X203N, X127S-X217S, X127S-X260W, X127S-X221Q, X127S-X264H, X127T-X128K, X127T-X128P, X127T-X128R, X127T-X129Q, X127T-X155E, X127T-X161Q, X127T-X181E, X127T-X181Q, X127T-X202V, X127T-X203E, X127T-X203N, X127T-X217S, X127T-X260W, X127T-X221Q, X127T-X264H, X128K-X128P, X128K-X128R, X128K-X129Q, X128K-X155E, X128K-X161Q, X128K-X181E, X128K-X181Q, X128K-X202V, X128K-X203E, X128K-X203N, X128K-X217S, X128K-X260W, X128K-X221Q, X128K-X264H, X128P-X128R, X128P-X129Q, X128P-X155E, X128P-X161Q, X128P-X181E, X128P-X181Q, X128P-X202V, X128P-X203E, X128P-X203N, X128P-X217S, X128P-X260W, X128P-X221Q, X128P-X264H, X128R-X129Q, X128R-X155E, X128R-X161Q, X128R-X181E, X128R-X181Q, X128R-X202V, X128R-X203E, X128R-X203N, X128R-X217S, X128R-X260W, X128R-X221Q, X128R-X264H, X129Q-X155E, X129Q-X161Q, X129Q-X181E, X129Q-X181Q, X129Q-X202V, X129Q-X203E, X129Q-X203N, X129Q-X217S, X129Q-X260W, X129Q-X221Q, X129Q-X264H, X155E-X161Q, X155E-X181E, X155E-X181Q, X155E-X202V, X155E-X203E, X155E-X203N, X155E-X217S, X155E-X260W, X155E-X221Q, X155E-X264H, X161Q-X181E, X161Q-X181Q, X161Q-X202V, X161Q-X203E, X161Q-X203N, X161Q-X217S, X161Q-X260W, X161Q-X221Q, X161Q-X264H, X181E-X181Q, X181E-X202V, X181E-X203E, X181E-X203N, X181E-X217S, X181E-X260W, X181E-X221Q, X181E-X264H, X181Q-X202V, X181Q-X203E, X181Q-X203N, X181Q-X217S, X181Q-X260W, X181Q-X221Q, X181Q-X264H, X202V-X203E, X202V-X203N, X202V-X217S, X202V-X260W, X202V-X221Q, X202V-X264H, X203E-X203N, X203E-X217S, X203E-X260W, X203E-X221Q, X203E-X264H, X203N-X217S, X203N-X260W, X203N-X221Q, X203N-X264H, X217S-X260W, X217S-X221Q, X217S-X264H, X260W-X221Q, X260W-X264H, and X221Q-X264H, wherein the positions are numbered according to SEQ ID NO: 1.
5. The subtilisin variant of any of the preceding embodiments, wherein the variant comprises a set of substitutions selected from the group consisting of Q017H-N096D-G127T; Q017H-N096D-Y103F-A202V; G127T-A128K-S129Q-N184Q; P009T-Y103F-A202V-G203E; N096D-S100E-G127T-A202V; V087A-S155E-G165Q; P009T-T078I-Y103F-G127T; V087A-A202V-G203E; V045R-T161Q-S181Q-G203E; S155E-M221Q; T078I-Y103F; N096D-S100E-G127T-N217S; G127T-A128K-S129Q; T115L-G127T; P009T-T078I-N096D-A202V; V045R-S086E-S155E-A202V; V045R-T078I-Y103F-A202V; P009T-A202V-G203E-M221Q; A202V-G203E-M221Q-K264H; N096D-G127T; P009T-Q017H-Y103F-A202V; V045R-G127T-A128P-S129Q; S100E-G127T-A202V; A202V-M221Q; S100N-Y103I-G127T; G127T-A128K-S129Q-N217S; G127S-A128K-S129Q-A202V; T078I-N096D; A128K-S155E; N096D-Y103F-G165Q; S086E-T077N-T078I; V045R-T078I-S086E-A202V; N096D-S100E-G127S; T078I-G127T-A128K-S129Q; G127S-A128K-S129Q; Q017H-S086E-A202V-G203E; T161Q-S181Q-A202V-G203E; V045R-N096D-S100E; V045R-Y103F-G127T-A202V; N096D-S100E-G117R-G127T; V087A-G165Q-A202V; G127T-A128P-S129Q; V087A-M221Q; T115L-A202V-G203E; V045R-T078I-N096D-A202V; G127T-A128R-S129Q-G165Q; S100E-Y103I; S086E-S155E-A202V-G203E; N096D-Y103I-G127T; Q017H-N096D-Y103F; V045R-T078I-N096D-Y103F; S108Q-G127T; T078I-S086E-A202V-G203E; T078I-Y103F-G127T-A202V; S181E-A202V-G203E-S258D; N096D-A202V-G203E; N096D-Y103I-G127T-M221Q; V087A-G165Q-A202V-G203E; P009T-G127T-A128K-S129Q; S100E-G127T; S100N-M221Q; G127T-A128K-S129Q-A202V; V087A-S100N; S100E-G117R-G127T; T077N-G127T-A128K-S129Q; Q017H-N096D-G127T-A202V; N096D-A202V-M221Q; V045R-N096D-Y103F-G127T; N096D-G127T-G203E; V045R-S086E-A202V-G203E; T078I-S086E-S155E-G203E; V045R-M221Q; Q017H-N096D-Y103F-G127T; Q017H-Y103F-G127T-A202V; T078I-A202V-M221Q; V045R-N096D-G127T; V045R-N096D-G127T-A202V; P009T-T078I-Y103F-A202V; S100N-T115L; S086E-A202V; N096D-Y103F-G127T; T078I-N096D-G127T; T115L-G165Q-A202V; T078I-S086E; V045R-T078I-N096D-G127T; P009T-N096D-G127T; G127T-A128R-S129Q-A202V; T078I-N096D-A202V; N096D-Y103I-G203E; G127S-A128P-S129Q-A202V; N096D-G127T-N217S; V045R-T078I; A024Q-T078I-A202V; A202V-G203N; P009T-T077N-T078I; G203E-K264H; T077N-T078I-G165Q-A202V; V045R-T077N-G165Q-A202V; G165Q-A202V-G203E-S258P; P009T-T078I-A202V; V045R-G127T; T077N-T078I-G165Q-G203E; V045R-T078I-A202V-G203E; G127T-G165Q-A202V-G203N; A202V-K264H; P009T-A202V-G203E; T077N-A202V-G203E; A202V-G203E-N217S; Y103I-A202V; T078I-G203N; P009T-Q017H-T078I-G165Q; Q017H-T078I; S086D-A202V-G203E; A202V-G203E-F260W; G165Q-A202V-G203E-N217S; G127T-G165Q-A202V-G203E; P009T-Q017H-T078I-G127T; V045R-S155E; P009T-Q017H-T078I-A202V; P009T-T078I; S086D-S155E-A202V-G203E; P009T-Q017H-A202V-G203E; A024Q-G165Q-A202V-G203E; P009T-Q017H-T077N-A202V; Q017H-A202V-G203E; P009T-G127T-A202V-G203E; Y103I-G203E; T078I-S155E-A202V-G203E; N096D-S100E-Y103I; Q017H-T078I-G127T-A202V; P009T-N096D; A128R-S155E; G127T-A128R; T078I-A202V; P009T-A202V; Q017H-A202V; P009T-G203E; T078I-G165Q-A202V; T078I-A202V-G203E-N217S; and T077N-T078I-M221Q; wherein the positions are numbered according to SEQ ID NO: 1.
6. The subtilisin variant of any preceding embodiment, wherein said variant comprises an amino acid sequence with 80%, 85%, 86%, 87%, 88%, 89%, 90%, 91%, 92%, 93%, 94%, 95%, 96%, 97%, 98%, 99% or less than 100% amino acid sequence identity to SEQ ID NO: 1.
7. The subtilisin variant of any preceding embodiment, wherein said variant has one or more improved property when compared to a parent or reference subtilisin; wherein the improved property is selected from improved cleaning performance in detergent, improved stability in detergent; improved aged cleaning performance, and combinations thereof.
8. The subtilisin variant of embodiment 7, wherein the improved property is
(i) an improved cleaning performance in detergent, wherein said variant has a blood aged on cotton (CS-01) cleaning PI≥1.1 compared to the subtilisin having the amino acid sequence of SEQ ID NO: 8; and/or,
(ii) an improved cleaning performance in detergent, wherein said variant has a chocolate rice pudding aged on cotton (CS-100) cleaning PI≥1.1 compared to the subtilisin having the amino acid sequence of SEQ ID NO: 8; and/or
(iii) an improved cleaning performance in detergent, wherein said variant has a full egg with carbon black aged on cotton (C-S-39) cleaning PI≥1.1 compared to the subtilisin having the amino acid sequence of SEQ ID NO: 8; and/or,
(iv) an improved cleaning performance in detergent, wherein said variant has a chocolate soymilk drink aged on cotton (C-S-45) cleaning PI≥1.1 compared to the subtilisin having the amino acid sequence of SEQ ID NO: 8; and/or,
(v) an improved cleaning performance in detergent, wherein said variant has a grass on cotton (CS-07) cleaning PI≥1.1 compared to the subtilisin having the amino acid sequence of SEQ ID NO: 8; and/or,
(vi) an improved cleaning performance in detergent, wherein said variant has a chocolate milk with carbon black on cotton (C-03) cleaning PI≥1.1 compared to the subtilisin having the amino acid sequence of SEQ ID NO: 8; and/or,
(vii) an improved cleaning performance in detergent, wherein said variant has a blood/milk/ink on woven cotton (CS-05) cleaning PI≥1.1 compared to the subtilisin having the amino acid sequence of SEQ ID NO: 8; and/or,
(viii) an improved cleaning performance in detergent, wherein said variant has a milk with carbon black on cotton (C-11) cleaning PI≥1.1 compared to the subtilisin having the amino acid sequence of SEQ ID NO: 8; and/or,
(ix) an improved cleaning performance in detergent, wherein said variant has a blood/milk/ink on polycotton (EMPA 116) cleaning PI≥1.1 compared to the subtilisin having the amino acid sequence of SEQ ID NO: 8; and/or,
(x) an improved cleaning performance in detergent, wherein said variant has a blood aged on polyester/cotton (KCS-01) cleaning PI≥1.1 compared to the subtilisin having the amino acid sequence of SEQ ID NO: 8; and/or,
(xi) an improved stability, wherein said variant has a higher residual activity compared to the subtilisin having the amino acid sequence of SEQ ID NO: 1 or SEQ ID NO: 8 when measured in accordance with the stability assay of Example 2; and/or,
(xii) an improved aged cleaning performance measured as the difference in stain removal measured for a sample of aged test sample (where the enzyme is pre-incubated in detergent for an extended period of time such as 3-8 weeks at an elevated temperature such as 37° C.) compared to the aged cleaning performance of SEQ ID NO: 8.
9. The subtilisin variant of embodiment 7, wherein the improved property is
(i) proteolytic activity on DMC assay; and/or,
(ii) an improved stability in detergent, wherein said variant has a higher residual activity compared to the subtilisin having the amino acid sequence of SEQ ID NO: 1; and/or, (iii) an improved cleaning performance in detergent, wherein said variant has a blood/milk/ink on polycotton (EMPA 116) cleaning PI≥1.1 compared to the subtilisin having the amino acid sequence of SEQ ID NO: 1,
(iv) a net charge at pH 8 between-4 and +2,
and wherein the subtilisin variant has at least 75% sequence identity to SEQ ID NO:1.
10. An enzyme composition comprising one or more subtilisin variant according to any preceding embodiment.
11. The enzyme composition according to embodiment 10, further comprising one or more other enzymes selected from acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, dispersins, endo-beta-1,4-glucanases, endo-beta-mannanases, esterases, exo-mannanases, galactanases, glucoamylases, hemicellulases, hexosaminidase, hyaluronidases, keratinases, laccases, lactases, ligninases, lipases, lipoxygenases, lysozymes, mannanases, metalloproteases, nucleases (e.g. DNases and/or RNases), oxidases, oxidoreductases, pectate lyases, pectin acetyl esterases, pectinases, pentosanases, perhydrolases, peroxidases, phenoloxidases, phosphatases, phospholipases, phosphodiesterases, phytases, polygalacturonases, polyesterases, additional proteases, pullulanases, reductases, rhamnogalacturonases, beta-glucanases, tannases, transglutaminases, xanthan lyases, xylan acetyl-esterases, xylanases, xyloglucanases, xylosidases, and any combination or mixture thereof.
12. A polynucleotide comprising a nucleic acid sequence encoding a variant of any one of Embodiments 1-9, wherein said polynucleotide is, optionally, isolated.
13. The polynucleotide of embodiment 12, wherein the nucleic acid sequence is operably linked to a promoter.
14. An expression vector or cassette comprising the polynucleotide of Embodiment 12.
15. A recombinant host cell comprising the polynucleotide of Embodiment 12.
16. A cleaning composition comprising the subtilisin variant of any one of embodiments 1-9 and at least one adjunct.
17. The cleaning composition of embodiment 16, wherein the cleaning composition is a composition selected from the group consisting of a laundry detergent, a fabric softening detergent, a dishwashing detergent (e.g., automatic or hand dishwashing detergents), a hard-surface cleaning detergent, and a medical instrument cleaning composition.
18. The cleaning composition of embodiments 16-17, wherein the detergent composition does not comprise a synthetic or peptidic protease stabilizer.
19. A method of cleaning comprising, contacting a surface or an item in need of cleaning with an effective amount of a subtilisin variant of any one of embodiments 1-9 or the enzyme composition of any one of embodiments 10-11 or 16-17; and optionally further comprising the step of rinsing said surface or item after contacting said surface or item with said variant or enzyme composition.
20. The method of embodiment 19, wherein said item is dishware, or fabric, or a medical instrument.

The following examples are provided to demonstrate and illustrate certain preferred embodiments and aspects of the present disclosure and should not be construed as limiting.

### Example 1

### Expression of AprL Subtilisin Variants

The Bacillus licheniformis subtilisin (AprL) is provided in SEQ ID NO: 1. This AprL subtilisin wild type sequence was used as the starting point for protein engineering. All AprL subtilisin variants were expressed using a DNA fragment comprising: a 5′ AprE flanking region that contains a variant of the B. subtilis rrnIp2 promoter sequence (SEQ ID NO:2) (the B. subtilis rrnIp2 promoter and engineered variant are more fully described in patent application WO2020112609), the nucleotide sequence encoding the aprE signal peptide sequence (SEQ ID NO: 3), the nucleotide sequence encoding the B. licheniformis AprL propeptide (SEQ ID NO: 4), the sequence corresponding to the gene encoding the mature AprL subtilisin (SEQ ID NO: 5), the Bacillus amyloliquefaciens (BPN′) terminator and flanking sequence (SEQ ID NO: 6), the 3′ AprE flanking sequences including a kanamycin gene expression cassette (SEQ ID NO: 7), in consecutive order. This DNA fragment was assembled using standard molecular biology techniques. Linear DNA of expression cassettes were used to transform competent B. subtilis cells of a suitable strain.

The transformation mixtures were plated onto LA plates containing 1.6% skim milk and 5 ppm kanamycin and incubated overnight at 37° C. Single colonies were picked and grown in Luria broth at 37° C. under antibiotic selection. For protein studies, transformed cells were grown in 96-well microtiter plates (MTPs) in cultivation medium (enriched semi-defined media based on MOPS buffer, with urea as major nitrogen source, glucose as the main carbon source, supplemented with 1% soytone for robust cell growth, containing antibiotic selection) for 3 days at 37° C., 300 rpm, with 80% humidity in a shaking incubator. After centrifugation and filtration, clarified culture supernatants containing the proteases of interest were used for assays.

Alternatively, AprL protease variants were also expressed by integrating an expression cassette for the protease gene into B. licheniformis serA locus in a suitable B. licheniformis strain for protein expression. AprL subtilisin variants were expressed using DNA fragments comprising of a 5′ serA flanking sequence (SEQ ID NO: 9) containing a variant of the B. subtilis rrnIp2 promoter sequence (SEQ ID NO: 16) (see WO2020112609) operably linked to the DNA encoding B. subtilis aprE 5′ UTR (SEQ ID NO: 10), operably linked to the DNA encoding the aprL signal peptide (SEQ ID NO: 11), operably linked to the DNA encoding the AprL propeptide (SEQ ID NO: 4) operably linked to the DNA encoding the mature AprL subtilisin or variant of interest, the B. licheniformis amyL terminator (SEQ ID NO: 12), operably linked to the 3′ serA flanking sequence (SEQ ID NO: 13). A second DNA expression cassette comprising the 5′ lysA flanking sequence (SEQ ID NO: 14) containing a variant of the B. subtilis rrnIp2 promoter sequence (SEQ ID NO: 17), operably linked to the DNA encoding the B. subtilis aprE 5′ UTR (SEQ ID NO: 10), operably linked to the DNA encoding the aprL signal peptide (SEQ ID NO: 11), operably linked to the DNA encoding the AprL propeptide (SEQ ID NO: 4), operably linked to the DNA encoding the mature AprL subtilisin or variant of interest, operably linked to the amyL terminator (SEQ ID NO: 12), operably linked to the lysA 3′ flanking region (SEQ ID NO: 15).

Recombinant B. licheniformis cells can be constructed in various ways, for example, using linear fragments in combination with pBl.comK (ref. WO2019/40412). Briefly, B. licheniformis cells containing pBl.comK were made competent by methods known in the art (e.g. WO2021/146411). One hundred microliters of competent cells were mixed with 10 ul of a linear DNA fragment. The mixture was incubated at 37° C. at 1400 rpm for 1.5 hours. The mixture was plated on selective medium (eg. Minimal medium lacking lysine or serine) to isolate strains transformed with the fragment of interest.

In some instances, the variant proteases very isolated from B licheniformis cultures grown in flat bottom shake flasks, at 37° C. for approximately 86 hours in humidified incubator. For some strains, a MES-based media containing soytone and essential salts and minerals was used, and in other instances, a MOPS-based media containing soytone and essential salts and minerals was used. To harvest protein of interest for stability and performance studies, cultivation broth was centrifuged 30 min at 4000 rpm and 4° C. Filtration over 0.45 uM and 0.2 uM filters resulted in clarified supernatant that was frozen and then transfer to −80° C. for sample storage.

### Example 2

### Assays

### Protein Determination

The concentration of the AprL subtilisin variants in culture supernatant was determined by UHPLC using a Zorbax 300 SB-C3 column and linear gradient of 0.1% Trifluoroacetic acid (Solution A) and 0.07% Trifluoroacetic acid in Acetonitrile (Solution B) and detection at 220 nm. Culture supernatants were diluted in 10 mM NaCl, 0.1 mM CaCl₂), 0.005% Tween®-80 for loading onto column. The protein concentration of the samples was calculated using a standard curve of the purified reference enzyme (SEQ ID NO:8).

### Protease Activity

The protease activity of AprL subtilisin variants was tested by measuring the hydrolysis of AAPF-pNA synthetic peptidic substrate, or activity on di-methyl casein substrate.

For the AAPF assay, the reagent solutions used were: 100 mM Tris pH 8.6, 0.005% Tween®-80 (Tris buffer) and 160 mM suc-AAPF-pNA in DMSO (suc-AAPF-pNA stock solution) (Sigma: S-7388). To prepare a working solution, 1 mL suc-AAPF-pNA stock solution was added to 100 mL Tris buffer and mixed. An enzyme sample was added to a microtiter plate (MTP) containing 1 mg/mL suc-AAPF-pNA working solution and assayed for activity at 405 nm over 3-5 min using a SpectraMax plate reader in kinetic mode at room temperature (RT). The protease activity was expressed as mOD/min.

### Stability Assay in Liquid Laundry Detergent

The liquid laundry detergents used in stability assays were Persil Small & Mighty Non-Bio Liquid Detergent “Persil Non-Bio” (PNB, Unilever), Chinese National Standard HDL (CNS, Table 1) and Test Detergent A (Table 2).

TABLE 1
CNS liquid laundry detergent composition

| | Percent (%) w/w active |
|---|---|
| Component | matter |
| LAS | 8 |
| AES | 2 |
| AEO (EO═9) | 4 |
| Triethanolamine | 0.5 |
| Sodium citrate | 0.5 |
| NaOH | To adjust pH to 8.5-9.0 |
| Water | up to 100 |

TABLE 2
Test detergent A composition

| Ingredient | Active matter % |
|---|---|
| Biosoft ® LA Acid | 7.30 |
| Wilfares ® ESB 70 | 6.30 |
| NANSA ® PC 38/F | 1.05 |
| Tomadol ® 25-7 | 3.00 |
| Sodium Citrate | 2.65 |
| Ethanol | 1.00 |
| Propylene Glycol | 2.50 |
| Glycerol | 0.80 |
| Sorbitol | 0.80 |
| Triethanolamine | 0.50 |
| TexCare SRN 260 liquid | 0.30 |
| ACUSOL ™ 420N powder | 1.38 |
| PVP K-30 liquid | 0.08 |
| ROCIMA ™ MBX Biocide | 0.01 |
| NaOH | 0.94 |
| Demin. Water | Ad 100 |

The stability of the subtilisin variants described herein was measured by diluting the variants in 10% or 20% (v/v) detergent solution and measuring the proteolytic activity of the variants before and after a heat incubation step using the AAPF assay described above. The test samples were incubated at 45°−75° C. for 20 min in a 384-well thermocycler, and the stability results were calculated as the fraction of remaining activity for each enzyme sample by taking the ratio of mOD/min for stressed over unstressed condition.

### Example 3

### Stability in Detergent of AprL Variants

The stability of AprL variants generated in this study were evaluated in several liquid laundry detergents using the methods described in Example 2. Tables 3, 4, and 5 show results for enzyme samples generated from expression in B. subtilis host using the method described in Example 1, obtained as clarified supernatant from 96 well cultures. The AprL-WT subtilisin (SEQ ID NO:1) and the variant subtilisin CMT24 (SEQ ID NO:8) were included for comparison.

TABLE 3
Stability evaluation of AprL variants reported as
fraction of residual activity when tested under
conditions (temperature and detergent) described.

| | | 10% | |
|---|---|---|---|
| Enzyme | | PNB | 20% CNS |
| Name | Substitutions vs AprL | 55° C. | 58° C. |
| AprL-WT | none | 0.05 | <0.05 |
| CMT24 | A068S-T077N-T078I-G127S- | 0.76 | 0.59 |
| | A128P-G165Q-N184Q-A202V- | | |
| | N217S-S258P | | |
| CMT24-04386 | V045R-T078I-S086E-Y103F- | 0.78 | 0.52 |
| | A128P-G165Q-G203E-S258D | | |
| CMT24-04389 | Q017H-A024Q-S086D-Y103F- | 0.79 | 0.59 |
| | A128P-S129Q-G165Q-G203E- | | |
| | S258P | | |
| CMT24-04427 | Q017H-V045R-S086D-Y103I- | 0.80 | 0.54 |
| | S129Q-G165Q-A202V-S258D | | |
| CMT24-04428 | Q017H-A024Q-V045R-T078I- | 0.85 | 0.56 |
| | S086D-Y103F-A128P-S129Q- | | |
| | G165Q-G203N-S258D | | |
| CMT24-04503 | T078I-S086E-Y103I-A128P- | 0.85 | 0.42 |
| | S129Q-G203E-S258P | | |

TABLE 4
Stability evaluation of AprL variants reported as
fraction of residual activity when tested under
conditions (temperature and detergent) described.

| | | 10% | 20% |
|---|---|---|---|
| Enzyme | | PNB | CNS |
| Name | Substitutions vs AprL | 69° C. | 65° C. |
| AprL-WT | none | 0.00 | 0.00 |
| CMT24 | A068S-T077N-T078I-G127S- | 0.09 | 0.21 |
| | A128P-G165Q-N184Q-A202V- | | |
| | N217S-S258P | | |
| CMT24-02741 | T077N-T078I-N096D-G127S- | 0.43 | 0.48 |
| | A128P-G165Q-N184Q-A202V- | | |
| | G203E-N217S-S258P | | |
| CMT24-02769 | P009T-T077N-T078I-N096D- | 0.23 | 0.53 |
| | G127T-A128P-G165Q-N184Q- | | |
| | A202V-N217S-S258P | | |
| CMT24-02767 | T077N-T078I-N096D-G127T- | 0.39 | 0.54 |
| | A128P-G165Q-N184Q-A202V- | | |
| | G203E-N217S-S258P | | |
| CMT24-02704 | T077N-T078I-N096D-S108Q- | 0.50 | 0.51 |
| | G127S-A128P-G165Q-N184Q- | | |
| | A202V-G203E-N217S-S258P | | |
| CMT24-02712 | P009T-T077N-T078I-N096D- | 0.57 | 0.72 |
| | G127S-A128P-G165Q-N184Q- | | |
| | A202V-G203E-N217S-S258P | | |
| CMT24-02688 | Q017H-T077N-T078I-N096D- | 0.66 | 0.87 |
| | G127S-A128P-G165Q-N184Q- | | |
| | A202V-G203E-N217S-S258P | | |
| CMT24-02954 | P009T-T077N-T078I-G127T- | 0.61 | 0.66 |
| | A128P-S129Q-G165Q-N184Q- | | |
| | A202V-G203E-N217S-S258P | | |
| CMT24-02839 | P009T-T077N-T078I-N096D- | 0.68 | 0.77 |
| | G127T-A128P-G165Q-N184Q- | | |
| | A202V-G203E-N217S-S258P | | |
| CMT24-02732 | Q017H-T077N-T078I-N096D- | 0.68 | 0.68 |
| | G127T-A128P-G165Q-N184Q- | | |
| | A202V-G203E-N217S-S258P | | |
| CMT24-03776 | T077N-T078I-S086D-N096D- | 0.42 | 0.75 |
| | Y103F-G127S-A128R-G165Q- | | |
| | N184Q-A202V-N217S-S258P | | |
| CMT24-02716 | T077N-T078I-N096D-S108Q- | 0.37 | 0.43 |
| | G127T-A128P-G165Q-N184Q- | | |
| | A202V-G203E-N217S-S258P | | |
| CMT24-03823 | Q017H-T077N-T078I-N096D- | 0.37 | 0.43 |
| | G127T-A128R-G165Q-N184Q- | | |
| | A202V-G203E-N217S-S258P | | |
| CMT24-03165 | P009T-T077N-T078I-G127T- | 0.87 | 0.73 |
| | A128K-S129Q-G165Q-N184Q- | | |
| | A202V-G203E-N217S-S258D | | |
| CMT24-02725 | A068S-T077N-T078I-N096D- | 0.49 | 0.63 |
| | S108Q-G127S-A128P-G165Q- | | |
| | N184Q-A202V-G203E-N217S- | | |
| | S258P | | |
| CMT24-02776 | Q017H-A068S-T077N-T078I- | 0.32 | 0.65 |
| | N096D-S108Q-G127T-A128P- | | |
| | G165Q-N184Q-A202V-N217S- | | |
| | S258P | | |
| CMT24-03121 | Q017H-A068S-T077N-T078I- | 0.41 | 0.47 |
| | S108Q-G127T-A128P-S129Q- | | |
| | G165Q-N184Q-A202V-N217S- | | |
| | S258P | | |
| CMT24-02633 | P009T-V045R-T077N-T078I- | 0.48 | 0.59 |
| | N096D-G127S-A128P-G165Q- | | |
| | N184Q-A202V-G203E-N217S- | | |
| | S258P | | |
| CMT24-02783 | P009T-Q017H-T077N-T078I- | 0.90 | 0.89 |
| | N096D-G127S-A128P-G165Q- | | |
| | N184Q-A202V-G203E-N217S- | | |
| | S258P | | |
| CMT24-02672 | Q017H-T077N-T078I-N096D- | 0.70 | 0.82 |
| | S108Q-G127S-A128P-G165Q- | | |
| | N184Q-A202V-G203E-N217S- | | |
| | S258P | | |
| CMT24-02650 | P009T-T077N-T078I-N096D- | 0.64 | 0.79 |
| | S108Q-G127S-A128P-G165Q- | | |
| | N184Q-A202V-G203E-N217S- | | |
| | S258P | | |
| CMT24-02721 | Q017H-V045R-T077N-T078I- | 0.58 | 0.74 |
| | N096D-G127S-A128P-G165Q- | | |
| | N184Q-A202V-G203E-N217S- | | |
| | S258P | | |
| CMT24-02710 | P009T-V045R-T077N-T078I- | 0.67 | 0.53 |
| | N096D-G127T-A128P-G165Q- | | |
| | N184Q-A202V-G203E-N217S- | | |
| | S258P | | |
| CMT24-02971 | P009T-V045R-T077N-T078I- | 0.45 | 0.45 |
| | G127T-A128P-S129Q-G165Q- | | |
| | N184Q-A202V-G203E-N217S- | | |
| | S258P | | |
| CMT24-02937 | P009T-T077N-T078I-N096D- | 0.36 | 0.61 |
| | G127T-A128P-S129Q- | | |
| | G165Q-N184Q-A202V-G203N- | | |
| | N217S-S258P | | |
| CMT24-02693 | P009T-T077N-T078I-N096D- | 0.66 | 0.73 |
| | S108Q-G127T-A128P-G165Q- | | |
| | N184Q-A202V-G203E-N217S- | | |
| | S258P | | |
| CMT24-02907 | P009T-T077N-T078I-Y103F- | 0.73 | 0.71 |
| | G127T-A128P-S129Q-G165Q- | | |
| | N184Q-A202V-G203E-N217S- | | |
| | S258P | | |
| CMT24-02765 | Q017H-V045R-T077N-T078I- | 0.55 | 0.73 |
| | N096D-G127T-A128P- | | |
| | G165Q-N184Q-A202V-G203E- | | |
| | N217S-S258P | | |
| CMT24-02636 | Q017H-T077N-T078I-N096D- | 0.67 | 0.70 |
| | S108Q-G127T-A128P- | | |
| | G165Q-N184Q-A202V-G203E- | | |
| | N217S-S258P | | |
| CMT24-02974 | Q017H-T077N-T078I-N096D- | 0.32 | 0.55 |
| | S108Q-G127T-A128P- | | |
| | G165Q-N184Q-A202V-G203N- | | |
| | N217S-S258P | | |
| CMT24-03368 | P009T-T077N-T078I-Y103F- | 0.71 | 0.70 |
| | G127T-A128K-S129Q- | | |
| | G165Q-N184Q-A202V-G203E- | | |
| | N217S-S258P | | |
| CMT24-03333 | T077N-T078I-N096D-Y103F- | 0.54 | 0.48 |
| | G127T-A128K-S129Q- | | |
| | G165Q-N184Q-A202V-G203E- | | |
| | N217S-S258P | | |
| CMT24-03367 | T077N-T078I-S108Q-T115L- | 0.39 | 0.36 |
| | G127T-A128K-S129Q- | | |
| | G165Q-N184Q-A202V-G203E- | | |
| | N217S-S258P | | |
| CMT24-03282 | P009T-T077N-T078I-N096D- | 0.64 | 0.69 |
| | G127T-A128K-S129Q- | | |
| | G165Q-N184Q-A202V-G203E- | | |
| | N217S-S258P | | |
| CMT24-03360 | T077N-T078I-S108Q-T115L- | 0.31 | 0.27 |
| | G127T-A128K-S129Q- | | |
| | G165Q-N184Q-A202V-N217S- | | |
| | S258P-K264H | | |
| CMT24-03203 | T077N-T078I-Y103F-S108Q- | 0.48 | 0.45 |
| | G127T-A128K-S129Q- | | |
| | G165Q-N184Q-A202V-G203E- | | |
| | N217S-S258P | | |
| CMT24-03185 | P009T-T077N-T078I-S108Q- | 0.84 | 0.64 |
| | G127T-A128K-S129Q- | | |
| | G165Q-N184Q-A202V-G203E- | | |
| | N217S-S258D | | |
| CMT24-03231 | T077N-T078I-S108Q-T115L- | 0.36 | 0.44 |
| | G127T-A128K-S129Q- | | |
| | G165Q-N184Q-A202V-G203E- | | |
| | N217S-S258D | | |
| CMT24-02850 | P009T-A068S-T077N-T078I- | 0.40 | 0.59 |
| | N096D-Y103F-G127S- | | |
| | A128P-S129Q-G165Q-N184Q- | | |
| | A202V-N217S-S258P | | |
| CMT24-02823 | Q017H-A068S-T077N-T078I- | 0.70 | 0.82 |
| | N096D-S108Q-G127T- | | |
| | A128P-G165Q-N184Q-A202V- | | |
| | G203E-N217S-S258P | | |
| CMT24-02830 | Q017H-V045R-T077N-T078I- | 0.61 | 0.66 |
| | N096D-S108Q-G127S- | | |
| | A128P-G165Q-N184Q-A202V- | | |
| | G203E-N217S-S258P | | |
| CMT24-03862 | A068S-T077N-T078I-S086D- | 0.77 | 0.98 |
| | T115L-G127T-A128P- | | |
| | S129Q-G165Q-N184Q-A202V- | | |
| | G203E-N217S-S258D | | |
| CMT24-03077 | P009T-V045R-T077N-T078I- | 0.50 | 0.57 |
| | N096D-G127S-A128P- | | |
| | S129Q-G165Q-N184Q-A202V- | | |
| | G203E-N217S-S258P | | |
| CMT24-02906 | P009T-V045R-T077N-T078I- | 0.56 | 0.59 |
| | S108Q-G127S-A128P- | | |
| | S129Q-G165Q-N184Q-A202V- | | |
| | G203E-N217S-S258P | | |
| CMT24-03117 | P009T-Q017H-T077N-T078I- | 1.01 | 0.88 |
| | Y103F-G127S-A128P- | | |
| | S129Q-G165Q-N184Q-A202V- | | |
| | G203E-N217S-S258P | | |
| CMT24-02825 | P009T-V045R-T077N-T078I- | 0.50 | 0.52 |
| | N096D-S108Q-G127T- | | |
| | A128P-G165Q-N184Q-A202V- | | |
| | G203E-N217S-S258P | | |
| CMT24-02861 | Q017H-V045R-T077N-T078I- | 0.75 | 0.75 |
| | N096D-G127T-A128P- | | |
| | G165Q-N184Q-A202V-G203E- | | |
| | N217S-S258P-K264H | | |
| CMT24-03264 | P009T-A068S-T077N-T078I- | 0.36 | 0.39 |
| | S108Q-T115L-G127T- | | |
| | A128K-S129Q-G165Q-N184Q- | | |
| | A202V-N217S-S258D | | |
| CMT24-03521 | A024Q-V045R-T077N-T078I- | 0.79 | 0.86 |
| | S086D-G127T-A128P- | | |
| | S129Q-G165Q-N184Q-A202V- | | |
| | G203E-N217S-S258P | | |
| CMT24-03701 | Q017H-V045R-T077N-T078I- | 0.38 | 0.89 |
| | S086D-S108Q-G127T- | | |
| | A128P-S129Q-G165Q-N184Q- | | |
| | A202V-N217S-S258P | | |
| CMT24-03569 | Q017H-V045R-T077N-T078I- | 0.50 | 0.75 |
| | S086D-T115L-G127T- | | |
| | A128P-S129Q-G165Q-N184Q- | | |
| | A202V-N217S-S258P | | |
| CMT24-03471 | Q017H-T077N-T078I-Y103F- | 0.67 | 0.63 |
| | S108Q-G127T-A128K- | | |
| | S129Q-G165Q-N184Q-A202V- | | |
| | G203E-N217S-S258P | | |
| CMT24-03217 | P009T-V045R-T077N-T078I- | 0.53 | 0.53 |
| | N096D-G127T-A128K- | | |
| | S129Q-G165Q-N184Q-A202V- | | |
| | G203E-N217S-S258P | | |
| CMT24-03800 | P009T-Q017H-T077N-T078I- | 0.64 | 0.97 |
| | Y103F-G127S-A128K- | | |
| | S129Q-G165Q-N184Q-A202V- | | |
| | N217S-S258D-K264H | | |
| CMT24-03289 | Q017H-T077N-T078I-S108Q- | 0.68 | 0.80 |
| | T115L-G127T-A128K- | | |
| | S129Q-G165Q-N184Q-A202V- | | |
| | G203E-N217S-S258P | | |
| CMT24-03276 | Q017H-T077N-T078I-Y103F- | 0.41 | 0.82 |
| | T115L-G127T-A128K- | | |
| | S129Q-G165Q-N184Q-A202V- | | |
| | G203E-N217S-S258P | | |
| CMT24-03741 | Q017H-A024Q-T077N-T078I- | 0.75 | 0.88 |
| | N096D-Y103F-G127S- | | |
| | A128K-G165Q-N184Q-A202V- | | |
| | G203N-N217S-S258D | | |
| CMT24-03192 | Q017H-V045R-T077N-T078I- | 0.53 | 0.61 |
| | N096D-G127T-A128K- | | |
| | S129Q-G165Q-N184Q-A202V- | | |
| | G203E-N217S-S258P | | |
| CMT24-03275 | Q017H-T077N-T078I-Y103F- | 0.76 | 0.79 |
| | G127T-A128K-S129Q- | | |
| | G165Q-N184Q-A202V-G203E- | | |
| | N217S-S258P-K264H | | |
| CMT24-03757 | T077N-T078I-S086D-N096D- | 0.38 | 0.65 |
| | Y103F-S108Q-G117R- | | |
| | G127T-A128K-G165Q-N184Q- | | |
| | A202V-N217S-S258P | | |
| CMT24-03205 | P009T-T077N-T078I-N096D- | 0.55 | 0.54 |
| | Y103F-G127T-A128K- | | |
| | S129Q-G165Q-N184Q-A202V- | | |
| | N217S-S258P-K264H | | |
| CMT24-03809 | V045R-T077N-T078I-S086D- | 0.32 | 0.69 |
| | N096D-S108Q-G127T- | | |
| | A128K-S129Q-G165Q-N184Q- | | |
| | A202V-N217S-S258P | | |
| CMT24-03561 | V045R-T077N-T078I-N096D- | 0.32 | 0.38 |
| | S108Q-G127T-A128P- | | |
| | G165Q-N184Q-A202V-G203E- | | |
| | N217S-S258D-K264H | | |
| CMT24-03258 | P009T-Q017H-T077N-T078I- | 0.50 | 0.68 |
| | Y103F-T115L-G127T- | | |
| | A128K-S129Q-G165Q-N184Q- | | |
| | A202V-N217S-S258D | | |
| CMT24-03658 | P009T-T077N-T078I-S086D- | 0.65 | 1.10 |
| | N096D-G127T-A128K- | | |
| | S129Q-G165Q-N184Q-A202V- | | |
| | N217S-S258D-K264H | | |
| CMT24-02880 | Q017H-A068S-T077N-T078I- | 0.50 | 0.75 |
| | N096D-Y103F-S108Q- | | |
| | G127S-A128P-G165Q-N184Q- | | |
| | A202V-G203N-N217S- | | |
| | S258P | | |
| CMT24-02634 | Q017H-V045R-A068S-T077N- | 0.61 | 0.68 |
| | T078I-N096D-S108Q- | | |
| | G127S-A128P-G165Q-N184Q- | | |
| | A202V-G203E-N217S- | | |
| | S258P | | |
| CMT24-03043 | Q017H-A068S-T077N-T078I- | 0.88 | 0.99 |
| | N096D-G127S-A128P- | | |
| | S129Q-G165Q-N184Q-A202V- | | |
| | G203E-N217S-S258P- | | |
| | K264H | | |
| CMT24-03631 | A068S-T077N-T078I-S086D- | 0.75 | 0.91 |
| | S108Q-G127S-A128P- | | |
| | S129Q-G165Q-N184Q-A202V- | | |
| | G203E-N217S-S258P- | | |
| | K264H | | |
| CMT24-02816 | Q017H-V045R-A068S-T077N- | 0.60 | 0.66 |
| | T078I-N096D-S108Q- | | |
| | G127T-A128P-G165Q-N184Q- | | |
| | A202V-G203E-N217S- | | |
| | S258P | | |
| CMT24-02896 | Q017H-A068S-T077N-T078I- | 0.52 | 0.60 |
| | Y103F-S108Q-G127T- | | |
| | A128P-S129Q-G165Q-N184Q- | | |
| | A202V-G203N-N217S- | | |
| | S258P | | |
| CMT24-03662 | A068S-T077N-T078I-N096D- | 0.57 | 0.66 |
| | S108Q-T115L-G127T- | | |
| | A128P-G165Q-N184Q-A202V- | | |
| | G203E-N217S-S258P- | | |
| | K264H | | |
| CMT24-03394 | P009T-A068S-T077N-T078I- | 0.41 | 0.62 |
| | N096D-T115L-G127T- | | |
| | A128K-S129Q-G165Q-N184Q- | | |
| | A202V-N217S-S258P- | | |
| | K264H | | |
| CMT24-03311 | Q017H-A068S-T077N-T078I- | 0.41 | 0.53 |
| | Y103F-T115L-G127T- | | |
| | A128K-S129Q-G165Q-N184Q- | | |
| | A202V-N217S-S258P- | | |
| | K264H | | |
| CMT24-02892 | P009T-Q017H-T077N-T078I- | 0.60 | 0.87 |
| | N096D-Y103F-S108Q- | | |
| | G127S-A128P-G165Q-N184Q- | | |
| | A202V-G203N-N217S- | | |
| | S258P | | |
| CMT24-03388 | Q017H-A068S-T077N-T078I- | 0.52 | 0.72 |
| | N096D-T115L-G127T- | | |
| | A128K-S129Q-G165Q-N184Q- | | |
| | A202V-N217S-S258P- | | |
| | K264H | | |
| CMT24-03688 | A024Q-V045R-A068S-T077N- | 0.42 | 0.62 |
| | T078I-S108Q-G127S- | | |
| | A128K-S129Q-G165Q-N184Q- | | |
| | A202V-N217S-S258D- | | |
| | K264H | | |
| CMT24-03847 | A068S-T077N-T078I-S086D- | 0.71 | 1.02 |
| | Y103F-T115L-G127T- | | |
| | A128K-S129Q-G165Q-N184Q- | | |
| | A202V-G203E-N217S- | | |
| | S258P | | |
| CMT24-03262 | Q017H-A068S-T077N-T078I- | 0.45 | 0.57 |
| | N096D-S108Q-G127T- | | |
| | A128K-S129Q-G165Q-N184Q- | | |
| | A202V-N217S-S258P- | | |
| | K264H | | |
| CMT24-03697 | Q017H-A068S-T077N-T078I- | 0.49 | 0.47 |
| | S108Q-T115L-G117R- | | |
| | G127T-A128P-S129Q-G165Q- | | |
| | N184Q-A202V-N217S- | | |
| | S258D | | |
| CMT24-02709 | P009T-Q017H-V045R-T077N- | 0.68 | 0.88 |
| | T078I-N096D-S108Q- | | |
| | G127S-A128P-G165Q-N184Q- | | |
| | A202V-G203E-N217S- | | |
| | S258P | | |
| CMT24-03219 | Q017H-A068S-T077N-T078I- | 0.66 | 0.72 |
| | Y103F-T115L-G127T- | | |
| | A128K-S129Q-G165Q-N184Q- | | |
| | A202V-G203E-N217S- | | |
| | S258P | | |
| CMT24-03594 | Q017H-T077N-T078I-Y103F- | 0.47 | 0.58 |
| | T115L-G117R-G127S- | | |
| | A128P-S129Q-G165Q-N184Q- | | |
| | A202V-N217S-S258P- | | |
| | K264H | | |
| CMT24-02856 | P009T-Q017H-T077N-T078I- | 0.62 | 0.96 |
| | S108Q-G127S-A128P- | | |
| | S129Q-G165Q-N184Q-A202V- | | |
| | G203N-N217S-S258P- | | |
| | K264H | | |
| CMT24-03799 | A024Q-T077N-T078I-S086D- | 0.50 | 0.96 |
| | N096D-Y103F-S108Q- | | |
| | G117R-G127S-A128P-G165Q- | | |
| | N184Q-A202V-N217S- | | |
| | S258P | | |
| CMT24-03145 | P009T-V045R-T077N-T078I- | 0.45 | 0.49 |
| | N096D-S108Q-G127S- | | |
| | A128P-G165Q-N184Q-A202V- | | |
| | G203N-N217S-S258P- | | |
| | K264H | | |
| CMT24-03127 | Q017H-V045R-T077N-T078I- | 0.70 | 0.88 |
| | Y103F-G127S-A128P- | | |
| | S129Q-G165Q-N184Q-A202V- | | |
| | G203E-N217S-S258P- | | |
| | K264H | | |
| CMT24-03661 | P009T-T077N-T078I-N096D- | 0.53 | 0.69 |
| | S108Q-T115L-G127S- | | |
| | A128P-S129Q-G165Q-N184Q- | | |
| | A202V-N217S-S258P- | | |
| | K264H | | |
| CMT24-03357 | Q017H-A068S-T077N-T078I- | 0.96 | 0.95 |
| | Y103F-S108Q-G127T- | | |
| | A128K-S129Q-G165Q-N184Q- | | |
| | A202V-G203E-N217S- | | |
| | S258D | | |
| CMT24-03291 | P009T-A068S-T077N-T078I- | 0.51 | 0.50 |
| | Y103F-T115L-G127T- | | |
| | A128K-S129Q-G165Q-N184Q- | | |
| | A202V-G203E-N217S- | | |
| | S258D | | |
| CMT24-03320 | Q017H-A068S-T077N-T078I- | 0.51 | 0.72 |
| | S108Q-T115L-G127T- | | |
| | A128K-S129Q-G165Q-N184Q- | | |
| | A202V-G203E-N217S- | | |
| | S258D | | |
| CMT24-02922 | P009T-Q017H-V045R-T077N- | 0.59 | 0.95 |
| | T078I-N096D-G127T- | | |
| | A128P-G165Q-N184Q-A202V- | | |
| | G203N-N217S-S258P- | | |
| | K264H | | |
| CMT24-03071 | P009T-Q017H-T077N-T078I- | 0.43 | 0.90 |
| | N096D-Y103F-S108Q- | | |
| | G127T-A128P-S129Q-G165Q- | | |
| | N184Q-A202V-N217S- | | |
| | S258P | | |
| CMT24-03110 | P009T-Q017H-T077N-T078I- | 0.73 | 0.99 |
| | N096D-Y103F-S108Q- | | |
| | G127T-A128P-G165Q-N184Q- | | |
| | A202V-G203E-N217S- | | |
| | S258P | | |
| CMT24-03202 | P009T-A068S-T077N-T078I- | 0.42 | 0.65 |
| | N096D-T115L-G127T- | | |
| | A128K-S129Q-G165Q-N184Q- | | |
| | A202V-N217S-S258D- | | |
| | K264H | | |
| CMT24-02870 | Q017H-V045R-T077N-T078I- | 0.59 | 0.70 |
| | N096D-S108Q-G127T- | | |
| | A128P-S129Q-G165Q-N184Q- | | |
| | A202V-G203E-N217S- | | |
| | S258P | | |
| CMT24-03095 | P009T-Q017H-V045R-T077N- | 0.55 | 0.61 |
| | T078I-N096D-S108Q- | | |
| | G127T-A128P-G165Q-N184Q- | | |
| | A202V-G203N-N217S- | | |
| | S258P | | |
| CMT24-03131 | P009T-Q017H-V045R-T077N- | 0.51 | 0.69 |
| | T078I-S108Q-G127T- | | |
| | A128P-S129Q-G165Q-N184Q- | | |
| | A202V-N217S-S258P- | | |
| | K264H | | |
| CMT24-03788 | V045R-T077N-T078I-S086D- | 0.78 | 0.79 |
| | S108Q-T115L-G127S- | | |
| | A128K-S129Q-G165Q-N184Q- | | |
| | A202V-G203E-N217S- | | |
| | S258P | | |
| CMT24-02842 | Q017H-V045R-T077N-T078I- | 0.54 | 0.53 |
| | Y103F-G127T-A128P- | | |
| | S129Q-G165Q-N184Q-A202V- | | |
| | G203N-N217S-S258P- | | |
| | K264H | | |
| CMT24-02915 | Q017H-T077N-T078I-N096D- | 0.85 | 0.91 |
| | S108Q-G127T-A128P- | | |
| | S129Q-G165Q-N184Q-A202V- | | |
| | G203E-N217S-S258P- | | |
| | K264H | | |
| CMT24-03848 | T077N-T078I-S086D-S108Q- | 0.76 | 1.07 |
| | T115L-G117R-G127T- | | |
| | A128P-S129Q-G165Q-N184Q- | | |
| | A202V-G203E-N217S- | | |
| | S258P | | |
| CMT24-03216 | Q017H-A068S-T077N-T078I- | 0.51 | 0.62 |
| | S108Q-T115L-G127T- | | |
| | A128K-S129Q-G165Q-N184Q- | | |
| | A202V-N217S-S258D- | | |
| | K264H | | |
| CMT24-02940 | T077N-T078I-N096D-Y103F- | 0.59 | 0.76 |
| | S108Q-G127T-A128P- | | |
| | S129Q-G165Q-N184Q-A202V- | | |
| | G203E-N217S-S258P- | | |
| | K264H | | |
| CMT24-03090 | P009T-Q017H-V045R-T077N- | 0.46 | 0.77 |
| | T078I-Y103F-G127T- | | |
| | A128P-S129Q-G165Q-N184Q- | | |
| | A202V-N217S-S258P- | | |
| | K264H | | |
| CMT24-02980 | P009T-V045R-T077N-T078I- | 0.59 | 0.59 |
| | Y103F-G127T-A128P- | | |
| | S129Q-G165Q-N184Q-A202V- | | |
| | G203E-N217S-S258P- | | |
| | K264H | | |
| CMT24-03330 | P009T-Q017H-T077N-T078I- | 0.83 | 1.08 |
| | N096D-Y103F-G127T- | | |
| | A128K-S129Q-G165Q-N184Q- | | |
| | A202V-G203E-N217S- | | |
| | S258P | | |
| CMT24-03317 | P009T-V045R-T077N-T078I- | 0.51 | 0.53 |
| | N096D-Y103F-G127T- | | |
| | A128K-S129Q-G165Q-N184Q- | | |
| | A202V-G203E-N217S- | | |
| | S258P | | |
| CMT24-03481 | Q017H-T077N-T078I-N096D- | 0.41 | 0.69 |
| | Y103F-S108Q-G127T- | | |
| | A128K-S129Q-G165Q-N184Q- | | |
| | A202V-G203N-N217S- | | |
| | S258P | | |
| CMT24-03412 | Q017H-T077N-T078I-N096D- | 0.92 | 0.89 |
| | Y103F-S108Q-G127T- | | |
| | A128K-S129Q-G165Q-N184Q- | | |
| | A202V-G203E-N217S- | | |
| | S258P | | |
| CMT24-03270 | Q017H-V045R-T077N-T078I- | 0.62 | 0.49 |
| | S108Q-G127T-A128K- | | |
| | S129Q-G165Q-N184Q-A202V- | | |
| | G203E-N217S-S258P- | | |
| | K264H | | |
| CMT24-03525 | P009T-T077N-T078I-S086D- | 0.89 | 0.94 |
| | N096D-Y103F-S108Q- | | |
| | G127T-A128P-G165Q-N184Q- | | |
| | A202V-G203N-N217S- | | |
| | S258D | | |
| CMT24-03347 | P009T-T077N-T078I-Y103F- | 0.64 | 0.77 |
| | T115L-G127T-A128K- | | |
| | S129Q-G165Q-N184Q-A202V- | | |
| | G203E-N217S-S258P- | | |
| | K264H | | |
| CMT24-03822 | P009T-V045R-T077N-T078I- | 0.55 | 0.89 |
| | S086D-N096D-Y103F- | | |
| | G127T-A128K-S129Q-G165Q- | | |
| | N184Q-A202V-N217S- | | |
| | S258P | | |
| CMT24-03229 | P009T-Q017H-T077N-T078I- | 0.57 | 0.85 |
| | N096D-S108Q-G127T- | | |
| | A128K-S129Q-G165Q-N184Q- | | |
| | A202V-N217S-S258P- | | |
| | K264H | | |
| CMT24-03702 | Q017H-A024Q-T077N-T078I- | 0.70 | 0.91 |
| | S086D-Y103F-T115L- | | |
| | G127S-A128K-S129Q-G165Q- | | |
| | N184Q-A202V-N217S- | | |
| | S258D | | |
| CMT24-03218 | Q017H-V045R-T077N-T078I- | 0.44 | 0.60 |
| | N096D-Y103F-G127T- | | |
| | A128K-S129Q-G165Q-N184Q- | | |
| | A202V-N217S-S258D- | | |
| | K264H | | |
| CMT24-03139 | P009T-V045R-A068S-T077N- | 0.41 | 0.58 |
| | T078I-N096D-S108Q- | | |
| | G127S-A128P-S129Q-G165Q- | | |
| | N184Q-A202V-N217S- | | |
| | S258P-K264H | | |
| CMT24-02987 | Q017H-V045R-A068S-T077N- | 0.51 | 0.65 |
| | T078I-N096D-S108Q- | | |
| | G127S-A128P-G165Q-N184Q- | | |
| | A202V-G203N-N217S- | | |
| | S258P-K264H | | |
| CMT24-03655 | Q017H-A068S-T077N-T078I- | 0.50 | 0.86 |
| | S086D-N096D-S108Q- | | |
| | T115L-G117R-G127T-A128P- | | |
| | G165Q-N184Q-A202V- | | |
| | N217S-S258P | | |
| CMT24-03843 | A068S-T077N-T078I-S086D- | 0.46 | 0.75 |
| | N096D-Y103F-S108Q- | | |
| | T115L-G117R-G127T-A128P- | | |
| | G165Q-N184Q-A202V- | | |
| | N217S-S258P | | |
| CMT24-03522 | V045R-A068S-T077N-T078I- | 0.88 | 0.89 |
| | S086D-S108Q-G127T- | | |
| | A128P-S129Q-G165Q-N184Q- | | |
| | A202V-G203E-N217S- | | |
| | S258P-K264H | | |
| CMT24-03531 | V045R-A068S-T077N-T078I- | 0.62 | 0.86 |
| | S086D-N096D-S108Q- | | |
| | T115L-G127S-A128K-G165Q- | | |
| | N184Q-A202V-N217S- | | |
| | S258P-K264H | | |
| CMT24-03603 | A068S-T077N-T078I-S086D- | 0.75 | 0.79 |
| | S108Q-T115L-G117R- | | |
| | G127S-A128K-S129Q-G165Q- | | |
| | N184Q-A202V-G203E- | | |
| | N217S-S258D | | |
| CMT24-03179 | P009T-A068S-T077N-T078I- | 0.60 | 0.63 |
| | N096D-Y103F-T115L- | | |
| | G127T-A128K-S129Q-G165Q- | | |
| | N184Q-A202V-N217S- | | |
| | S258P-K264H | | |
| CMT24-03704 | A024Q-A068S-T077N-T078I- | 0.78 | 1.05 |
| | S086D-S108Q-T115L- | | |
| | G127T-A128K-S129Q-G165Q- | | |
| | N184Q-A202V-G203N- | | |
| | N217S-S258P | | |
| CMT24-03410 | Q017H-A068S-T077N-T078I- | 0.80 | 1.05 |
| | N096D-S108Q-T115L- | | |
| | G127T-A128K-S129Q-G165Q- | | |
| | N184Q-A202V-G203E- | | |
| | N217S-S258P | | |
| CMT24-03405 | A068S-T077N-T078I-N096D- | 0.54 | 0.62 |
| | Y103F-S108Q-T115L- | | |
| | G127T-A128K-S129Q-G165Q- | | |
| | N184Q-A202V-G203E- | | |
| | N217S-S258P | | |
| CMT24-03494 | P009T-V045R-T077N-T078I- | 0.84 | 1.14 |
| | S086D-S108Q-T115L- | | |
| | G127S-A128P-S129Q-G165Q- | | |
| | N184Q-A202V-G203E- | | |
| | N217S-S258P | | |
| CMT24-03191 | Q017H-A068S-T077N-T078I- | 0.63 | 0.75 |
| | N096D-Y103F-T115L- | | |
| | G127T-A128K-S129Q-G165Q- | | |
| | N184Q-A202V-N217S- | | |
| | S258P-K264H | | |
| CMT24-03574 | Q017H-V045R-A068S-T077N- | 0.56 | 0.82 |
| | T078I-S086D-Y103F- | | |
| | G127T-A128K-S129Q-G165Q- | | |
| | N184Q-A202V-N217S- | | |
| | S258D-K264H | | |
| CMT24-03777 | Q017H-A024Q-T077N-T078I- | 0.66 | 0.86 |
| | S086D-Y103F-S108Q- | | |
| | G127T-A128P-S129Q-G165Q- | | |
| | N184Q-A202V-G203N- | | |
| | N217S-S258P | | |
| CMT24-03725 | P009T-Q017H-T077N-T078I- | 0.80 | 0.91 |
| | Y103F-T115L-G117R- | | |
| | G127T-A128P-S129Q-G165Q- | | |
| | N184Q-A202V-G203E- | | |
| | N217S-S258P | | |
| CMT24-03877 | P009T-T077N-T078I-S086D- | 0.59 | 0.83 |
| | N096D-S108Q-T115L- | | |
| | G117R-G127T-A128P-S129Q- | | |
| | G165Q-N184Q-A202V- | | |
| | N217S-S258P | | |
| CMT24-03237 | A068S-T077N-T078I-N096D- | 0.46 | 0.56 |
| | Y103F-T115L-G127T- | | |
| | A128K-S129Q-G165Q-N184Q- | | |
| | A202V-G203E-N217S- | | |
| | S258D-K264H | | |
| CMT24-03485 | Q017H-V045R-T077N-T078I- | 0.73 | 1.02 |
| | S086D-S108Q-G127T- | | |
| | A128P-S129Q-G165Q-N184Q- | | |
| | A202V-G203E-N217S- | | |
| | S258D-K264H | | |
| CMT24-03296 | Q017H-V045R-T077N-T078I- | 0.70 | 0.63 |
| | N096D-T115L-G127T- | | |
| | A128K-S129Q-G165Q-N184Q- | | |
| | A202V-G203E-N217S- | | |
| | S258P-K264H | | |
| CMT24-03399 | T077N-T078I-Y103F-S108Q- | 0.41 | 0.44 |
| | T115L-G117R-G127T- | | |
| | A128K-S129Q-G165Q-N184Q- | | |
| | A202V-G203E-N217S- | | |
| | S258P-K264H | | |
| CMT24-03267 | P009T-Q017H-T077N-T078I- | 0.80 | 0.81 |
| | N096D-S108Q-T115L- | | |
| | G127T-A128K-S129Q-G165Q- | | |
| | N184Q-A202V-G203E- | | |
| | N217S-S258P | | |
| CMT24-03797 | P009T-Q017H-A024Q-T077N- | 0.92 | 1.00 |
| | T078I-Y103F-T115L- | | |
| | G127T-A128K-S129Q-G165Q- | | |
| | N184Q-A202V-G203E- | | |
| | N217S-S258P | | |
| CMT24-03425 | P009T-T077N-T078I-N096D- | 0.67 | 0.89 |
| | Y103F-S108Q-T115L- | | |
| | G127T-A128K-S129Q-G165Q- | | |
| | N184Q-A202V-G203E- | | |
| | N217S-S258P | | |
| CMT24-03652 | P009T-A024Q-T077N-T078I- | 0.79 | 1.00 |
| | S086D-Y103F-T115L- | | |
| | G127S-A128K-S129Q-G165Q- | | |
| | N184Q-A202V-G203N- | | |
| | N217S-S258D | | |
| CMT24-03195 | P009T-V045R-T077N-T078I- | 0.72 | 0.69 |
| | N096D-Y103F-G127T- | | |
| | A128K-S129Q-G165Q-N184Q- | | |
| | A202V-G203E-N217S- | | |
| | S258P-K264H | | |
| CMT24-03196 | P009T-Q017H-T077N-T078I- | 0.87 | 0.87 |
| | N096D-Y103F-T115L- | | |
| | G127T-A128K-S129Q-G165Q- | | |
| | N184Q-A202V-G203E- | | |
| | N217S-S258P | | |
| CMT24-03312 | Q017H-T077N-T078I-S108Q- | 0.66 | 0.69 |
| | T115L-G117R-G127T- | | |
| | A128K-S129Q-G165Q-N184Q- | | |
| | A202V-G203E-N217S- | | |
| | S258D-K264H | | |
| CMT24-03742 | Q017H-T077N-T078I-S086D- | 0.68 | 0.85 |
| | N096D-Y103F-S108Q- | | |
| | G117R-G127T-A128K-S129Q- | | |
| | G165Q-N184Q-A202V- | | |
| | N217S-S258D | | |
| CMT24-03517 | V045R-T077N-T078I-S086D- | 0.61 | 0.85 |
| | Y103F-S108Q-T115L- | | |
| | G127T-A128K-S129Q-G165Q- | | |
| | N184Q-A202V-N217S- | | |
| | S258D-K264H | | |
| CMT24-03184 | P009T-Q017H-V045R-T077N- | 0.96 | 0.75 |
| | T078I-Y103F-T115L- | | |
| | G127T-A128K-S129Q-G165Q- | | |
| | N184Q-A202V-G203E- | | |
| | N217S-S258D | | |
| CMT24-03860 | A024Q-V045R-A068S-T077N- | 0.57 | 0.79 |
| | T078I-Y103F-S108Q- | | |
| | T115L-G127S-A128P-S129Q- | | |
| | G165Q-N184Q-A202V- | | |
| | G203E-N217S-S258P | | |
| CMT24-03670 | P009T-V045R-A068S-T077N- | 0.68 | 0.92 |
| | T078I-S086D-N096D- | | |
| | S108Q-T115L-G127S-A128P- | | |
| | G165Q-N184Q-A202V- | | |
| | N217S-S258P-K264H | | |
| CMT24-03873 | Q017H-A024Q-V045R-A068S- | 0.78 | 1.02 |
| | T077N-T078I-S086D- | | |
| | Y103F-G127T-A128P-S129Q- | | |
| | G165Q-N184Q-A202V- | | |
| | G203E-N217S-S258P | | |
| CMT24-03653 | P009T-A068S-T077N-T078I- | 0.70 | 0.89 |
| | S086D-N096D-Y103F- | | |
| | T115L-G127S-A128K-S129Q- | | |
| | G165Q-N184Q-A202V- | | |
| | G203N-N217S-S258P | | |
| CMT24-03677 | Q017H-V045R-A068S-T077N- | 0.86 | 1.05 |
| | T078I-S086D-Y103F- | | |
| | S108Q-G127T-A128P-S129Q- | | |
| | G165Q-N184Q-A202V- | | |
| | G203E-N217S-S258P | | |
| CMT24-03497 | Q017H-A024Q-A068S-T077N- | 0.72 | 0.89 |
| | T078I-S108Q-T115L- | | |
| | G127T-A128P-S129Q-G165Q- | | |
| | N184Q-A202V-G203N- | | |
| | N217S-S258P-K264H | | |
| CMT24-03384 | Q017H-V045R-A068S-T077N- | 0.63 | 0.58 |
| | T078I-Y103F-T115L- | | |
| | G127T-A128K-S129Q-G165Q- | | |
| | N184Q-A202V-G203E- | | |
| | N217S-S258P-K264H | | |
| CMT24-03294 | P009T-A068S-T077N-T078I- | 0.71 | 0.74 |
| | N096D-Y103F-S108Q- | | |
| | T115L-G127T-A128K-S129Q- | | |
| | G165Q-N184Q-A202V- | | |
| | G203E-N217S-S258P | | |
| CMT24-03695 | P009T-V045R-A068S-T077N- | 0.82 | 0.86 |
| | T078I-S086D-Y103F- | | |
| | T115L-G127T-A128P-S129Q- | | |
| | G165Q-N184Q-A202V- | | |
| | G203N-N217S-S258D | | |
| CMT24-03592 | A024Q-A068S-T077N-T078I- | 0.82 | 0.85 |
| | N096D-Y103F-S108Q- | | |
| | T115L-G127T-A128K-G165Q- | | |
| | N184Q-A202V-G203E- | | |
| | N217S-S258P-K264H | | |
| CMT24-03566 | Q017H-A024Q-V045R-T077N- | 0.58 | 0.74 |
| | T078I-N096D-Y103F- | | |
| | T115L-G127S-A128P-S129Q- | | |
| | G165Q-N184Q-A202V- | | |
| | G203N-N217S-S258P | | |
| CMT24-03775 | Q017H-V045R-T077N-T078I- | 0.84 | 0.80 |
| | S086D-S108Q-T115L- | | |
| | G117R-G127T-A128P-S129Q- | | |
| | G165Q-N184Q-A202V- | | |
| | G203E-N217S-S258P | | |
| CMT24-03565 | Q017H-V045R-T077N-T078I- | 0.68 | 0.95 |
| | S086D-N096D-Y103F- | | |
| | S108Q-G127S-A128K-S129Q- | | |
| | G165Q-N184Q-A202V- | | |
| | N217S-S258P-K264H | | |
| CMT24-03226 | Q017H-A068S-T077N-T078I- | 0.77 | 0.84 |
| | N096D-S108Q-T115L- | | |
| | G127T-A128K-S129Q-G165Q- | | |
| | N184Q-A202V-G203E- | | |
| | N217S-S258D-K264H | | |
| CMT24-03206 | P009T-Q017H-V045R-T077N- | 0.85 | 0.78 |
| | T078I-N096D-Y103F- | | |
| | S108Q-G127T-A128K-S129Q- | | |
| | G165Q-N184Q-A202V- | | |
| | G203E-N217S-S258P | | |
| CMT24-03712 | P009T-V045R-T077N-T078I- | 0.79 | 0.83 |
| | S086D-Y103F-T115L- | | |
| | G117R-G127T-A128P-S129Q- | | |
| | G165Q-N184Q-A202V- | | |
| | G203N-N217S-S258D | | |
| CMT24-03595 | P009T-Q017H-V045R-A068S- | 0.72 | 0.83 |
| | T077N-T078I-N096D- | | |
| | Y103F-S108Q-T115L-G127S- | | |
| | A128K-G165Q-N184Q- | | |
| | A202V-G203E-N217S-S258P | | |
| CMT24-03817 | A024Q-A068S-T077N-T078I- | 0.78 | 0.91 |
| | S086D-Y103F-S108Q- | | |
| | T115L-G117R-G127T-A128K- | | |
| | S129Q-G165Q-N184Q- | | |
| | A202V-G203E-N217S-S258P | | |
| CMT24-03639 | P009T-Q017H-A068S-T077N- | 0.74 | 1.06 |
| | T078I-Y103F-S108Q- | | |
| | T115L-G127T-A128K-S129Q- | | |
| | G165Q-N184Q-A202V- | | |
| | G203N-N217S-S258D-K264H | | |
| CMT24-03716 | A024Q-V045R-A068S-T077N- | 0.95 | 0.88 |
| | T078I-S086D-N096D- | | |
| | Y103F-S108Q-T115L-G127S- | | |
| | A128K-S129Q-G165Q- | | |
| | N184Q-A202V-G203E-N217S- | | |
| | S258P | | |

TABLE 5
Stability evaluation of AprL variants reported as fraction of residual activity
when tested under conditions (temperature and detergent) described.

| | | 10% | |
|---|---|---|---|
| | | Test | 20% |
| | | HDLA | CNS |
| Enzyme Name | Substitutions vs AprL | 72° C. | 70° C. |
| AprL-WT | none | 0.00 | 0.00 |
| CMT24 | A068S-T077N-T078I-G127S-A128P-G165Q-N184Q- | 0.00 | 0.06 |
| | A202V-N217S-S258P | | |
| CMT24-05788 | Q017H-A024Q-V045R-T077N-T078I-N096D-S100N- | 0.21 | 0.40 |
| | G127S-A128P-S155E-G165Q-A202V-G203E-N217S | | |
| CMT24-06206 | Q017H-A024Q-V045R-T077N-T078I-N096D-Y103I- | 0.22 | 0.44 |
| | G127S-A128P-G165Q-S181Q-A202V-G203E-N217S | | |
| CMT24-06498 | P009T-Q017H-T077N-T078I-V087A-N096D-G127T- | 0.28 | 0.61 |
| | A128K-T161Q-G165Q-S181Q-A202V-G203E-S258D | | |
| CMT24-05783 | Q017H-A024Q-V045R-T077N-N096D-Y103F-A128P- | 0.33 | 0.33 |
| | T161Q-G165Q-S181Q-A202V-G203E-N217S-K264H | | |
| CMT24-05810 | Q017H-A024Q-V045R-T077N-T078I-N096D-Y103I- | 0.35 | 0.48 |
| | G127S-A128P-G165Q-A202V-G203E-N217S-K264H | | |
| CMT24-05765 | A024Q-V087A-N096D-Y103F-G127T-A128K-S129Q- | 0.39 | 0.48 |
| | G165Q-S181Q-A202V-G203E-N217S-F260W-K264H | | |
| CMT24-06760 | P009T-Q017H-T077N-T078I-V087A-N096D-T115L- | 0.27 | 0.52 |
| | G127T-A128K-G165Q-S181Q-A202V-G203E-S258D- | | |
| | F260W | | |
| CMT24-06126 | Q017H-A024Q-V045R-T077N-T078I-N096D-S100N- | 0.34 | 0.48 |
| | Y103I-A128P-T161Q-G165Q-A202V-G203E-N217S- | | |
| | K264H | | |
| CMT24-06294 | Q017H-V045R-T077N-T078I-S086D-N096D-Y103I- | 0.34 | 0.60 |
| | G127T-A128K-S129Q-G165Q-A202V-G203E-S258P- | | |
| | K264H | | |
| CMT24-06749 | P009T-Q017H-T077N-T078I-N096D-Y103I-G127T- | 0.33 | 0.41 |
| | A128K-S129Q-T161Q-G165Q-S181Q-A202V-G203E- | | |
| | F260W-K264H | | |
| CMT24-06543 | Q017H-A024Q-V045R-T077N-T078I-N096D-G127T- | 0.33 | 0.38 |
| | A128K-S129Q-S155E-G165Q-N184Q-A202V-G203N- | | |
| | N217S-K264H | | |
| CMT24-05738 | A024Q-T077N-N096D-Y103F-S108Q-G127T-A128K- | 0.44 | 0.51 |
| | S129Q-G165Q-S181Q-A202V-G203E-N217S-M221Q- | | |
| | F260W-K264H | | |
| CMT24-05761 | A024Q-T077N-V087A-N096D-Y103F-T115L-G127T- | 0.48 | 0.45 |
| | A128K-S129Q-G165Q-S181Q-G203E-N217S-M221Q- | | |
| | F260W-K264H | | |
| CMT24-06437 | P009T-Q017H-T077N-T078I-V087A-N096D-Y103I- | 0.63 | 0.71 |
| | G127T-A128K-S129Q-G165Q-S181Q-A202V-G203E- | | |
| | S258P-K264H | | |
| CMT24-06230 | Q017H-A024Q-V045R-T077N-T078I-N096D-Y103F- | 0.30 | 0.50 |
| | S108Q-G127S-A128P-S155E-T161Q-G165Q-S181Q- | | |
| | A202V-G203E-N217S | | |
| CMT24-06273 | Q017H-V045R-A068S-T077N-T078I-S086D-N096D- | 0.36 | 0.51 |
| | Y103I-G127T-A128K-S129Q-S155E-G165Q-A202V- | | |
| | G203N-S258P-K264H | | |
| CMT24-05823 | Q017H-V045R-T077N-V087A-N096D-Y103F-T115L- | 0.36 | 0.41 |
| | G127T-A128K-S129Q-G165Q-S181Q-A202V-G203E- | | |
| | N217S-M221Q-K264H | | |
| CMT24-06400 | Q017H-A024Q-V045R-A068S-T077N-T078I-S086D- | 0.40 | 0.58 |
| | N096D-Y103I-G127T-A128K-S129Q-G165Q-A202V- | | |
| | G203E-S258P-K264H | | |
| CMT24-07565 | P009T-Q017H-A024Q-T077N-T078I-N096D-Y103F- | 0.43 | 0.57 |
| | G127T-S129Q-G165Q-S181Q-N184Q-A202V-G203E- | | |
| | S258D-F260W-K264H | | |
| CMT24-05806 | Q017H-A024Q-V045R-T077N-T078I-N096D-S100N- | 0.45 | 0.62 |
| | Y103F-S108Q-A128P-T161Q-G165Q-S181Q-A202V- | | |
| | G203E-N217S-K264H | | |
| CMT24-06178 | T077N-T078I-V087A-N096D-Y103F-S108Q-G127T- | 0.65 | 0.57 |
| | A128K-S129Q-G165Q-S181Q-A202V-G203E-N217S- | | |
| | M221Q-F260W-K264H | | |
| CMT24-06186 | P009T-Q017H-A024Q-T077N-T078I-N096D-Y103F- | 0.74 | 0.63 |
| | G127T-A128K-S129Q-G165Q-S181Q-A202V-G203E- | | |
| | N217S-F260W-K264H | | |
| CMT24-05950 | Q017H-V045R-A068S-T077N-T078I-S086D-N096D- | 0.43 | 0.72 |
| | Y103I-S108Q-G127T-A128K-S129Q-G165Q-S181Q- | | |
| | A202V-G203E-S258P-K264H | | |
| CMT24-06182 | T077N-T078I-V087A-N096D-Y103F-S108Q-T115L- | 0.46 | 0.64 |
| | G127T-A128K-S129Q-G165Q-S181Q-N184Q-A202V- | | |
| | G203E-N217S-M221Q-K264H | | |
| CMT24-06890 | P009T-Q017H-V045R-T077N-T078I-S086E-N096D- | 0.47 | 0.56 |
| | S108Q-G127T-A128K-S129Q-S155E-G165Q-S181Q- | | |
| | N184Q-A202V-N217S-K264H | | |
| CMT24-05775 | A024Q-V045R-V087A-N096D-Y103F-S108Q-T115L- | 0.52 | 0.56 |
| | G127T-A128K-S129Q-G165Q-S181Q-A202V-G203E- | | |
| | N217S-M221Q-F260W-K264H | | |
| CMT24-05753 | P009T-Q017H-V045R-T077N-T078I-S086D-N096D- | 0.53 | 0.75 |
| | S100N-Y103F-G127T-A128K-S129Q-G165Q-A202V- | | |
| | G203E-S258P-F260W-K264H | | |
| CMT24-07308 | P009T-Q017H-A024Q-T077N-T078I-N096D-S100E- | 0.60 | 0.80 |
| | Y103F-G127T-A128K-S129Q-G165Q-S181Q-A202V- | | |
| | G203E-N217S-F260W-K264H | | |
| CMT24-05931 | P009T-Q017H-A024Q-T077N-T078I-N096D-S100N- | 0.61 | 0.75 |
| | Y103F-S108Q-G127T-A128K-S129Q-G165Q-S181Q- | | |
| | A202V-G203E-N217S-F260W | | |
| CMT24-06552 | P009T-Q017H-A024Q-V045R-T077N-T078I-N096D- | 0.61 | 0.78 |
| | S108Q-G127T-A128K-S129Q-S155E-G165Q-S181Q- | | |
| | A202V-G203E-N217S-K264H | | |
| CMT24-06611 | P009T-Q017H-V045R-T077N-N096D-Y103F-G127T- | 0.63 | 0.67 |
| | A128K-S129Q-S155E-G165Q-S181Q-N184Q-A202V- | | |
| | G203E-M221Q-S258P-K264H | | |
| CMT24-06213 | P009T-Q017H-A024Q-T077N-T078I-N096D-S100N- | 0.74 | 0.89 |
| | Y103F-G127T-A128K-S129Q-G165Q-S181Q-A202V- | | |
| | G203E-N217S-F260W-K264H | | |
| CMT24-06107 | A024Q-V045R-T077N-T078I-V087A-N096D-Y103F- | 0.75 | 0.78 |
| | T115L-G127T-A128K-S129Q-G165Q-S181Q-A202V- | | |
| | G203E-N217S-M221Q-K264H | | |
| CMT24-07670 | P009T-Q017H-V045R-T077N-T078I-N096D-Y103F- | 0.42 | 0.65 |
| | G127T-A128K-S129Q-G165Q-S181E-N184Q-A202V- | | |
| | G203E-M221Q-S258P-F260W-K264H | | |
| CMT24-07512 | P009T-Q017H-A024Q-V045R-T077N-T078I-N096D- | 0.43 | 0.59 |
| | S100N-Y103F-S108Q-G127T-A128K-S129Q-G165Q- | | |
| | S181Q-A202V-G203E-N217S-F260W | | |
| CMT24-06160 | Q017H-A024Q-V045R-T077N-V087A-N096D-Y103F- | 0.45 | 0.62 |
| | S108Q-G127T-A128K-S129Q-G165Q-S181Q-N184Q- | | |
| | A202V-G203E-N217S-F260W-K264H | | |
| CMT24-07342 | P009T-Q017H-A024Q-V045R-T077N-T078I-N096D- | 0.47 | 0.63 |
| | S100N-Y103F-T115L-G127T-A128K-S129Q-G165Q- | | |
| | S181Q-A202V-G203E-N217S-F260W | | |
| CMT24-05968 | Q017H-A024Q-V045R-T077N-T078I-S086D-N096D- | 0.51 | 0.67 |
| | Y103I-G127T-A128K-S129Q-S155E-G165Q-S181Q- | | |
| | A202V-G203N-S258P-F260W-K264H | | |
| CMT24-07417 | P009T-Q017H-A024Q-V045R-T077N-T078I-N096D- | 0.53 | 0.53 |
| | S100E-Y103F-S108Q-G127T-A128K-S129Q-G165Q- | | |
| | S181Q-A202V-G203E-N217S-K264H | | |
| CMT24-05893 | P009T-Q017H-A024Q-T077N-T078I-N096D-S100E- | 0.55 | 0.40 |
| | Y103F-S108Q-T115L-G127T-A128K-S129Q-G165Q- | | |
| | S181Q-A202V-G203E-N217S-F260W | | |
| CMT24-07552 | P009T-Q017H-A024Q-V045R-T077N-T078I-N096D- | 0.66 | 0.63 |
| | S100N-Y103F-G127T-A128K-S129Q-G165Q-S181Q- | | |
| | A202V-G203E-N217S-F260W-K264H | | |
| CMT24-06154 | A024Q-T077N-T078I-V087A-N096D-Y103F-S108Q- | 0.78 | 0.83 |
| | G127T-A128K-S129Q-G165Q-S181Q-N184Q-A202V- | | |
| | G203E-N217S-M221Q-F260W-K264H | | |
| CMT24-07315 | P009T-Q017H-V045R-T077N-T078I-S086D-N096D- | 0.40 | 0.49 |
| | S100N-Y103F-S108Q-G127T-A128K-S129Q-G165Q- | | |
| | S181E-A202V-G203E-S258P-F260W-K264H | | |
| CMT24-05972 | Q017H-A024Q-V045R-T077N-T078I-S086D-N096D- | 0.44 | 0.64 |
| | Y103I-T115L-G127T-A128K-S129Q-S155E-T161Q- | | |
| | G165Q-S181Q-A202V-S258P-F260W-K264H | | |
| CMT24-06007 | Q017H-V045R-A068S-T077N-T078I-S086D-N096D- | 0.52 | 0.71 |
| | Y103I-G127T-A128K-S129Q-S155E-T161Q-G165Q- | | |
| | N184Q-A202V-G203N-S258P-F260W-K264H | | |
| CMT24-07542 | Q017H-A024Q-V045R-T077N-T078I-V087A-N096D- | 0.53 | 0.64 |
| | Y103F-T115L-G127T-A128K-S129Q-G165Q-S181Q- | | |
| | N184Q-G203E-N217S-M221Q-F260W-K264H | | |
| CMT24-06201 | P009T-Q017H-A024Q-V045R-T077N-T078I-N096D- | 0.61 | 0.71 |
| | S100N-Y103F-S108Q-G127T-A128K-S129Q-G165Q- | | |
| | S181Q-A202V-G203E-N217S-F260W-K264H | | |
| CMT24-07569 | Q017H-A024Q-V045R-T077N-T078I-S086D-N096D- | 0.67 | 0.60 |
| | Y103I-G127T-A128K-S129Q-S155E-T161Q-G165Q- | | |
| | S181Q-A202V-G203E-S258P-F260W-K264H | | |
| CMT24-07481 | Q017H-A024Q-V045R-T077N-T078I-S086D-N096D- | 0.68 | 0.84 |
| | Y103I-G127T-A128K-S129Q-S155E-T161Q-G165Q- | | |
| | N184Q-A202V-G203E-S258P-F260W-K264H | | |
| CMT24-05722 | Q017H-A024Q-T077N-V087A-N096D-Y103F-S108Q- | 0.75 | 0.78 |
| | T115L-G127T-A128K-S129Q-G165Q-S181Q-N184Q- | | |
| | A202V-G203E-N217S-M221Q-F260W-K264H | | |
| CMT24-07523 | Q017H-T077N-T078I-V087A-N096D-Y103F-S108Q- | 0.78 | 0.84 |
| | T115L-G127T-A128K-S129Q-G165Q-S181Q-N184Q- | | |
| | A202V-G203E-N217S-M221Q-F260W-K264H | | |
| CMT24-07646 | P009T-Q017H-V045R-A068S-T077N-T078I-N096D- | 0.41 | 0.62 |
| | Y103F-G127T-A128K-S129Q-S155E-G165Q-S181Q- | | |
| | N184Q-A202V-G203E-M221Q-S258P-F260W-K264H | | |
| CMT24-07492 | Q017H-A024Q-V045R-A068S-T077N-T078I-S086D- | 0.41 | 0.63 |
| | N096D-Y103I-T115L-G127T-A128K-S129Q-S155E- | | |
| | G165Q-S181Q-A202V-G203E-S258P-F260W-K264H | | |
| CMT24-07592 | Q017H-A024Q-V045R-A068S-T077N-T078I-S086D- | 0.45 | 0.75 |
| | N096D-Y103I-G127T-A128K-S129Q-S155E-G165Q- | | |
| | S181Q-N184Q-A202V-G203N-S258P-F260W-K264H | | |
| CMT24-07661 | P009T-Q017H-V045R-T077N-T078I-N096D-Y103F- | 0.52 | 0.65 |
| | S108Q-T115L-G127T-A128K-S129Q-G165Q-S181Q- | | |
| | N184Q-A202V-G203E-M221Q-S258P-F260W-K264H | | |
| CMT24-07557 | Q017H-A024Q-V045R-T077N-T078I-S086D-N096D- | 0.54 | 0.61 |
| | Y103I-S108Q-T115L-G127T-A128K-S129Q-G165Q- | | |
| | S181Q-N184Q-A202V-G203E-S258P-F260W-K264H | | |
| CMT24-07634 | Q017H-A024Q-V045R-T077N-T078I-S086D-N096D- | 0.54 | 0.71 |
| | Y103I-T115L-G127T-A128K-S129Q-S155E-T161Q- | | |
| | G165Q-N184Q-A202V-G203E-S258P-F260W-K264H | | |
| CMT24-07609 | Q017H-A024Q-V045R-A068S-T077N-T078I-S086D- | 0.55 | 0.64 |
| | N096D-Y103I-G127T-A128K-S129Q-S155E-T161Q- | | |
| | G165Q-S181Q-A202V-G203E-S258P-F260W-K264H | | |
| CMT24-07507 | Q017H-A024Q-V045R-T077N-T078I-S086D-N096D- | 0.58 | 0.66 |
| | Y103F-S108Q-T115L-G127T-A128K-S129Q-S155E- | | |
| | G165Q-N184Q-A202V-G203E-S258P-F260W-K264H | | |
| CMT24-07455 | Q017H-A024Q-V045R-T077N-T078I-S086D-N096D- | 0.59 | 0.82 |
| | Y103I-T115L-G127T-A128K-S129Q-T161Q-G165Q- | | |
| | S181Q-N184Q-A202V-G203E-S258P-F260W-K264H | | |
| CMT24-07488 | Q017H-A024Q-V045R-T077N-T078I-S086D-N096D- | 0.60 | 0.74 |
| | Y103I-T115L-G127T-A128K-S129Q-S155E-T161Q- | | |
| | G165Q-S181Q-A202V-G203N-S258P-F260W-K264H | | |
| CMT24-07482 | Q017H-A024Q-V045R-T077N-T078I-S086D-N096D- | 0.66 | 0.93 |
| | Y103I-S108Q-G127T-A128K-S129Q-S155E-T161Q- | | |
| | G165Q-N184Q-A202V-G203E-S258P-F260W-K264H | | |
| CMT24-07501 | Q017H-A024Q-V045R-T077N-T078I-S086D-N096D- | 0.74 | 0.77 |
| | Y103I-G127T-A128K-S129Q-S155E-T161Q-G165Q- | | |
| | S181Q-N184Q-A202V-G203E-S258P-F260W-K264H | | |
| CMT24-07648 | P009T-Q017H-V045R-A068S-T077N-N096D-Y103F- | 0.42 | 0.42 |
| | S108Q-T115L-G127T-A128K-S129Q-S155E-G165Q- | | |
| | S181Q-N184Q-A202V-G203E-M221Q-S258P-F260W- | | |
| | K264H | | |
| CMT24-07631 | P009T-Q017H-V045R-A068S-T077N-T078I-N096D- | 0.46 | 0.70 |
| | Y103F-S108Q-T115L-G127T-A128K-S129Q-S155E- | | |
| | G165Q-N184Q-A202V-G203E-M221Q-S258P-F260W- | | |
| | K264H | | |
| CMT24-07587 | Q017H-A024Q-V045R-A068S-T077N-T078I-S086D- | 0.57 | 0.64 |
| | N096D-Y103I-S108Q-G127T-A128K-S129Q-S155E- | | |
| | T161Q-G165Q-S181Q-A202V-G203N-S258P-F260W- | | |
| | K264H | | |
| CMT24-07672 | P009T-Q017H-V045R-A068S-T077N-T078I-N096D- | 0.74 | 0.89 |
| | Y103F-T115L-G127T-A128K-S129Q-S155E-G165Q- | | |
| | S181E-N184Q-A202V-G203E-M221Q-S258P-F260W- | | |
| | K264H | | |
| CMT24-07504 | Q017H-A024Q-V045R-T077N-T078I-S086D-N096D- | 0.79 | 0.87 |
| | Y103I-S108Q-G127T-A128K-S129Q-S155E-T161Q- | | |
| | G165Q-S181Q-N184Q-A202V-G203E-S258P-F260W- | | |
| | K264H | | |
| CMT24-07491 | Q017H-A024Q-V045R-A068S-T077N-T078I-S086D- | 0.56 | 0.68 |
| | N096D-Y103I-S108Q-G127T-A128K-S129Q-S155E- | | |
| | T161Q-G165Q-S181Q-N184Q-A202V-G203N-S258P- | | |
| | F260W-K264H | | |
| CMT24-07497 | Q017H-A024Q-V045R-A068S-T077N-T078I-S086D- | 0.64 | 0.85 |
| | N096D-Y103I-S108Q-T115L-G127T-A128K-S129Q- | | |
| | S155E-T161Q-G165Q-N184Q-A202V-G203E-S258P- | | |
| | F260W-K264H | | |

Tables 6, 7, and 8 show results for enzyme samples generated from expression in B. licheniformis host using the method described in Example 1, obtained from shake flask cultures in clarified supernatant, and assayed for stability in detergent as described in Example 2.

TABLE 6
Stability evaluation of AprL variants reported as fraction of residual activity
when tested under conditions (temperature and detergent) described.

| | | 10% PNB | 20% CNS |
|---|---|---|---|
| Enzyme Name | Substitutions vs AprL | 69° C. | 69° C. |
| AprL-WT | none | 0.00 | 0.00 |
| CMT24 | A068S-T077N-T078I-G127S-A128P-G165Q- | 0.23 | 0.06 |
| | N184Q-A202V-N217S-S258P | | |
| CMT24-05500 | Q017H-T077N-T078I-S086D-N096D-G117R- | 0.53 | 0.65 |
| | G127S-A128P-G165Q-N184Q-A202V-N217S- | | |
| | S258P | | |
| CMT24-05504 | Q017H-V045R-T077N-T078I-S086D-N096D- | 0.52 | 0.61 |
| | G127S-A128P-G165Q-N184Q-A202V-N217S- | | |
| | S258P | | |
| CMT24-05511 | Q017H-V045R-T077N-T078I-N096D-G117R- | 0.65 | 0.43 |
| | G127S-A128P-G165Q-N184Q-A202V-G203E- | | |
| | N217S-S258D | | |
| CMT24-05516 | Q017H-V045R-T077N-T078I-N096D-G117R- | 0.64 | 0.48 |
| | G127T-A128P-G165Q-N184Q-A202V-G203E- | | |
| | N217S-S258D | | |
| CMT24-05507 | Q017H-V045R-T077N-T078I-S086D-N096D- | 0.84 | 0.82 |
| | G117R-G127S-A128P-G165Q-N184Q-A202V- | | |
| | G203E-N217S-S258P | | |
| CMT24-05508 | Q017H-V045R-T077N-T078I-S086D-N096D- | 0.73 | 0.74 |
| | G117R-G127S-A128P-G165Q-N184Q-A202V- | | |
| | N217S-S258P-K264H | | |
| CMT24-05509 | Q017H-V045R-T077N-T078I-N096D-G117R- | 0.67 | 0.52 |
| | G127S-A128P-G165Q-N184Q-A202V-G203E- | | |
| | N217S-S258P-K264H | | |
| CMT24-05512 | Q017H-V045R-T077N-T078I-S086D-N096D- | 0.86 | 0.81 |
| | G117R-G127T-A128P-G165Q-N184Q-A202V- | | |
| | G203E-N217S-S258P | | |
| CMT24-05513 | Q017H-V045R-T077N-T078I-S086D-N096D- | 0.78 | 0.74 |
| | G117R-G127T-A128P-G165Q-N184Q-A202V- | | |
| | N217S-S258P-K264H | | |
| CMT24-05514 | Q017H-V045R-T077N-T078I-N096D-G117R- | 0.67 | 0.52 |
| | G127T-A128P-G165Q-N184Q-A202V-G203E- | | |
| | N217S-S258P-K264H | | |
| CMT24-05519 | P009T-Q017H-T077N-T078I-N096D-T115L- | 0.60 | 0.61 |
| | G127S-A128P-S129Q-G165Q-N184Q-A202V- | | |
| | N217S-S258P-F260W | | |
| CMT24-05520 | P009T-Q017H-T077N-T078I-N096D-S108Q- | 0.62 | 0.60 |
| | G127S-A128P-S129Q-G165Q-N184Q-A202V- | | |
| | N217S-S258P-F260W | | |
| CMT24-05517 | P009T-Q017H-T077N-T078I-N096D-S108Q- | 0.60 | 0.61 |
| | T115L-G127T-A128P-S129Q-G165Q-N184Q- | | |
| | A202V-N217S-S258P-F260W | | |
| CMT24-05518 | P009T-Q017H-T077N-T078I-N096D-S108Q- | 0.59 | 0.62 |
| | T115L-G127S-A128P-S129Q-G165Q-N184Q- | | |
| | A202V-N217S-S258P-F260W | | |
| CMT24-05482 | P009T-Q017H-A024Q-V045R-T077N-T078I- | 0.90 | 0.88 |
| | N096D-S108Q-G127S-A128P-T161Q-G165Q- | | |
| | S181Q-N184Q-A202V-G203E-N217S-S258P | | |
| CMT24-05484 | P009T-Q017H-A024Q-V045R-T077N-T078I- | 0.87 | 0.85 |
| | N096D-S108Q-G127T-A128P-T161Q-G165Q- | | |
| | S181Q-N184Q-A202V-G203E-N217S-S258P | | |
| CMT24-05486 | P009T-Q017H-A024Q-T077N-T078I-N096D- | 0.91 | 0.88 |
| | S108Q-G117R-G127S-A128P-T161Q-G165Q- | | |
| | S181Q-N184Q-A202V-G203E-N217S-S258P | | |
| CMT24-05488 | P009T-Q017H-A024Q-T077N-T078I-N096D- | 0.90 | 0.89 |
| | S108Q-G117R-G127T-A128P-T161Q-G165Q- | | |
| | S181Q-N184Q-A202V-G203E-N217S-S258P | | |

TABLE 7
Stability evaluation of AprL variants reported as fraction of residual activity
when tested under conditions (temperature and detergent) described.

| | | 10% | 20% |
|---|---|---|---|
| | | PNB | CNS |
| Enzyme Name | Substitutions vs AprL | 75° C. | 72° C. |
| AprL-WT | none | 0.0 | 0.0 |
| CMT24 | A068S-T077N-T078I-G127S-A128P-G165Q-N184Q- | 0.0 | 0.0 |
| | A202V-N217S-S258P | | |
| CMT24-05582 | P009T-Q017H-V045R-T077N-T078I-N096D-Y103F- | 0.13 | 0.25 |
| | G127T-A128K-S129Q-S155E-G165Q-N184Q-A202V- | | |
| | G203E-N217S-S258P | | |
| CMT24-05547 | P009T-Q017H-V045R-A068S-T077N-T078I-N096D- | 0.19 | 0.26 |
| | S108Q-T115L-G127T-A128K-S129Q-G165Q-N184Q- | | |
| | A202V-G203E-N217S-S258P-K264H | | |
| CMT24-05572 | P009T-Q017H-V045R-A068S-T077N-T078I-N096D- | 0.20 | 0.31 |
| | S108Q-G127T-A128K-S129Q-G165Q-N184Q-A202V- | | |
| | G203E-N217S-S258P-K264H | | |
| CMT24-05535 | P009T-Q017H-V045R-T077N-T078I-N096D-Y103F- | 0.21 | 0.31 |
| | G127T-A128K-S129Q-G165Q-N184Q-A202V-G203E- | | |
| | N217S-S258P-K264H | | |
| CMT24-05573 | P009T-Q017H-V045R-A068S-T077N-T078I-N096D- | 0.22 | 0.36 |
| | S108Q-T115L-G127S-A128K-S129Q-G165Q-N184Q- | | |
| | A202V-G203E-N217S-S258P-K264H | | |
| CMT24-05541 | P009T-Q017H-V045R-T077N-T078I-N096D-Y103F- | 0.23 | 0.29 |
| | G127T-A128K-S129Q-T161Q-G165Q-S181Q-N184Q- | | |
| | A202V-G203E-N217S-S258D | | |
| CMT24-05571 | P009T-Q017H-V045R-A068S-T077N-T078I-N096D- | 0.23 | 0.29 |
| | T115L-G127T-A128K-S129Q-G165Q-N184Q-A202V- | | |
| | G203E-N217S-S258P-K264H | | |
| CMT24-05553 | P009T-Q017H-V045R-T077N-T078I-N096D-S108Q- | 0.24 | 0.32 |
| | T115L-G127T-A128K-S129Q-G165Q-N184Q-A202V- | | |
| | G203E-N217S-S258P-F260W-K264H | | |
| CMT24-05551 | P009T-Q017H-V045R-T077N-T078I-N096D-S108Q- | 0.25 | 0.33 |
| | T115L-G127T-A128K-S129Q-T161Q-G165Q-S181Q- | | |
| | N184Q-A202V-G203E-N217S-S258P-K264H | | |
| CMT24-05564 | P009T-Q017H-T077N-T078I-Y103F-G127T-A128K- | 0.25 | 0.30 |
| | S129Q-G165Q-N184Q-A202V-G203E-N217S-S258P | | |
| CMT24-05552 | P009T-Q017H-V045R-A068S-T077N-T078I-N096D- | 0.26 | 0.39 |
| | S108Q-T115L-G127T-A128K-S129Q-T161Q-G165Q- | | |
| | S181Q-N184Q-A202V-G203E-N217S-S258P-F260W- | | |
| | K264H | | |
| CMT24-05545 | P009T-Q017H-V045R-T077N-T078I-N096D-Y103F- | 0.27 | 0.41 |
| | G127T-A128K-S129Q-G165Q-N184Q-A202V-G203E- | | |
| | N217S-S258P-F260W-K264H | | |
| CMT24-05548 | P009T-Q017H-V045R-A068S-T077N-T078I-N096D- | 0.28 | 0.39 |
| | S108Q-T115L-G127T-A128K-S129Q-T161Q-G165Q- | | |
| | S181Q-N184Q-A202V-G203E-N217S-S258P-K264H | | |
| CMT24-05543 | P009T-Q017H-V045R-T077N-T078I-N096D-Y103F- | 0.29 | 0.36 |
| | G127T-A128K-S129Q-T161Q-G165Q-S181Q-N184Q- | | |
| | A202V-G203E-N217S-S258D-F260W | | |
| CMT24-05570 | P009T-Q017H-V045R-A068S-T077N-T078I-S108Q- | 0.30 | 0.25 |
| | T115L-G127T-A128K-S129Q-G165Q-N184Q-A202V- | | |
| | G203E-N217S-S258P-K264H | | |
| CMT24-05544 | P009T-Q017H-V045R-T077N-T078I-N096D-Y103F- | 0.31 | 0.36 |
| | G127T-A128K-S129Q-T161Q-G165Q-S181Q-N184Q- | | |
| | A202V-G203E-N217S-S258P-K264H | | |
| CMT24-05533 | P009T-Q017H-T077N-T078I-N096D-Y103F-G127T- | 0.31 | 0.45 |
| | A128K-S129Q-T161Q-G165Q-S181Q-N184Q-A202V- | | |
| | G203E-N217S-S258P | | |
| CMT24-05566 | P009T-Q017H-T077N-T078I-N096D-Y103F-G127S- | 0.31 | 0.40 |
| | A128K-S129Q-G165Q-N184Q-A202V-G203E-N217S- | | |
| | S258P | | |
| CMT24-05554 | P009T-Q017H-V045R-T077N-T078I-N096D-S108Q- | 0.33 | 0.46 |
| | T115L-G127T-A128K-S129Q-T161Q-G165Q-S181Q- | | |
| | N184Q-A202V-G203E-N217S-S258P-F260W-K264H | | |
| CMT24-05536 | P009T-Q017H-T077N-T078I-N096D-Y103F-G127T- | 0.35 | 0.38 |
| | A128K-S129Q-G165Q-N184Q-A202V-G203E-N217S- | | |
| | S258P-F260W | | |
| CMT24-05546 | P009T-Q017H-V045R-T077N-T078I-N096D-Y103F- | 0.36 | 0.46 |
| | G127T-A128K-S129Q-T161Q-G165Q-S181Q-N184Q- | | |
| | A202V-G203E-N217S-S258P-F260W-K264H | | |
| CMT24-05565 | P009T-Q017H-T077N-T078I-N096D-G127T-A128K- | 0.36 | 0.31 |
| | S129Q-G165Q-N184Q-A202V-G203E-N217S-S258P | | |
| CMT24-05557 | P009T-Q017H-V045R-T077N-T078I-S086D-N096D- | 0.37 | 0.53 |
| | S108Q-T115L-G127T-A128K-S129Q-G165Q-N184Q- | | |
| | A202V-G203E-N217S-S258P | | |
| CMT24-05569 | P009T-Q017H-A068S-T077N-T078I-N096D-S108Q- | 0.37 | 0.49 |
| | T115L-G127T-A128K-S129Q-G165Q-N184Q-A202V- | | |
| | G203E-N217S-S258P-K264H | | |
| CMT24-05555 | P009T-Q017H-V045R-A068S-T077N-T078I-S086D- | 0.38 | 0.54 |
| | N096D-S108Q-T115L-G127T-A128K-S129Q-G165Q- | | |
| | N184Q-A202V-G203E-N217S-S258P | | |
| CMT24-05558 | P009T-Q017H-V045R-A068S-T077N-T078I-S086D- | 0.40 | 0.51 |
| | N096D-S108Q-T115L-G127T-A128K-S129Q-G165Q- | | |
| | N184Q-A202V-G203E-N217S-S258P-F260W | | |
| CMT24-05540 | P009T-Q017H-T077N-T078I-N096D-Y103F-G127T- | 0.44 | 0.58 |
| | A128K-S129Q-T161Q-G165Q-S181Q-N184Q-A202V- | | |
| | G203E-N217S-S258P-F260W | | |
| CMT24-05532 | P009T-Q017H-V045R-T077N-T078I-S086D-N096D- | 0.47 | 0.75 |
| | Y103F-G127T-A128K-S129Q-G165Q-N184Q-A202V- | | |
| | G203E-N217S-S258P | | |
| CMT24-05538 | P009T-Q017H-V045R-T077N-T078I-S086D-N096D- | 0.52 | 0.73 |
| | Y103F-G127T-A128K-S129Q-G165Q-N184Q-A202V- | | |
| | G203E-N217S-S258P-F260W | | |
| CMT24-05556 | P009T-Q017H-V045R-A068S-T077N-T078I-S086D- | 0.52 | 0.66 |
| | N096D-S108Q-T115L-G127T-A128K-S129Q-T161Q- | | |
| | G165Q-S181Q-N184Q-A202V-G203E-N217S-S258P | | |
| CMT24-05587 | P009T-Q017H-T077N-T078I-N096D-Y103F-G127T- | 0.53 | 0.81 |
| | A128K-S129Q-G165Q-N184Q-A202V-G203E-N217S- | | |
| | M221Q-S258P | | |
| CMT24-05559 | P009T-Q017H-V045R-A068S-T077N-T078I-S086D- | 0.56 | 0.61 |
| | N096D-S108Q-T115L-G127T-A128K-S129Q-T161Q- | | |
| | G165Q-S181Q-N184Q-A202V-G203E-N217S-S258P- | | |
| | F260W | | |
| CMT24-05537 | P009T-Q017H-V045R-T077N-T078I-S086D-N096D- | 0.71 | 0.74 |
| | Y103F-G127T-A128K-S129Q-T161Q-G165Q-S181Q- | | |
| | N184Q-A202V-G203E-N217S-S258P | | |
| CMT24-05539 | P009T-Q017H-V045R-T077N-T078I-S086D-N096D- | 0.73 | 0.73 |
| | Y103F-G127T-A128K-S129Q-T161Q-G165Q-S181Q- | | |
| | N184Q-A202V-G203E-N217S-S258P-F260W | | |

TABLE 8
Stability evaluation of AprL variants reported as fraction of residual activity
when tested under conditions (temperature and detergent) described.

| | | 10% | 20% |
|---|---|---|---|
| | | PNB | CNS |
| Enzyme Name | Substitutions vs AprL | 72° C. | 70° C. |
| AprL-WT | none | 0.00 | 0.00 |
| CMT24 | A068S-T077N-T078I-G127S-A128P-G165Q-N184Q- | 0.01 | 0.01 |
| | A202V-N217S-S258P | | |
| CMT24-05681 | P009T-Q017H-T077N-T078I-N096D-Y103F-G127T- | 0.46 | 0.51 |
| | A128K-S129Q-G165Q-N184Q-A202V-G203E-N217S | | |
| CMT24-05693 | P009T-Q017H-T077N-T078I-N096D-Y103F-G127T- | 0.39 | 0.43 |
| | A128K-S129Q-G165Q-N184Q-A202V-G203E-S258P | | |
| CMT24-05687 | P009T-Q017H-V045R-T077N-T078I-S086D-N096D-Y103F- | 0.59 | 0.73 |
| | G127T-A128K-S129Q-G165Q-N184Q-A202V-G203E- | | |
| | N217S | | |
| CMT24-05669 | P009T-Q017H-V045R-T077N-T078I-S086D-N096D-Y103F- | 0.58 | 0.47 |
| | G127T-A128K-S129Q-G165Q-N184Q-A202V-G203E- | | |
| | S258P | | |
| CMT24-05694 | P009T-Q017H-V045R-T077N-T078I-N096D-Y103F- | 0.49 | 0.48 |
| | G127T-A128K-S129Q-S155E-G165Q-N184Q-A202V- | | |
| | G203E-N217S | | |
| CMT24-05677 | P009T-Q017H-V045R-T077N-T078I-N096D-Y103F- | 0.43 | 0.40 |
| | G127T-A128K-S129Q-S155E-G165Q-N184Q-A202V- | | |
| | G203E-S258P | | |
| CMT24-05670 | P009T-Q017H-V045R-T077N-T078I-S086D-N096D-Y103F- | 0.72 | 0.83 |
| | G127T-A128K-S129Q-T161Q-G165Q-S181Q-N184Q- | | |
| | A202V-G203E-N217S | | |
| CMT24-05699 | P009T-Q017H-V045R-T077N-T078I-S086D-N096D-Y103F- | 0.66 | 0.74 |
| | G127T-A128K-S129Q-T161Q-G165Q-S181Q-N184Q- | | |
| | A202V-G203E-S258P | | |
| CMT24-05685 | P009T-Q017H-V045R-A068S-T077N-T078I-N096D-S108Q- | 0.54 | 0.51 |
| | T115L-G127T-A128K-S129Q-G165Q-N184Q-A202V- | | |
| | G203E-N217S-K264H | | |
| CMT24-05689 | P009T-Q017H-V045R-T077N-T078I-S086D-N096D-Y103F- | 0.76 | 0.83 |
| | G127T-A128K-S129Q-T161Q-G165Q-S181Q-N184Q- | | |
| | A202V-G203E-N217S-F260W | | |
| CMT24-05673 | P009T-Q017H-V045R-T077N-T078I-S086D-N096D-Y103F- | 0.60 | 0.62 |
| | G127T-A128K-S129Q-T161Q-G165Q-S181Q-N184Q- | | |
| | A202V-G203E-S258P-F260W | | |

This study of AprL subtilisin variants has identified numerous amino acid substitutions that impart significant improvement in stability in detergent at elevated temperatures, as can be seen on data in Tables 3, 4, 5, 6, 7, and 8. The parent AprL-WT (wildtype) and a previously studied AprL variant, CMT24 (SEQ ID NO:8) were used for comparison. The detergents and enzyme compositions evaluated lack protease stabilizers.

### Example 4

### Time-Course Stability Evaluation of AprL Variants in Liquid Detergent

A time course study of enzyme stability in HDL detergent was performed using the Test Detergent A to compare the stability of PREFERENZ® P 100, the previously disclosed subtilisin variant CMT24 (SEQ ID NO:8) (variant disclosed as SQCBV419 in patent application WO2017/210295) and AprL variants described herein. Proteases were dosed at 0.07 wt % CMT24-05693, 0.07 wt % CMT24 and 0.04 wt % PREFERENZ® P 100 based on active protein in detergent, and mixed at ambient room temperature. The samples were subsequently stored in an incubator with rotation, at 37° C. Aliquots were collected at the start of the incubation (time zero) and at additional times up to 8 weeks. The remaining enzyme activity was measured using the AAPF substrate as described in Example 2, and results are reported as percent (%) residual activity. Table 9 shows the results of a comparison at 1, 4 and 8 weeks of the variant CMT24-05693 and reference molecules CMT24 and PREFERENZ® P 100. The new variant CMT24-05693 displays significant improvement in stability as compared to both reference enzymes.

TABLE 9
Stability evaluation in Test Detergent A, tested under
conditions (temperature and detergent) described.
Results are shown as percent residual activity.

| Incubation | | | |
|---|---|---|---|
| time [wks] | CMT24-05693 | CMT24 | PREFERENZ ® P 100 |
| 0 | 100 | 100 | 100 |
| 1 | 89 | 56 | 15 |
| 4 | 89 | 26 | 0 |
| 8 | 68 | 17 | — |

Additional AprL variants were evaluated versus the reference enzyme CMT24 to determined percent residual activity after incubation over time, using the method described above. Results are shown on Tables 10 and 11 for samples tested in Persil Non Bio (PNB) detergent. Culture supernatants from cells expression of the various variants were used for this study. Variants shown on Table 10 were expressed in a B. subtilis host, and variants on Table 11 were expressed in a B. licheniformis host. For data on Table 10, protease dosages in the detergent were in the range of 0.02% to 0.08%. Variant CMT24-07674 consists of substitution P009T-Q017H-T077N-T078I-N096D-G127T-A128K-S129Q-G165Q-N184Q-A202V-G203E-S258P. Variant CMT24-07675 consists of substitutions P009T-Q017H-T077N-T078I-N096D-G127T-A128K-S129Q-G165Q-N184Q-A202V-G203E-N217S. Variant CMT24-02667 consists of substitutions P009T-Q017H-V045R-T077N-T078I-N096D-S108Q-G127T-A128P-G165Q-N184Q-A202V-G203E-N217S-S258P, Variant CMT24-02843 consists of substitutions Q017H-T077N-T078I-N096D-S108Q-G117R-G127T-A128P-G165Q-N184Q-A202V-G203E-N217S-S258P, Variant CMT24-02609 consists of substitutions P009T-Q017H-V045R-T077N-T078I-N096D-G127T-A128P-G165Q-N184Q-A202V-G203E-N217S-S258P, and variant CMT-05698 consists of substitutions P009T-Q017H-V045R-A068S-T077N-T078I-N096D-S108Q-T115L-G127T-A128K-S129Q-G165Q-N184Q-A202V-G203E-S258P-K264H, In all cases the substitutions are with respect to AprL WT (SEQ ID NO:1). As shown on Tables 10 and 11, all new AprL variants display significantly higher stability in HDL detergent when compared to reference enzyme CMT24.

TABLE 10
Stability in Persil Non Bio detergent of AprL variants
produced in B. subtilis. AprL variants were evaluated
alongside reference protein CMT24 and residual activity
are reported as percent of non-stressed activity.

Days of incubation at 37° C.

| Sample ID | 7 | 14 | 27 | 56 |
|---|---|---|---|---|
| CMT-24 | 82 | 79 | 65 | 53 |
| CMT24-02633 | 89 | 94 | 87 | 81 |
| CMT24-02667 | 89 | 93 | 88 | 82 |
| CMT24-02843 | 83 | 89 | 85 | 87 |
| CMT24-03184 | 89 | 95 | 89 | 94 |
| CMT24-03196 | 86 | 94 | 88 | 86 |
| CMT24-05547 | 87 | 96 | 90 | 93 |
| CMT24-05555 | 87 | 97 | 93 | 96 |
| CMT24-02609 | 92 | 92 | 81 | 73 |
| CMT24-02825 | 86 | 86 | 74 | 65 |

Table 11 below shows several instances when the incubation period (shown below in number of days) was similar but not identical for some tests, and the asterisk (*) is used to indicate the number of days of the tests in the respective column.

TABLE 11
Stability in Persil Non Bio detergent of AprL variants produced
in B. licheniformis. AprL variants were evaluated alongside
reference protein CMT24 and residual activity results are
reported as percent of non-stressed activity.

Days of incubation at 37° C.

| Sample ID | 7 | 13 | 19*/21 | 40/41* | 54 |
|---|---|---|---|---|---|
| CMT24 | 66 | 55 | 53 | 33* | 28 |
| CMT24-05693 | 93 | 90 | | | 70 |
| CMT24-05681 | 94 | 90 | | | 63 |
| CMT24-05687 | 94 | 92 | | | 68 |
| CMT24-05532 | 91 | | 82* | 77 | |
| CMT24-05539 | 93 | | 86* | 80 | |
| CMT24-05565 | 92 | | 82* | 76 | |
| CMT24-05582 | 91 | | 84* | 80 | |
| CMT24-03330 | 95 | | 88 | 75* | |
| CMT24-05698 | 91 | | 89 | 76* | |
| CMT24-05685 | 92 | | 84 | 70* | |
| CMT24-07674 | 94 | | 92 | 78* | |
| CMT24-07675 | 92 | | 81 | 65* | |

### Example 5

### Aged Laundry Cleaning Performance of Various AprL Variants

The cleaning performance of the subtilisin variants were tested using a process to age the detergent prior to measuring the cleaning activity. The protocol for measuring aged laundry cleaning is as follows.

Samples were prepared by adding the AprL subtilisin variants at an equal active protein basis equivalent to 1.9 ppm in the wash liquor, which also included 0.11 ppm of amylase PREFERENZ S210, and 0.11 ppm of PspMan138 variant mannanase TL1219 (U.S. provisional application 63/403,332, filed Sep. 2, 2022) into 100% Persil Non Bio detergent. The detergent, dosed at 2.7 g per liter of wash liquor, with added enzymes was mixed end-over-end for at least 1 hour. Samples were then stored in an incubator at 37° C. and aged for 4 weeks. After aging, the samples were evaluated for cleaning performance in Miele Professional wash machines (Model PW6065 Plus). The wash process takes place over 55 minutes, with a main wash of 25 minutes at 30° C. To the wash, 250 ppm of 3:1 Ca:Mg water hardness and about 3 kilograms total of clean ballast, technical stain monitors, and synthetic soil ballast were added. Following the wash process, technical stains were dried using the gentle cycle. Cleaning was evaluated on dried stains photometrically using the MACH-5. L*a*b* measurements of each stain taken before and after the wash process were used to determine the percent Stain Removal Index (% SRI) for each aged sample. The % SRI was calculated using the following formula, where w is the white standard, i is the stain prior to the wash, and f is the stain after the wash.

$$\% SRI dE = \frac{( White - Before ) - ( White - After )}{( White - Before )} = \frac{\begin{matrix}\sqrt{{( {L}_{w} - {L}_{i} )}^{2} + {( {a}_{w} - {a}_{i} )}^{2} + {( {b}_{w} - {b}_{i} )}^{2}} - \\ \sqrt{{( {L}_{w} - {L}_{f} )}^{2} + {( {a}_{w} - {a}_{f} )}^{2} + {( {b}_{w} - {b}_{f} )}^{2}}\end{matrix}}{\sqrt{{( {L}_{w} - {L}_{i} )}^{2} + {( {a}_{w} - {a}_{i} )}^{2} + {( {b}_{w} - {b}_{i} )}^{2}}}$$

The stains used in this evaluation are as follows: C-03 Chocolate milk/soot, C-05 Blood/milk/ink, C-S-07 Grass, and C-S-39 Full egg/with pigment/aged by heat. Technical stains were purchased from Center for Testmaterials BV, Vlaardingen, Netherlands.

Table 12 shows the % SRI (percent Stain Removal Index) of aged samples of various AprL variants compared to the reference molecule CMT24 across the test stain set.

TABLE 12
Aged performance reported of various AprL variants compared
to the reference molecule CMT24 across test stain set.

% SRI aged cleaning for each stain

| Enzyme | C-03 | C-05 | C-S-07 | C-S-39 |
|---|---|---|---|---|
| CMT24 | 15% | 4% | 25% | 24% |
| CMT24-03330 | 17% | 6% | 29% | 30% |
| CMT24-05693 | 17% | 7% | 27% | 30% |
| CMT24-05681 | 17% | 6% | 27% | 30% |
| CMT24-05687 | 17% | 6% | 29% | 30% |

As show in Table 12, all AprL variants provided improved aged cleaning when compared to reference protein CMT24 (SEQ ID NO:8) across multiple protease-sensitive stains.

### Example 6

### Full Scale Cleaning Performance of AprL Variants

Cleaning performance was measured on enzyme responsive stains that are commercially available from Center for Testmaterials (CFT BV, The Netherlands). Testing was performed with Miele W1935 WPS Ecoline laundry washing machines using the “Cotton, Short” wash cycle at 20° C. and 30° C.

The wash process takes place over 109 minutes, with a main wash of 50 minutes. The washing load composed of 3 kg cotton ballast, synthetic soil ballast (4×SBL2004) soil ballast and technical stains. The water was maintained at 14° GH (using 3Ca: 1 Mg). Stain Removal Index (SRI) % was calculated as given in Example 5. The technical stains used in this study are as follows: CS-01 (Blood aged on cotton), CS-100 (Chocolate rice pudding, aged on cotton), C-S-39 (Full egg with carbon black, aged on cotton), C-S-45 (Chocolate soymilk drink, aged on cotton) and CS-07 (Grass on cotton). Technical stains were purchased from Center for Testmaterials BV, Vlaardingen, Netherlands.

For the study shown on Table 13, the samples were prepared by adding protease at an equal active protein basis equivalent to 0.95 ppm in the wash liquor, which also included 0.07 ppm of amylase PREFERENZ S210, and 0.01 ppm of mannanase PREFERENZ M 100 into 100% Persil Non Bio (PNB) detergent. The detergent, dosed at 2.8 g per liter of wash liquor, with added enzymes was mixed for at least 1 hour.

Table 13 shows the results of testing AprL variant CMT24-05693 and CMT24 reference protease as described above. The standard deviation (StdDev) for the results, which are an average of 4 repeat (2 stains x 2 machines) are shown at the bottom of the table. Variant CMT24-05693 shows significant benefit across stains compared to commercially relevant reference enzyme CMT24. For direct comparison of the new variant CMT24-05693 to the reference molecule CMT24, performance index (PI) values were calculated by subtracting the nil protease % SRI responses from the enzyme added results, and then calculating the ratio of delta % SRI for CMT24-05693 versus the CMT24 reference. Results are shown in column labeled PI. A PI of 1.0 indicates equivalent performance, while PIs of 1.1 and greater represent a significant cleaning benefit.

TABLE 13
Cleaning performance of proteases in Persil Non Bio evaluated
at 20° C. across multiple stains. Results are reported
as % SRI (stain removal index), and PI (performance index)
of variant CMT24-05693 to reference CMT24.

| | Average of % SRI (dE) | | | |
|---|---|---|---|---|
| Stains | nil protease | CMT24 | CMT24-05693 | PI |
| CS-01 | 35 | 39 | 43 | 2.0 |
| CS-100 | 27 | 50 | 56 | 1.3 |
| C-S-39 | 18 | 32 | 33 | 1.1 |
| C-S-45 | 7.5 | 16 | 20 | 1.5 |
| CS-07 | 27 | 32 | 36 | 1.8 |

| | StdDev of % SRI (dE) | | |
|---|---|---|---|
| | nil protease | CMT24 | CMT24-05693 |
| CS-01 | 2.3 | 1.6 | 0.9 |
| CS-100 | 0.7 | 1.7 | 1.0 |
| C-S-39 | 0.8 | 2.0 | 0.8 |
| C-S-45 | 0.7 | 1.8 | 0.7 |
| CS-07 | 2.6 | 1.1 | 1.2 |

An additional cleaning performance was performed at 30° C. under the same washing conditions described above and reported on Table 14. The technical stains used in this study are as follows: C-03 (Chocolate milk with carbon black on cotton), C-05 (Blood/milk/ink on cotton), C-11 (Milk with carbon black on cotton), CS-01 (Blood, aged on cotton), CS-07 (Grass on cotton), C-S-100 (Chocolate rice pudding, aged on cotton), C-S-39 (Full egg with carbon black, aged on cotton), EMPA 116 (Blood/milk/ink on polycotton), and KCS-01 (Blood, aged on polyester/cotton). The samples were prepared by adding protease at an equal active protein basis equivalent to 2.85 ppm in the wash liquor, which also included 0.07 ppm of amylase PREFERENZ S210, and 0.01 ppm of mannanase PREFERENZ M 100 into 100% Persil Non Bio detergent. The detergent, dosed at 2.8 g per liter of wash liquor, with added enzymes was mixed for at least 1 hour.

Table 14 shows the results of testing several AprL variants and the CMT24 protease at 30° C. on various technical stains, in the presence of amylase and mannanase. The standard deviation for each data set is shown at the bottom of the table. The cumulative benefit across stains was calculated and shown as Total, and the PI values are shown for each evaluation. The results show improved benefits for all the AprL variants compared to the CMT24 protease.

TABLE 14
Cleaning performance of proteases in Persil Non Bio evaluated at 30°
C. across multiple stains. Results reported as % SRI (stain removal
index) and PI (performance index) relative to reference CMT24.

| | Average of % SRI (dE) | | | | | PI (Performance Index) | | |
|---|---|---|---|---|---|---|---|---|
| | nil | | CMT24- | CMT24- | CMT24- | CMT24- | CMT24- | CMT24- |
| Stain | protease | CMT24 | 03330 | 05693 | 07674 | 03330 | 05693 | 07674 |
| C-03 | 11 | 23 | 25 | 27 | 26 | 1.2 | 1.3 | 1.3 |
| C-05 | 10 | 26 | 30 | 30 | 29 | 1.3 | 1.3 | 1.2 |
| C-11 | 4 | 29 | 34 | 36 | 33 | 1.2 | 1.3 | 1.2 |
| CS-01 | 23 | 35 | 40 | 40 | 40 | 1.4 | 1.4 | 1.4 |
| CS-07 | 37 | 40 | 42 | 43 | 41 | 1.7 | 2.0 | 1.3 |
| C-S-100 | 31 | 58 | 61 | 61 | 61 | 1.1 | 1.1 | 1.1 |
| C-S-39 | 14 | 37 | 41 | 40 | 39 | 1.2 | 1.1 | 1.1 |
| EMPA | 23 | 44 | 48 | 46 | 46 | 1.2 | 1.1 | 1.1 |
| 116 | | | | | | | | |
| KCS-01 | 26 | 45 | 50 | 51 | 49 | 1.3 | 1.3 | 1.2 |
| Total* | 179 | 338 | 370 | 373 | 363 | | | |

| | StdDev of % SRI (dE) | | | | |
|---|---|---|---|---|---|
| | nil | | CMT24- | CMT24- | CMT24- |
| | protease | CMT24 | 03330 | 05693 | 07674 |
| C-03 | 0.6 | 1.3 | 2.1 | 0.9 | 1.3 |
| C-05 | 0.3 | 1.5 | 1.2 | 1.2 | 1.6 |
| C-11 | 1 | 3 | 1.8 | 2.5 | 2.4 |
| CS-01 | 0.6 | 1.2 | 1.8 | 1.8 | 2 |
| CS-07 | 2.2 | 0.9 | 2.4 | 2.3 | 1.3 |
| C-S-100 | 0.4 | 1.1 | 0.9 | 0.8 | 0.6 |
| C-S-39 | 0.9 | 1 | 1.3 | 0.8 | 0.8 |
| EMPA | 1 | 0.7 | 0.7 | 1.9 | 1.4 |
| 116 | | | | | |
| KCS-01 | 0.9 | 1.3 | 3.3 | 2.4 | 0.8 |
| Total* | 8 | 12 | 15 | 15 | 12 |

### Example 7

### Cleaning Performance and Stability in Detergent of Additional AprL Variants

Additional AprL variants shown in Table 15 were generated from expression in B. subtilis host as described in Example 1 and clarified culture supernatants were used to carry out tests in microtiter scale. Detergent stability was tested in 10% Test detergent A (at 45° C.) and 20% CNS (at 48° C.) using the method described in Example 2. The cleaning performance was tested as described below. The protease variants were tested for cleaning performance relative to a reference AprL-WT parent (SEQ ID NO:1).

### Cleaning Performance Assay

For cleaning performance assays, the Test detergent A was diluted to 6.0 g/L in 5 mM HEPES (pH 8.2) with a water hardness of 6 gpg. The technical EMPA-116 (blood/milk/ink on woven cotton), was purchased from Center for Testmaterials BV, Vlaardingen, Netherlands. The swatches were punched into small circular swatches and distributed into microtiter plates (MTPs). The microswatch-containing MTPs were first filled with detergent solution. Afterwards, quantities of the parent enzyme and variants were added to a final volume of 200 microliters. Assays were carried out at 25° C. for 25 minutes with gentle shaking. Following the incubation period, 50 microliters of supernatant was transferred to a fresh MTP and absorbance was read at 600 nm using a SpectraMax plate reader. Absorbance results were obtained by subtracting the value for a blank control (no enzyme) from each sample value. For each condition and subtilisin variant, a cleaning performance index (PI) was calculated by dividing the blank subtracted absorbance of the variant by that of the parent protease at the same concentration. The blank subtracted absorbance value for the parent protease at the corresponding concentration of the variant was determined using a standard curve of the parent protease, which was included in the test and was generated using a Langmuir fit or Hill Sigmoidal fit, as appropriate.

The proteolytic activity of the AprL subtilisin variants was also tested by measuring the hydrolysis of di-methyl casein (DMC) substrate. For the DMC assay, the reagent solutions used were: 2.5% di-methyl casein (DMC, Sigma C-9801) dissolved in carbonate buffer (100 mM Sodium Carbonate pH 9.2+100 mM Sodium Chloride) and 0.075% TNBSA (2,4,6-trinitrobenzene sulfonic acid, Thermo Scientific) diluted in carbonate buffer. MTPs (Greiner PS-microwell 384) were filled with 27 uL DMC substrate followed by the addition of 27 ul of TNBSA. Reactions were started by addition of 6 μL of the appropriately diluted protease culture supernatant and mixing. After 3 min incubation at RT, the proteolytic activity was measured at 405 nm over 3 minuntes using a SpectraMax plate reader in kinetic mode at RT. The relative proteolytic activity of the proteases was captured as mOD*min-1.

Th net charge for each variant was calculated using the pKₐ values of amino acids, as reported in Hass and Mulder (Hass, M. A. S and Mulder, F. A. A (2015) Annu. Rev. Biophys. 44:53-75) at pH S with respect to parent AprL set at zero.

Table 15 provides the results for proteolytic activity, cleaning performance, detergent stability, and calculated net charge at pH 8 of AprL variants compared to reference. AprL parent.

TABLE 15
Results of evaluation of AprL variants relative to AprL-WT. Proteolytic activity on DMC,
detergent stability reported as fraction of residual activity (temperature and detergents
as described), cleaning performance on EMPA116 stain in Test Detergent A reported as
PI (performance index) were determined, and net charges at pH 8 were calculated.

| | | Stability | | | Cleaning | |
|---|---|---|---|---|---|---|
| | | (fraction residual activity) | | Proteolytic | (PI) | |
| | | Test | | Activity | Test | Net |
| Enzyme | | Detergent A | CNS | DMC | detergent A | charge |
| Name | Substitutions vs AprL | at 45° C. | at 48° C. | substrate | EMPA116 | at pH 8 |
| AprL-WT | none | 0.4 | 0.0 | 42 | 1 | 0 |
| CMT24- | Q017H-N096D-G127T | 0.9 | 0.2 | 75 | 1.7 | −1 |
| 07804 | | | | | | |
| CMT24- | Q017H-N096D-Y103F- | 1.0 | 0.7 | 49 | 1.1 | −1 |
| 07740 | A202V | | | | | |
| CMT24- | V045R-S155E-A202V- | 1.0 | 0.7 | 47 | 1.1 | −1 |
| 07748 | G203E | | | | | |
| CMT24- | G127T-A128K-S129Q- | 0.9 | 0.2 | 79 | 1.1 | 1 |
| 07948 | N184Q | | | | | |
| CMT24- | P009T-Y103F-A202V- | 0.9 | 0.9 | 41 | 1.1 | −1 |
| 07956 | G203E | | | | | |
| CMT24- | N096D-S100E-G127T- | 1.0 | 0.7 | 36 | 1.3 | −2 |
| 07964 | A202V | | | | | |
| CMT24- | V087A-S155E-G165Q | 0.9 | 0.8 | 49 | 1.3 | −1 |
| 07757 | | | | | | |
| CMT24- | P009T-T078I-Y103F-G127T | 0.9 | 0.5 | 65 | 1.3 | 0 |
| 07709 | | | | | | |
| CMT24- | V087A-A202V-G203E | 0.9 | 0.8 | 41 | 1.2 | −1 |
| 07789 | | | | | | |
| CMT24- | V045R-T161Q-S181Q- | 0.9 | 0.4 | 56 | 1 | 0 |
| 07877 | G203E | | | | | |
| CMT24- | S155E-G203E | 0.8 | 0.5 | 48 | 1.3 | −2 |
| 07981 | | | | | | |
| CMT24- | S155E-M221Q | 0.7 | 0.2 | 62 | 1.2 | −1 |
| 07989 | | | | | | |
| CMT24- | T078I-Y103F | 0.6 | 0.2 | 53 | 1.2 | 0 |
| 07758 | | | | | | |
| CMT24- | N096D-S100E-G127T- | 0.9 | 0.2 | 38 | 1.4 | −2 |
| 07702 | N217S | | | | | |
| CMT24- | G127T-A128K-S129Q | 0.6 | 0.1 | 74 | 1.3 | 1 |
| 07782 | | | | | | |
| CMT24- | T115L-G127T | 0.6 | 0.0 | 65 | 1.3 | 0 |
| 07726 | | | | | | |
| CMT24- | P009T-T078I-N096D-A202V | 1.1 | 0.5 | 39 | 1 | −1 |
| 07742 | | | | | | |
| CMT24- | V045R-S086E-S155E- | 0.8 | 0.6 | 36 | 1.2 | −1 |
| 07974 | A202V | | | | | |
| CMT24- | V045R-T078I-Y103F-A202V | 0.8 | 0.5 | 44 | 1.1 | 1 |
| 07775 | | | | | | |
| CMT24- | P009T-A202V-G203E- | 1.0 | 0.9 | 44 | 1.1 | −1 |
| 07703 | M221Q | | | | | |
| CMT24- | A202V-G203E-M221Q- | 1.3 | 1.0 | 46 | 0.9 | −2 |
| 07727 | K264H | | | | | |
| CMT24- | N096D-G127T | 0.6 | 0.1 | 74 | 1.5 | −1 |
| 07807 | | | | | | |
| CMT24- | P009T-Q017H-Y103F- | 0.8 | 0.4 | 39 | 0.9 | 0 |
| 07743 | A202V | | | | | |
| CMT24- | V045R-G127T-A128P- | 0.6 | 0.0 | 62 | 1.5 | 1 |
| 07935 | S129Q | | | | | |
| CMT24- | S100E-G127T-A202V | 0.8 | 0.4 | 37 | 1.1 | −1 |
| 07863 | | | | | | |
| CMT24- | A202V-M221Q | 1.0 | 0.5 | 41 | 1 | 0 |
| 07951 | | | | | | |
| CMT24- | S100N-Y103I-G127T | 0.5 | 0.1 | 54 | 1.4 | 0 |
| 07879 | | | | | | |
| CMT24- | G127T-A128K-S129Q- | 0.8 | 0.4 | 52 | 1.3 | 1 |
| 07784 | N217S | | | | | |
| CMT24- | G127S-A128K-S129Q- | 0.8 | 0.4 | 48 | 0.7 | 1 |
| 07752 | A202V | | | | | |
| CMT24- | T078I-N096D | 0.6 | 0.1 | 49 | 1.1 | −1 |
| 07856 | | | | | | |
| CMT24- | A128K-S155E | 0.8 | 0.1 | 58 | 1 | 0 |
| 07936 | | | | | | |
| CMT24- | N096D-Y103F-G165Q | 0.9 | 0.3 | 64 | 1.1 | −1 |
| 07880 | | | | | | |
| CMT24- | S086E-T077N-T078I | 0.7 | 0.2 | 45 | 1.1 | −1 |
| 07976 | | | | | | |
| CMT24- | V045R-T078I-S086E-A202V | 0.9 | 0.5 | 42 | 1.1 | 0 |
| 07785 | | | | | | |
| CMT24- | S155E-A202V | 0.9 | 0.4 | 33 | 1.1 | −1 |
| 07737 | | | | | | |
| CMT24- | N096D-S100E-G127S | 0.7 | 0.0 | 49 | 1.2 | −2 |
| 07745 | | | | | | |
| CMT24- | T078I-G127T-A128K-S129Q | 0.9 | 0.5 | 48 | 1.1 | 1 |
| 07921 | | | | | | |
| CMT24- | G127S-A128K-S129Q | 0.8 | 0.2 | 63 | 0.7 | 1 |
| 07873 | | | | | | |
| CMT24- | Q017H-S086E-A202V- | 0.9 | 0.9 | 38 | 1 | −2 |
| 07953 | G203E | | | | | |
| CMT24- | T161Q-S181Q-A202V- | 1.0 | 0.9 | 46 | 1 | −1 |
| 07889 | G203E | | | | | |
| CMT24- | V045R-N096D-S100E | 0.5 | 0.0 | 48 | 1.4 | −1 |
| 07786 | | | | | | |
| CMT24- | V045R-Y103F-G127T- | 0.9 | 0.4 | 48 | 1.4 | 1 |
| 07794 | A202V | | | | | |
| CMT24- | N096D-S100E-G117R- | 0.5 | 0.0 | 47 | 1.4 | −1 |
| 07722 | G127T | | | | | |
| CMT24- | V087A-G165Q-A202V | 1.0 | 0.8 | 48 | 0.9 | 0 |
| 07746 | | | | | | |
| CMT24- | G127T-A128P-S129Q | 0.5 | 0.1 | 61 | 1.5 | 0 |
| 07826 | | | | | | |
| CMT24- | V087A-M221Q | 0.9 | 0.3 | 59 | 1.1 | 0 |
| 07922 | | | | | | |
| CMT24- | T115L-A202V-G203E | 0.9 | 0.7 | 46 | 1 | −1 |
| 07962 | | | | | | |
| CMT24- | V045R-T078I-N096D- | 0.9 | 0.6 | 43 | 1.1 | 0 |
| 07970 | A202V | | | | | |
| CMT24- | G127T-A128R-S129Q- | 1.0 | 0.6 | 76 | 1 | 1 |
| 07771 | G165Q | | | | | |
| CMT24- | S100E-Y103I | 0.4 | 0.0 | 49 | 1.5 | −1 |
| 07779 | | | | | | |
| CMT24- | S086E-S155E-A202V- | 1.0 | 0.9 | 38 | 1.2 | −3 |
| 07787 | G203E | | | | | |
| CMT24- | N096D-Y103I-G127T | 0.6 | 0.0 | 76 | 1.5 | −1 |
| 07715 | | | | | | |
| CMT24- | Q017H-N096D-Y103F | 0.5 | 0.0 | 55 | 1.1 | −1 |
| 07843 | | | | | | |
| CMT24- | V045R-T078I-N096D-Y103F | 0.7 | 0.2 | 51 | 1.2 | 0 |
| 08076 | | | | | | |
| CMT24- | S108Q-G127T | 0.8 | 0.2 | 67 | 1.3 | 0 |
| 08084 | | | | | | |
| CMT24- | T078I-S086E-A202V-G203E | 1.0 | 1.1 | 46 | 0.8 | −2 |
| 08020 | | | | | | |
| CMT24- | T078I-Y103F-G127T-A202V | 0.9 | 0.6 | 22 | 1.6 | 0 |
| 08100 | | | | | | |
| CMT24- | S181E-A202V-G203E- | 1.0 | 1.0 | 46 | 0.8 | −3 |
| 08028 | S258D | | | | | |
| CMT24- | N096D-A202V-G203E | 0.9 | 0.8 | 43 | 1.2 | −2 |
| 08116 | | | | | | |
| CMT24- | N096D-Y103I-G127T- | 0.8 | 0.2 | 81 | 1.2 | −1 |
| 08052 | M221Q | | | | | |
| CMT24- | V087A-G165Q-A202V- | 1.2 | 1.0 | 58 | 1 | −1 |
| 08060 | G203E | | | | | |
| CMT24- | P009T-G127T-A128K- | 0.8 | 0.2 | 76 | 1.2 | 1 |
| 08148 | S129Q | | | | | |
| CMT24- | S100E-G127T | 0.8 | 0.0 | 53 | 1.3 | −1 |
| 08005 | | | | | | |
| CMT24- | S100N-M221Q | 0.4 | 0.1 | 71 | 1.2 | 0 |
| 08093 | | | | | | |
| CMT24- | G127T-A128K-S129Q- | 0.9 | 0.5 | 50 | 0.9 | 1 |
| 08021 | A202V | | | | | |
| CMT24- | V087A-S100N | 0.5 | 0.0 | 62 | 1.1 | 0 |
| 08101 | | | | | | |
| CMT24- | S100E-G117R-G127T | 0.4 | 0.0 | 56 | 1.3 | 0 |
| 08141 | | | | | | |
| CMT24- | T077N-G127T-A128K- | 0.8 | 0.3 | 67 | 0.7 | 1 |
| 07998 | S129Q | | | | | |
| CMT24- | Q017H-N096D-G127T- | 0.9 | 0.4 | 43 | 1.6 | −1 |
| 08014 | A202V | | | | | |
| CMT24- | N096D-A202V-M221Q | 0.8 | 0.6 | 43 | 1.2 | −1 |
| 08094 | | | | | | |
| CMT24- | V045R-N096D-Y103F- | 0.7 | 0.0 | 72 | 1.3 | 0 |
| 08022 | G127T | | | | | |
| CMT24- | N096D-G127T-G203E | 0.9 | 0.4 | 77 | 1.2 | −2 |
| 08030 | | | | | | |
| CMT24- | V045R-S086E-A202V- | 0.9 | 0.9 | 43 | 1 | −1 |
| 08070 | G203E | | | | | |
| CMT24- | T078I-S086E-S155E-G203E | 1.0 | 0.7 | 52 | 0.9 | −3 |
| 08150 | | | | | | |
| CMT24- | V045R-M221Q | 0.6 | 0.1 | 34 | 0.8 | 1 |
| 07999 | | | | | | |
| CMT24- | Q017H-N096D-Y103F- | 0.7 | 0.1 | 38 | 1.4 | −1 |
| 08079 | G127T | | | | | |
| CMT24- | Q017H-Y103F-G127T- | 0.8 | 0.7 | 50 | 1.3 | 0 |
| 08015 | A202V | | | | | |
| CMT24- | T078I-A202V-M221Q | 1.0 | 0.6 | 39 | 1.1 | 0 |
| 08023 | | | | | | |
| CMT24- | V045R-N096D-G127T | 0.6 | 0.0 | 62 | 1.4 | 0 |
| 08103 | | | | | | |
| CMT24- | V045R-N096D-G127T- | 0.9 | 0.3 | 48 | 1.6 | 0 |
| 08119 | A202V | | | | | |
| CMT24- | P009T-T078I-Y103F-A202V | 1.0 | 0.5 | 36 | 0.9 | 0 |
| 08071 | | | | | | |
| CMT24- | S100N-T115L | 0.4 | 0.0 | 63 | 0.9 | 0 |
| 08112 | | | | | | |
| CMT24- | S086E-A202V | 1.2 | 0.5 | 35 | 0.9 | −1 |
| 08128 | | | | | | |
| CMT24- | N096D-Y103F-G127T | 0.7 | 0.0 | 67 | 1.3 | −1 |
| 08064 | | | | | | |
| CMT24- | T078I-N096D-G127T | 0.9 | 0.4 | 64 | 1.6 | −1 |
| 08144 | | | | | | |
| CMT24- | T115L-G165Q-A202V | 1.2 | 0.6 | 50 | 1.1 | 0 |
| 08152 | | | | | | |
| CMT24- | T078I-S086E | 0.5 | 0.1 | 42 | 1 | −1 |
| 08105 | | | | | | |
| CMT24- | V045R-T078I-N096D- | 0.7 | 0.4 | 67 | 1.3 | 0 |
| 08033 | G127T | | | | | |
| CMT24- | P009T-N096D-G127T | 0.7 | 0.0 | 64 | 1.5 | −1 |
| 08137 | | | | | | |
| CMT24- | G127T-A128R-S129Q- | 1.0 | 0.5 | 49 | 0.9 | 1 |
| 08153 | A202V | | | | | |
| CMT24- | T078I-N096D-A202V | 1.1 | 0.5 | 34 | 1.4 | −1 |
| 08130 | | | | | | |
| CMT24- | N096D-Y103I-G203E | 1.1 | 0.4 | 50 | 1.3 | −2 |
| 08091 | | | | | | |
| CMT24- | G127S-A128P-S129Q- | 0.9 | 0.6 | 51 | 1.2 | 0 |
| 08099 | A202V | | | | | |
| CMT24- | N096D-G127T-N217S | 0.9 | 0.3 | 49 | 1.5 | −1 |
| 08107 | | | | | | |
| CMT24- | T077N-T078I-M221Q | 0.6 | 0.2 | 52 | 1.1 | 0 |
| 08131 | | | | | | |
| CMT24- | M221Q | 0.5 | 0.1 | 49 | 0.9 | 0 |
| 08158 | | | | | | |
| CMT24- | V045R-T078I | 0.5 | 0.1 | 52 | 1 | 1 |
| 07812 | | | | | | |
| CMT24- | A024Q-T078I-A202V | 0.8 | 0.5 | 39 | 1 | 0 |
| 07924 | | | | | | |
| CMT24- | A202V-G203N | 0.8 | 0.4 | 38 | 0.9 | 0 |
| 07852 | | | | | | |
| CMT24- | P009T-T077N-T078I | 0.5 | 0.1 | 44 | 1.1 | 0 |
| 07876 | | | | | | |
| CMT24- | G203E-K264H | 1.0 | 0.6 | 51 | 1.1 | −2 |
| 07892 | | | | | | |
| CMT24- | T077N-T078I-G165Q- | 1.0 | 0.8 | 45 | 1 | 0 |
| 07941 | A202V | | | | | |
| CMT24- | V045R-T077N-G165Q- | 0.9 | 0.7 | 50 | 0.9 | 1 |
| 07957 | A202V | | | | | |
| CMT24- | G165Q-A202V-G203E- | 1.0 | 0.9 | 56 | 1.3 | −1 |
| 07830 | S258P | | | | | |
| CMT24- | P009T-T078I-A202V | 0.7 | 0.5 | 35 | 0.9 | 0 |
| 07846 | | | | | | |
| CMT24- | V045R-G127T | 0.5 | 0.0 | 65 | 1.3 | 1 |
| 07934 | | | | | | |
| CMT24- | T077N-T078I-G165Q-G203E | 1.0 | 1.0 | 62 | 0.9 | −1 |
| 07870 | | | | | | |
| CMT24- | V045R-T078I-A202V- | 1.1 | 0.9 | 37 | 1 | 0 |
| 07910 | G203E | | | | | |
| CMT24- | G127T-G165Q-A202V- | 0.9 | 0.7 | 51 | 1.3 | 0 |
| 07687 | G203N | | | | | |
| CMT24- | A202V-K264H | 0.9 | 0.6 | 37 | 1 | −1 |
| 07695 | | | | | | |
| CMT24- | P009T-A202V-G203E | 1.0 | 0.9 | 41 | 1.2 | −1 |
| 07799 | | | | | | |
| CMT24- | T077N-A202V-G203E | 1.1 | 0.9 | 39 | 1.1 | −1 |
| 07943 | | | | | | |
| CMT24- | A202V-G203E-N217S | 1.0 | 0.8 | 36 | 1 | −1 |
| 07959 | | | | | | |
| CMT24- | Y103I-A202V | 0.7 | 0.2 | 40 | 1.1 | 0 |
| 07776 | | | | | | |
| CMT24- | T078I-G203N | 0.8 | 0.2 | 42 | 1 | 0 |
| 07704 | | | | | | |
| CMT24- | P009T-Q017H-T078I-G165Q | 0.9 | 0.5 | 51 | 1 | 0 |
| 07728 | | | | | | |
| CMT24- | Q017H-T078I | 0.6 | 0.1 | 45 | 1 | 0 |
| 07744 | | | | | | |
| CMT24- | S086D-A202V-G203E | 1.0 | 0.9 | 40 | 1.1 | −2 |
| 07944 | | | | | | |
| CMT24- | A202V-G203E-F260W | 0.9 | 0.8 | 36 | 0.9 | −1 |
| 07872 | | | | | | |
| CMT24- | G165Q-A202V-G203E- | 1.0 | 1.1 | 43 | 0.9 | −1 |
| 07960 | N217S | | | | | |
| CMT24- | G127T-G165Q-A202V- | 1.1 | 0.9 | 62 | 1.2 | −1 |
| 07888 | G203E | | | | | |
| CMT24- | P009T-Q017H-T078I-G127T | 0.8 | 0.3 | 37 | 1.4 | 0 |
| 07714 | | | | | | |
| CMT24- | V045R-S155E | 0.5 | 0.0 | 45 | 1.3 | 0 |
| 07795 | | | | | | |
| CMT24- | P009T-Q017H-T078I-A202V | 0.9 | 0.6 | 34 | 1.1 | 0 |
| 07723 | | | | | | |
| CMT24- | P009T-T078I | 0.5 | 0.1 | 45 | 1.1 | 0 |
| 07803 | | | | | | |
| CMT24- | S086D-S155E-A202V- | 1.0 | 1.0 | 41 | 1.1 | −3 |
| 07739 | G203E | | | | | |
| CMT24- | P009T-Q017H-A202V- | 1.1 | 0.9 | 42 | 1 | −1 |
| 07755 | G203E | | | | | |
| CMT24- | A024Q-G165Q-A202V- | 1.3 | 1.0 | 58 | 0.8 | −1 |
| 07859 | G203E | | | | | |
| CMT24- | P009T-Q017H-T077N- | 0.8 | 0.4 | 36 | 1.1 | 0 |
| 07939 | A202V | | | | | |
| CMT24- | Q017H-A202V-G203E | 1.0 | 0.9 | 36 | 1.1 | −1 |
| 07955 | | | | | | |
| CMT24- | P009T-G127T-A202V- | 1.0 | 0.7 | 48 | 1.3 | −1 |
| 07963 | G203E | | | | | |
| CMT24- | Y103I-G203E | 0.8 | 0.2 | 55 | 1.2 | −1 |
| 07987 | | | | | | |
| CMT24- | T078I-S155E-A202V-G203E | 1.1 | 1.0 | 35 | 1.1 | −2 |
| 08117 | | | | | | |
| CMT24- | N096D-S100E-Y103I | 0.4 | 0.0 | 45 | 1.3 | −2 |
| 08125 | | | | | | |
| CMT24- | Q017H-T078I-G127T- | 1.1 | 0.8 | 52 | 1.3 | 0 |
| 08133 | A202V | | | | | |
| CMT24- | P009T-N096D | 0.6 | 0.0 | 44 | 1.1 | −1 |
| 08054 | | | | | | |
| CMT24- | A128R-S155E | 0.8 | 0.1 | 54 | 1 | 0 |
| 08142 | | | | | | |
| CMT24- | G127T-A128R | 0.7 | 0.1 | 63 | 1.1 | 1 |
| 08111 | | | | | | |
| CMT24- | T078I-A202V | 0.7 | 0.4 | 24 | 0.9 | 0 |
| 08039 | | | | | | |
| CMT24- | P009T-A202V | 0.9 | 0.3 | 40 | 1.2 | 0 |
| 08135 | | | | | | |
| CMT24- | Q017H-A202V | 0.8 | 0.3 | 37 | 1 | 0 |
| 08056 | | | | | | |
| CMT24- | P009T-G203E | 0.8 | 0.4 | 39 | 1.1 | −1 |
| 08009 | | | | | | |
| CMT24- | T078I-G165Q-A202V | 1.1 | 0.9 | 45 | 1 | 0 |
| 08025 | | | | | | |
| CMT24- | T078I-A202V-G203E-N217S | 1.2 | 1.3 | 35 | 1 | −1 |
| 08121 | | | | | | |

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

What is claimed is:

1. A subtilisin variant comprising at least two, three or more amino acid substitutions selected from the group consisting of X9T, X17H, X45R, X68S, X78I, X86E, X87A, X96D, X100E, X100N, X103F, X103I, X108Q, X115L, X117R, X127S, X127T, X128K, X128P, X128R, X129Q, X155E, X161Q, X181E, X181Q, X202V, X203E, X203N, X217S, X221Q, X260W, and X264H, wherein the positions are numbered according to SEQ ID NO: 1, and wherein the variant has at least 75% identity to the amino acid sequence of SEQ ID NO: 1 or SEQ ID NO: 8.

2. The subtilisin variant of claim 1, wherein the variant has a) at least 25% improved stability in detergent as compared to the parent subtilisin SEQ ID NO: 1; and or b) a net charge of −4 to +2 at pH 8 relative to the subtilisin having the amino acid sequence of SEQ ID NO: 1.

3. The subtilisin variant of claim 1, wherein the variant further comprises one or more additional mutations selected from the group consisting of X24Q, X77N, X86D, X165Q, X184Q, X258D, and X258P, wherein the positions are numbered according to SEQ ID NO: 1.

4. The subtilisin variant of claim 1, wherein the variant comprises a set of substitutions selected from the group consisting of X9T-X17H, X9T-X45R, X9T-X68S, X9T-X78I, X9T-X86E, X9T-X87A, X9T-X96D, X9T-X100E, X9T-X100N, X9T-X103F, X9T-X103I, X9T-X108Q, X9T-X115L, X9T-X117R, X9T-X127S, X9T-X127T, X9T-X128K, X9T-X128P, X9T-X128R, X9T-X129Q, X9T-X155E, X9T-X161Q, X9T-X181E, X9T-X181Q, X9T-X202V, X9T-X203E, X9T-X203N, X9T-X217S, X9T-X260W, X9T-X221Q, X9T-X264H, X17H-X45R, X17H-X68S, X17H-X78I, X17H-X86E, X17H-X87A, X17H-X96D, X17H-X100E, X17H-X100N, X17H-X103F, X17H-X103I, X17H-X108Q, X17H-X115L, X17H-X117R, X17H-X127S, X17H-X127T, X17H-X128K, X17H-X128P, X17H-X128R, X17H-X129Q, X17H-X155E, X17H-X161Q, X17H-X181E, X17H-X181Q, X17H-X202V, X17H-X203E, X17H-X203N, X17H-X217S, X17H-X260W, X17H-X221Q, X17H-X264H, X45R-X68S, X45R-X78I, X45R-X86E, X45R-X87A, X45R-X96D, X45R-X100E, X45R-X100N, X45R-X103F, X45R-X103I, X45R-X108Q, X45R-X115L, X45R-X117R, X45R-X127S, X45R-X127T, X45R-X128K, X45R-X128P, X45R-X128R, X45R-X129Q, X45R-X155E, X45R-X161Q, X45R-X181E, X45R-X181Q, X45R-X202V, X45R-X203E, X45R-X203N, X45R-X217S, X45R-X260W, X45R-X221Q, X45R-X264H, X68S-X78I, X68S-X86E, X68S-X87A, X68S-X96D, X68S-X100E, X68S-X100N, X68S-X103F, X68S-X103I, X68S-X108Q, X68S-X115L, X68S-X117R, X68S-X127S, X68S-X127T, X68S-X128K, X68S-X128P, X68S-X128R, X68S-X129Q, X68S-X155E, X68S-X161Q, X68S-X181E, X68S-X181Q, X68S-X202V, X68S-X203E, X68S-X203N, X68S-X217S, X68S-X260W, X68S-X221Q, X68S-X264H, X78I-X86E, X78I-X87A, X78I-X96D, X78I-X100E, X78I-X100N, X78I-X103F, X78I-X103I, X78I-X108Q, X78I-X115L, X78I-X117R, X78I-X127S, X78I-X127T, X78I-X128K, X78I-X128P, X78I-X128R, X78I-X129Q, X78I-X155E, X78I-X161Q, X78I-X181E, X78I-X181Q, X78I-X202V, X78I-X203E, X78I-X203N, X78I-X217S, X78I-X260W, X78I-X221Q, X78I-X264H, X86E-X87A, X86E-X96D, X86E-X100E, X86E-X100N, X86E-X103F, X86E-X103I, X86E-X108Q, X86E-X115L, X86E-X117R, X86E-X127S, X86E-X127T, X86E-X128K, X86E-X128P, X86E-X128R, X86E-X129Q, X86E-X155E, X86E-X161Q, X86E-X181E, X86E-X181Q, X86E-X202V, X86E-X203E, X86E-X203N, X86E-X217S, X86E-X260W, X86E-X221Q, X86E-X264H, X87A-X96D, X87A-X100E, X87A-X100N, X87A-X103F, X87A-X103I, X87A-X108Q, X87A-X115L, X87A-X117R, X87A-X127S, X87A-X127T, X87A-X128K, X87A-X128P, X87A-X128R, X87A-X129Q, X87A-X155E, X87A-X161Q, X87A-X181E, X87A-X181Q, X87A-X202V, X87A-X203E, X87A-X203N, X87A-X217S, X87A-X260W, X87A-X221Q, X87A-X264H, X96D-X100E, X96D-X100N, X96D-X103F, X96D-X103I, X96D-X108Q, X96D-X115L, X96D-X117R, X96D-X127S, X96D-X127T, X96D-X128K, X96D-X128P, X96D-X128R, X96D-X129Q, X96D-X155E, X96D-X161Q, X96D-X181E, X96D-X181Q, X96D-X202V, X96D-X203E, X96D-X203N, X96D-X217S, X96D-X260W, X96D-X221Q, X96D-X264H, X100E-X100N, X100E-X103F, X100E-X103I, X100E-X108Q, X100E-X115L, X100E-X117R, X100E-X127S, X100E-X127T, X100E-X128K, X100E-X128P, X100E-X128R, X100E-X129Q, X100E-X155E, X100E-X161Q, X100E-X181E, X100E-X181Q, X100E-X202V, X100E-X203E, X100E-X203N, X100E-X217S, X100E-X260W, X100E-X221Q, X100E-X264H, X100N-X103F, X100N-X103I, X100N-X108Q, X100N-X115L, X100N-X117R, X100N-X127S, X100N-X127T, X100N-X128K, X100N-X128P, X100N-X128R, X100N-X129Q, X100N-X155E, X100N-X161Q, X100N-X181E, X100N-X181Q, X100N-X202V, X100N-X203E, X100N-X203N, X100N-X217S, X100N-X260W, X100N-X221Q, X100N-X264H, X103F-X103I, X103F-X108Q, X103F-X115L, X103F-X117R, X103F-X127S, X103F-X127T, X103F-X128K, X103F-X128P, X103F-X128R, X103F-X129Q, X103F-X155E, X103F-X161Q, X103F-X181E, X103F-X181Q, X103F-X202V, X103F-X203E, X103F-X203N, X103F-X217S, X103F-X260W, X103F-X221Q, X103F-X264H, X103I-X108Q, X103I-X115L, X103I-X117R, X103I-X127S, X103I-X127T, X103I-X128K, X103I-X128P, X103I-X128R, X103I-X129Q, X103I-X155E, X103I-X161Q, X103I-X181E, X103I-X181Q, X103I-X202V, X103I-X203E, X103I-X203N, X103I-X217S, X103I-X260W, X103I-X221Q, X103I-X264H, X108Q-X115L, X108Q-X117R, X108Q-X127S, X108Q-X127T, X108Q-X128K, X108Q-X128P, X108Q-X128R, X108Q-X129Q, X108Q-X155E, X108Q-X161Q, X108Q-X181E, X108Q-X181Q, X108Q-X202V, X108Q-X203E, X108Q-X203N, X108Q-X217S, X108Q-X260W, X108Q-X221Q, X108Q-X264H, X115L-X117R, X115L-X127S, X115L-X127T, X115L-X128K, X115L-X128P, X115L-X128R, X115L-X129Q, X115L-X155E, X115L-X161Q, X115L-X181E, X115L-X181Q, X115L-X202V, X115L-X203E, X115L-X203N, X115L-X217S, X115L-X260W, X115L-X221Q, X115L-X264H, X117R-X127S, X117R-X127T, X117R-X128K, X117R-X128P, X117R-X128R, X117R-X129Q, X117R-X155E, X117R-X161Q, X117R-X181E, X117R-X181Q, X117R-X202V, X117R-X203E, X117R-X203N, X117R-X217S, X117R-X260W, X117R-X221Q, X117R-X264H, X127S-X127T, X127S-X128K, X127S-X128P, X127S-X128R, X127S-X129Q, X127S-X155E, X127S-X161Q, X127S-X181E, X127S-X181Q, X127S-X202V, X127S-X203E, X127S-X203N, X127S-X217S, X127S-X260W, X127S-X221Q, X127S-X264H, X127T-X128K, X127T-X128P, X127T-X128R, X127T-X129Q, X127T-X155E, X127T-X161Q, X127T-X181E, X127T-X181Q, X127T-X202V, X127T-X203E, X127T-X203N, X127T-X217S, X127T-X260W, X127T-X221Q, X127T-X264H, X128K-X128P, X128K-X128R, X128K-X129Q, X128K-X155E, X128K-X161Q, X128K-X181E, X128K-X181Q, X128K-X202V, X128K-X203E, X128K-X203N, X128K-X217S, X128K-X260W, X128K-X221Q, X128K-X264H, X128P-X128R, X128P-X129Q, X128P-X155E, X128P-X161Q, X128P-X181E, X128P-X181Q, X128P-X202V, X128P-X203E, X128P-X203N, X128P-X217S, X128P-X260W, X128P-X221Q, X128P-X264H, X128R-X129Q, X128R-X155E, X128R-X161Q, X128R-X181E, X128R-X181Q, X128R-X202V, X128R-X203E, X128R-X203N, X128R-X217S, X128R-X260W, X128R-X221Q, X128R-X264H, X129Q-X155E, X129Q-X161Q, X129Q-X181E, X129Q-X181Q, X129Q-X202V, X129Q-X203E, X129Q-X203N, X129Q-X217S, X129Q-X260W, X129Q-X221Q, X129Q-X264H, X155E-X161Q, X155E-X181E, X155E-X181Q, X155E-X202V, X155E-X203E, X155E-X203N, X155E-X217S, X155E-X260W, X155E-X221Q, X155E-X264H, X161Q-X181E, X161Q-X181Q, X161Q-X202V, X161Q-X203E, X161Q-X203N, X161Q-X217S, X161Q-X260W, X161Q-X221Q, X161Q-X264H, X181E-X181Q, X181E-X202V, X181E-X203E, X181E-X203N, X181E-X217S, X181E-X260W, X181E-X221Q, X181E-X264H, X181Q-X202V, X181Q-X203E, X181Q-X203N, X181Q-X217S, X181Q-X260W, X181Q-X221Q, X181Q-X264H, X202V-X203E, X202V-X203N, X202V-X217S, X202V-X260W, X202V-X221Q, X202V-X264H, X203E-X203N, X203E-X217S, X203E-X260W, X203E-X221Q, X203E-X264H, X203N-X217S, X203N-X260W, X203N-X221Q, X203N-X264H, X217S-X260W, X217S-X221Q, X217S-X264H, X260W-X221Q, X260W-X264H, and X221Q-X264H, wherein the positions are numbered according to SEQ ID NO: 1.

5. The subtilisin variant of claim 1, wherein the variant comprises a set of substitutions selected from the group consisting of Q017H-N096D-G127T; Q017H-N096D-Y103F-A202V; G127T-A128K-S129Q-N184Q; P009T-Y103F-A202V-G203E; N096D-S100E-G127T-A202V; V087A-S155E-G165Q; P009T-T078I-Y103F-G127T; V087A-A202V-G203E; V045R-T161Q-S181Q-G203E; S155E-M221Q; T078I-Y103F; N096D-S100E-G127T-N217S; G127T-A128K-S129Q; T115L-G127T; P009T-T078I-N096D-A202V; V045R-S086E-S155E-A202V; V045R-T078I-Y103F-A202V; P009T-A202V-G203E-M221Q; A202V-G203E-M221Q-K264H; N096D-G127T; P009T-Q017H-Y103F-A202V; V045R-G127T-A128P-S129Q; S100E-G127T-A202V; A202V-M221Q; S100N-Y103I-G127T; G127T-A128K-S129Q-N217S; G127S-A128K-S129Q-A202V; T078I-N096D; A128K-S155E; N096D-Y103F-G165Q; S086E-T077N-T078I; V045R-T078I-S086E-A202V; N096D-S100E-G127S; T078I-G127T-A128K-S129Q; G127S-A128K-S129Q; Q017H-S086E-A202V-G203E; T161Q-S181Q-A202V-G203E; V045R-N096D-S100E; V045R-Y103F-G127T-A202V; N096D-S100E-G117R-G127T; V087A-G165Q-A202V; G127T-A128P-S129Q; V087A-M221Q; T115L-A202V-G203E; V045R-T078I-N096D-A202V; G127T-A128R-S129Q-G165Q; S100E-Y103I; S086E-S155E-A202V-G203E; N096D-Y103I-G127T; Q017H-N096D-Y103F; V045R-T078I-N096D-Y103F; S108Q-G127T; T078I-S086E-A202V-G203E; T078I-Y103F-G127T-A202V; S181E-A202V-G203E-S258D; N096D-A202V-G203E; N096D-Y103I-G127T-M221Q; V087A-G165Q-A202V-G203E; P009T-G127T-A128K-S129Q; S100E-G127T; S100N-M221Q; G127T-A128K-S129Q-A202V; V087A-S100N; S100E-G117R-G127T; T077N-G127T-A128K-S129Q; Q017H-N096D-G127T-A202V; N096D-A202V-M221Q; V045R-N096D-Y103F-G127T; N096D-G127T-G203E; V045R-S086E-A202V-G203E; T078I-S086E-S155E-G203E; V045R-M221Q; Q017H-N096D-Y103F-G127T; Q017H-Y103F-G127T-A202V; T078I-A202V-M221Q; V045R-N096D-G127T; V045R-N096D-G127T-A202V; P009T-T078I-Y103F-A202V; S100N-T115L; S086E-A202V; N096D-Y103F-G127T; T078I-N096D-G127T; T115L-G165Q-A202V; T078I-S086E; V045R-T078I-N096D-G127T; P009T-N096D-G127T; G127T-A128R-S129Q-A202V; T078I-N096D-A202V; N096D-Y103I-G203E; G127S-A128P-S129Q-A202V; N096D-G127T-N217S; V045R-T078I; A024Q-T078I-A202V; A202V-G203N; P009T-T077N-T078I; G203E-K264H; T077N-T078I-G165Q-A202V; V045R-T077N-G165Q-A202V; G165Q-A202V-G203E-S258P; P009T-T078I-A202V; V045R-G127T; T077N-T078I-G165Q-G203E; V045R-T078I-A202V-G203E; G127T-G165Q-A202V-G203N; A202V-K264H; P009T-A202V-G203E; T077N-A202V-G203E; A202V-G203E-N217S; Y103I-A202V; T078I-G203N; P009T-Q017H-T078I-G165Q; Q017H-T078I; S086D-A202V-G203E; A202V-G203E-F260W; G165Q-A202V-G203E-N217S; G127T-G165Q-A202V-G203E; P009T-Q017H-T078I-G127T; V045R-S155E; P009T-Q017H-T078I-A202V; P009T-T078I; S086D-S155E-A202V-G203E; P009T-Q017H-A202V-G203E; A024Q-G165Q-A202V-G203E; P009T-Q017H-T077N-A202V; Q017H-A202V-G203E; P009T-G127T-A202V-G203E; Y103I-G203E; T078I-S155E-A202V-G203E; N096D-S100E-Y103I; Q017H-T078I-G127T-A202V; P009T-N096D; A128R-S155E; G127T-A128R; T078I-A202V; P009T-A202V; Q017H-A202V; P009T-G203E; T078I-G165Q-A202V; T078I-A202V-G203E-N217S; and T077N-T078I-M221Q; wherein the positions are numbered according to SEQ ID NO: 1.

6. The subtilisin variant of claim 1, wherein said variant comprises an amino acid sequence with 80%, 85%, 86%, 87%, 88%, 89%, 90%, 91%, 92%, 93%, 94%, 95%, 96%, 97%, 98%, 99% or less than 100% amino acid sequence identity to SEQ ID NO: 1.

7. The subtilisin variant of claim 1, wherein said variant has one or more improved property when compared to a parent or reference subtilisin; wherein the improved property is selected from improved cleaning performance in detergent, improved stability in detergent; improved aged cleaning performance, and combinations thereof.

8. The subtilisin variant of claim 7, wherein the improved property is
(i) an improved cleaning performance in detergent, wherein said variant has a blood aged on cotton (CS-01) cleaning PI≥1.1 compared to the subtilisin having the amino acid sequence of SEQ ID NO: 8; and/or,
(ii) an improved cleaning performance in detergent, wherein said variant has a chocolate rice pudding aged on cotton (CS-100) cleaning PI≥1.1 compared to the subtilisin having the amino acid sequence of SEQ ID NO: 8; and/or
(iii) an improved cleaning performance in detergent, wherein said variant has a full egg with carbon black aged on cotton (C-S-39) cleaning PI≥1.1 compared to the subtilisin having the amino acid sequence of SEQ ID NO: 8; and/or,
(iv) an improved cleaning performance in detergent, wherein said variant has a chocolate soymilk drink aged on cotton (C-S-45) cleaning PI≥1.1 compared to the subtilisin having the amino acid sequence of SEQ ID NO: 8; and/or,
(v) an improved cleaning performance in detergent, wherein said variant has a grass on cotton (CS-07) cleaning PI≥1.1 compared to the subtilisin having the amino acid sequence of SEQ ID NO: 8; and/or,
(vi) an improved cleaning performance in detergent, wherein said variant has a chocolate milk with carbon black on cotton (C-03) cleaning PI≥1.1 compared to the subtilisin having the amino acid sequence of SEQ ID NO: 8; and/or,
(vii) an improved cleaning performance in detergent, wherein said variant has a blood/milk/ink on woven cotton (CS-05) cleaning PI≥1.1 compared to the subtilisin having the amino acid sequence of SEQ ID NO: 8; and/or,
(viii) an improved cleaning performance in detergent, wherein said variant has a milk with carbon black on cotton (C-11) cleaning PI≥1.1 compared to the subtilisin having the amino acid sequence of SEQ ID NO: 8; and/or,
(ix) an improved cleaning performance in detergent, wherein said variant has a blood/milk/ink on polycotton (EMPA 116) cleaning PI≥1.1 compared to the subtilisin having the amino acid sequence of SEQ ID NO: 8; and/or,
(x) an improved cleaning performance in detergent, wherein said variant has a blood aged on polyester/cotton (KCS-01) cleaning PI≥1.1 compared to the subtilisin having the amino acid sequence of SEQ ID NO: 8; and/or,
(xi) an improved stability, wherein said variant has a higher residual activity compared to the subtilisin having the amino acid sequence of SEQ ID NO: 1 or SEQ ID NO: 8 when measured in accordance with the stability assay of Example 2; and/or,
(xii) an improved aged cleaning performance measured as the difference in stain removal measured for a sample of aged test sample (where the enzyme is pre-incubated in detergent for an extended period of time such as 3-8 weeks at an elevated temperature such as 37° C.) compared to the aged cleaning performance of SEQ ID NO: 8.

9. The subtilisin variant of claim 7, wherein the improved property is
(i) proteolytic activity on DMC assay; and/or,
(ii) an improved stability in detergent, wherein said variant has a higher residual activity compared to the subtilisin having the amino acid sequence of SEQ ID NO: 1; and/or,
(iii) an improved cleaning performance in detergent, wherein said variant has a blood/milk/ink on polycotton (EMPA 116) cleaning PI≥1.1 compared to the subtilisin having the amino acid sequence of SEQ ID NO: 1
(iv) a net charge at pH 8 between-4 and +2,
and wherein the subtilisin variant has at least 75% sequence identity to SEQ ID NO:1.

10. An enzyme composition comprising one or more subtilisin variant according to claim 1.

11. The enzyme composition according to claim 10, further comprising one or more other enzymes selected from acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, dispersins, endo-beta-1,4-glucanases, endo-beta-mannanases, esterases, exo-mannanases, galactanases, glucoamylases, hemicellulases, hexosaminidase, hyaluronidases, keratinases, laccases, lactases, ligninases, lipases, lipoxygenases, lysozymes, mannanases, metalloproteases, nucleases (e.g. DNases and/or RNases), oxidases, oxidoreductases, pectate lyases, pectin acetyl esterases, pectinases, pentosanases, perhydrolases, peroxidases, phenoloxidases, phosphatases, phospholipases, phosphodiesterases, phytases, polygalacturonases, polyesterases, proteases, pullulanases, reductases, rhamnogalacturonases, beta-glucanases, tannases, transglutaminases, xanthan lyases, xylan acetyl-esterases, xylanases, xyloglucanases, xylosidases, and any combination or mixture thereof.

12. A polynucleotide comprising a nucleic acid sequence encoding a variant of claim 1 wherein said polynucleotide is, optionally, isolated.

13. The polynucleotide of claim 12, wherein the nucleic acid sequence is operably linked to a promoter.

14. An expression vector or cassette comprising the polynucleotide of claim 12.

15. A recombinant host cell comprising the polynucleotide of claim 12.

16. A cleaning composition comprising the subtilisin variant of claim 1 and at least one adjunct.

17. The cleaning composition of claim 16, wherein the cleaning composition is a composition selected from the group consisting of a laundry detergent, a fabric softening detergent, a dishwashing detergent (e.g., automatic or hand dishwashing detergents), a hard-surface cleaning detergent, and a medical instrument cleaning composition.

18. The cleaning composition of claim 17, wherein the detergent composition does not comprise a synthetic or peptidic protease stabilizer.

19. A method of cleaning comprising, contacting a surface or an item in need of cleaning with an effective amount of a subtilisin variant of claim 1 or the enzyme composition of claim 10; and optionally further comprising the step of rinsing said surface or item after contacting said surface or item with said variant or enzyme composition.

20. The method of claim 19, wherein said item is dishware, or fabric, or a medical instrument.
